(19) United States
(12) Patent Application Publication
(10) Pub. No.: US 2020/0085789 A1
(43) Pub. Date: Mar. 19, 2020

(54) USE OF A PROTEASOME INHIBITOR FOR THE TREATMENT OF CENTRAL NERVOUS SYSTEM (CNS) CANCERS

(71) Applicant: Celgene International II Sàrl, Couvet (CH)

(72) Inventors: Mohit TRIKHA, La Jolla, CA (US); Nancy LEVIN, La Jolla, CA (US); Benjamin WINOGRAD, West Orange, NJ (US)

(21) Appl. No.: 16/493,155

(22) PCT Filed: Mar. 7, 2018
(86) PCT No.: PCT/US18/21293

§ 371 Date: Sep. 11, 2019

Related U.S. Application Data
(60) Provisional application No. 62/471,318, filed on Mar. 14, 2017.
(60) Provisional application No. 62/491,939, filed on Apr. 28, 2017.
(60) Provisional application No. 62/517,653, filed on Jun. 9, 2017.
(60) Provisional application No. 62/586,412, filed on Nov. 15, 2017.
(60) Provisional application No. 62/615,185, filed on Jan. 9, 2018.
(60) Provisional application No. 62/622,324, filed on Jan. 26, 2018.

(51) Int. Cl. A61K 31/407; A61K 31/4015; A61N 5/10; A61P 25/00; A61P 35/00; C07K 16/22; A61N 1/36

(52) U.S. Cl. CPC A61K 31/407; A61K 31/4015; A61N 5/10; A61K 45/06; A61P 35/00; C07K 16/22; A61N 1/36002; A61P 25/00

(57) ABSTRACT

The present disclosure is related to dosage strategies for the treatment of CNS cancers with proteasome inhibitors (e.g., marizomib). For instance, the disclosure is related to strategies in which a proteasome inhibitor (e.g., marizomib) is administered at the same or higher dosage even after a subject has experienced a CNS-related adverse event.

### CROSS-REFERENCE TO RELATED APPLICATIONS

This application claims priority to, and benefit of, U.S. Provisional Application No. 62/471,318, filed Mar. 14, 2017; U.S. Provisional Application No. 62/491,939, filed Apr. 28, 2017; U.S. Provisional Application No. 62/517,653, filed Jun. 9, 2017; U.S. Provisional Application No. 62/586,412, filed Nov. 15, 2017; U.S. Provisional Application No. 62/615,185, filed Jan. 9, 2018; and United States Provisional Application No. 62/622,324, filed Jan. 26, 2018. The contents of each of these applications are incorporated by reference in their entirety.

### FIELD OF THE INVENTION

The present disclosure is related to dosing strategies for the treatment of CNS cancer using proteasome inhibitor (e.g., marizomib), wherein the administration of the proteasome inhibitor persists after the subject experiences a CNS adverse event.

### BACKGROUND OF THE INVENTION

Marizomib is an irreversible proteasome inhibitor.

Gliomas account for about 80% of primary malignant tumors in the central nervous system (CNS), with WHO Grade IV malignant glioma (G4 MG; including glioblastoma and gliosarcoma) constituting the majority of gliomas, and are essentially incurable. Currently only surgical resection and radiotherapy (RT) with concomitant and adjuvant temozolomide (TMZ) are standard-of-care treatment strategies for newly diagnosed G4 MG. However, resistance to chemotherapy and radiotherapy results in a high recurrence rate, with median survival of ˜15-16 months. Since no survival advantage has been demonstrated for the addition of bevacizumab (BEV) to temozolomide and radiotherapy in newly diagnosed G4 MG, alternative promising investigational agents need to be tested.

There is an unmet need for proteasome inhibitors capable of crossing the blood-brain barrier for the treatment of CNS or brain cancers (e.g., malignant glioma, glioblastoma, or CNS-multiple myeloma primary CNS lymphoma). There is also a need for dosing strategies capable of providing an efficacious dose of for treatment of brain cancers.

### SUMMARY OF THE INVENTION

In one aspect, the present disclosure provides a method of treating a central nervous system cancer in a subject in need thereof, the method comprising a treatment regimen comprising administering to the subject a therapeutic amount of a proteasome inhibitor, wherein the therapeutic amount, in the context of the treatment regimen, is sufficient for the subject to experience at least one central nervous system-related adverse event and wherein administration of the therapeutic amount is continued once the adverse event is triggered.

In some embodiments, the central nervous system-related adverse event is triggered in the cerebellum, brain, or brain stem. In some embodiments, the proteasome inhibitor is capable of crossing the blood-brain barrier. In some embodiments, the proteasome inhibitor is marizomib. In some embodiments, the central nervous system cancer is glioma. In some embodiments, the glioma is recurrent glioma. In some embodiments, the glioma is grade IV malignant glioma. In some embodiments, the glioma is glioblastoma.

In some embodiments, the subject experiences at least one central nervous system-related adverse event selected from the group consisting of ataxia, gait disturbance, fall, dysarthria, dizziness, and hallucination. In some embodiments, the subject experiences at least two central nervous system-related adverse events selected from the group consisting of ataxia, gait disturbance, fall, dysarthria, dizziness, and hallucination. In some embodiments, the subject experiences at least three central nervous system-related adverse events selected from the group consisting of ataxia, gait disturbance, fall, dysarthria, dizziness, and hallucination. In some embodiments, the subject experiences at least four central nervous system-related adverse events selected from the group consisting of ataxia, gait disturbance, fall, dysarthria, dizziness, and hallucination. In some embodiments, the subject experiences at least five central nervous system-related adverse events selected from the group consisting of ataxia, gait disturbance, fall, dysarthria, dizziness, and hallucination. In some embodiments, the subject experiences all of ataxia, gait disturbance, fall, dysarthria, dizziness, and hallucination. In some embodiments, the dizziness is balance disorder. In some embodiments, the subject further experiences a central nervous system-related adverse event selected from the group consisting of agitation, anxiety, aphasia, apraxia, cognitive disorder, concentration impairment, confusional state, convulsion, delirium, delusion, depressed level of consciousness, depression, facial nerve disorder, facial paresis, fatigue, insomnia, intention tremor, irritability, memory impairment, mental status change, personality change, psychotic disorder, pyramidal tract syndrome, somnolence, suicidal ideation, tremor, trigeminal nerve disorder, vertigo, or a combination thereof.

In some embodiments, the subject is administered increasing amounts of the proteasome inhibitor until the subject experiences the central nervous system-related adverse event. In some embodiments, administration of the proteasome inhibitor to the subject is continued after the subject experiences the central nervous system-related adverse event. In some embodiments, the dose of the proteasome inhibitor is not lowered after the subject experiences the central nervous system-related adverse event. In some embodiments, the adverse event is at least a grade 1 adverse event. In some embodiments, the adverse event is at least a grade 2 adverse event. In some embodiments, the adverse event is at least a grade 3 adverse event. In some embodiments, the adverse event is at least a grade 4 adverse event. In some embodiments, the proteasome inhibitor is administered weekly. In some embodiments, the proteasome inhibitor is administered in combination with bevacizumab.

In another aspect, the present disclosure provides a method of determining therapeutic amount of a proteasome inhibitor for the treatment of a central nervous system cancer in a subject in need thereof, the method comprising a treatment regimen comprising administering to the subject the proteasome inhibitor at increasing dose amounts until the subject experiences at least one central nervous system-related adverse event, wherein the therapeutic amount, in the context of the treatment regimen, is the amount at which the subject experiences the central nervous system-related adverse event.

In some embodiments, the central nervous system-related adverse event is triggered in the cerebellum, brain, or brain stem. In some embodiments, the subject is administered a first subsequent dose of proteasome inhibitor if no adverse events are experienced by the subject after being administered an initial dose of proteasome inhibitor, wherein the first subsequent dose comprises a greater amount of proteasome inhibitor than the initial dose.

In some embodiments, the subject is administered a second subsequent dose of proteasome inhibitor if no central nervous system-related adverse events are experienced by the subject after being administered the first subsequent dose, wherein the second subsequent dose comprises a greater amount of proteasome inhibitor than the first subsequent dose. In some embodiments, the initial dose is about 0.55 mg/m² of proteasome inhibitor. In some embodiments, the first subsequent dose is about 0.7 mg/m² of proteasome inhibitor. In some embodiments, the second subsequent dose is about 0.8 mg/m² of proteasome inhibitor. In some embodiments, the initial dose is about 0.8 mg/m² of proteasome inhibitor. In some embodiments, the first subsequent dose is about 1.1 mg/m² of proteasome inhibitor. In some embodiments, the first subsequent dose is about 1.2 mg/m² of proteasome inhibitor. In some embodiments, the proteasome inhibitor is capable of crossing the blood-brain barrier.

In some embodiments, the proteasome inhibitor is marizomib. In some embodiments, the central nervous system cancer is glioma. In some embodiments, the glioma is recurrent glioma. In some embodiments, the glioma is grade IV malignant glioma. In some embodiments, the glioma is glioblastoma.

In some embodiments, the subject experiences at least one central nervous system-related adverse event selected from the group consisting of ataxia, gait disturbance, fall, dysarthria, and dizziness, and hallucination or a combination thereof. In some embodiments, the subject experiences at least two central nervous system-related adverse events selected from the group consisting of ataxia, gait disturbance, fall, dysarthria, and dizziness, and hallucination or a combination thereof. In some embodiments, the subject experiences at least three central nervous system-related adverse events selected from the group consisting of ataxia, gait disturbance, fall, dysarthria, and dizziness, and hallucination or a combination thereof. In some embodiments, the subject experiences at least four central nervous system-related adverse events selected from the group consisting of ataxia, gait disturbance, fall, dysarthria, and dizziness, and hallucination or a combination thereof. In some embodiments, the subject experiences at least five central nervous system-related adverse events selected from the group consisting of ataxia, gait disturbance, fall, dysarthria, and dizziness, and hallucination or a combination thereof. In some embodiments, the subject experiences ataxia, gait disturbance, fall, dysarthria, and dizziness, and hallucination. In some embodiments, the at least one adverse event furthers includes hallucination, agitation, anxiety, aphasia, apraxia, cognitive disorder, concentration impairment, confusional state, convulsion, delirium, delusion, depressed level of consciousness, depression, facial nerve disorder, facial paresis, fatigue, insomnia, intention tremor, irritability, memory impairment, mental status change, personality change, psychotic disorder, pyramidal tract syndrome, somnolence, suicidal ideation, tremor, trigeminal nerve disorder, vertigo, or a combination thereof.

In some embodiments, the adverse event is at least a grade 1 adverse event. In some embodiments, the adverse event is at least a grade 2 adverse event. In some embodiments, the adverse event is at least a grade 3 adverse event. In some embodiments, the adverse event is at least a grade 4 adverse event. In some embodiments, the proteasome inhibitor is administered weekly. In some embodiments, the therapeutic amount is sufficient to treat a cancer in subjects with a methylated MGMT promoter. In some embodiments, the therapeutic amount is sufficient to treat a cancer in subjects with an un-methylated MGMT promoter. In some embodiments, the therapeutic amount is sufficient to treat a cancer in subjects with a methylated MGMT promoter. In some embodiments, the therapeutic amount is sufficient to treat a cancer in subjects with an un-methylated MGMT promoter. In some embodiments, the treatment regimen is the proteasome inhibitor alone. In some embodiments, the treatment regimen comprises the proteasome inhibitor in combination with an additional therapeutic agent. In some embodiments, the additional therapeutic agent is bevacizumab. In some embodiments, the proteasome inhibitor is marizomib alone. In some embodiments, the treatment regimen is the proteasome inhibitor alone. In some embodiments, the treatment regimen comprises the proteasome inhibitor in combination with an additional therapeutic agent. In some embodiments, the additional therapeutic agent is bevacizumab. In some embodiments, the proteasome inhibitor is marizomib.

### BRIEF DESCRIPTION OF THE FIGURES

FIG. 1A shows progression free survival (PFS) according to whether patients suffered ataxia, gait disturbance, fall, dysarthria, dizziness, including balance disorders, and hallucinations.

FIG. 1B shows overall survival (OS) according to whether patients suffered ataxia, gait disturbance, fall, dysarthria, dizziness, including balance disorders, and hallucinations.

FIG. 1C shows progression free survival (PFS) according to whether patients suffered ataxia, gait disturbance, fall, dysarthria, and dizziness, including balance disorders.

FIG. 1D shows overall survival (OS) according to whether patients suffered ataxia, gait disturbance, fall, dysarthria, and dizziness, including balance disorders.

FIG. 2A shows progression free survival (PFS) according to whether patients suffered anxiety.

FIG. 2B shows overall survival (OS) according to whether patients suffered anxiety.

FIG. 3A shows progression free survival (PFS) according to whether patients suffered aphasia.

FIG. 3B shows overall survival (OS) according to whether patients suffered aphasia.

FIG. 4A shows progression free survival (PFS) according to whether patients suffered ataxia.

FIG. 4B shows overall survival (OS) according to whether patients suffered ataxia.

FIG. 5A shows progression free survival (PFS) according to whether patients suffered confusional state.

FIG. 5B shows overall survival (OS) according to whether patients suffered confusional state.

FIG. 6A shows progression free survival (PFS) according to whether patients suffered convulsions.

FIG. 6B shows overall survival (OS) according to whether patients suffered convulsions.

FIG. 7A shows progression free survival (PFS) according to whether patients suffered diarrhea.

FIG. 7B shows overall survival (OS) according to whether patients suffered diarrhea.

FIG. 8A shows progression free survival (PFS) according to whether patients suffered dizziness.

FIG. 8B shows overall survival (OS) according to whether patients suffered dizziness.

FIG. 9A shows progression free survival (PFS) according to whether patients suffered dysarthria.

FIG. 9B shows overall survival (OS) according to whether patients suffered dysarthria.

FIG. 10A shows progression free survival (PFS) according to whether patients suffered fall.

FIG. 10B shows overall survival (OS) according to whether patients suffered fall.

FIG. 11A shows progression free survival (PFS) according to whether patients suffered fatigue.

FIG. 11B shows overall survival (OS) according to whether patients suffered fatigue.

FIG. 12A shows progression free survival (PFS) according to whether patients suffered gait disturbance.

FIG. 12B shows overall survival (OS) according to whether patients suffered gait disturbance.

FIG. 13A shows progression free survival (PFS) according to whether patients suffered hallucinations.

FIG. 13B shows overall survival (OS) according to whether patients suffered hallucinations.

FIG. 14A shows progression free survival (PFS) according to whether patients suffered hypokalemia.

FIG. 14B shows overall survival (OS) according to whether patients suffered hypokalemia.

FIG. 15A shows progression free survival (PFS) according to whether patients suffered infusion site pain.

FIG. 15B shows overall survival (OS) according to whether patients suffered infusion site pain.

FIG. 16A shows progression free survival (PFS) according to whether patients suffered memory impairment.

FIG. 16B shows overall survival (OS) according to whether patients suffered memory impairment.

FIG. 17 shows a plot of the number of patients who did and did not experience hallucination as a function of time.

FIG. 18 shows a plot of the timing of hallucinations and dose reductions.

FIG. 19 shows the overall study design of a Phase 1 clinical trial set forth in Example 1.

FIG. 20 shows a plot of the response of patients by RANO gliomas set forth in Example 1.

FIG. 21 shows a plot of the time to progression for subjects as set forth in Example 1.

FIG. 22 shows nine MRI images of an example of target lesion complete response in Patient A gliomas set forth in Example 1.

FIG. 23 shows a plot of the tumor area as a function of time in Patient A set forth in Example 1.

FIG. 24 shows MRI images of Patient B as set forth in Example 1.

FIG. 25 shows a plot of Patient B's tumor size as a function of time and the number of cycles Patient B received as set forth in Example 1.

FIG. 26 shows MRI images of Patient C as set forth in Example 1.

FIG. 27 shows a plot of Patient C's tumor size as a function of time and the number of cycles Patient C received as set forth in Example 1.

FIG. 28 shows a plot of Patient D's tumor size as a function of time and the number of cycles Patient D received as set forth in Example 1.

FIG. 29 shows a plot of Patient E's tumor size as a function of time and the number of cycles Patient D received as set forth in Example 1.

FIG. 30A shows a plot of the PFS percent as a function of time in all patients treated with marizomib for glioma as set forth in Example 1.

FIG. 30B shows a plot of the OS percent as a function of time in all patients treated with marizomib for glioma as set forth in Example 1.

FIG. 31A shows a plot of the PFS percent by MGMT Promoter methylation status as a function of time after treatment with MRZ and BEV.

FIG. 31B shows a plot of the OS percent by MGMT Promoter methylation status as a function of time after treatment with MRZ and BEV.

FIG. 32A shows progression free survival (PFS) as a function of time for patients by EGFR status.

FIG. 32B shows overall survival (OS) as a function of time for patients by EGFR status.

FIG. 33 shows a time to progression for patients undergoing monotherapy with marizomib as set forth in Example 1.

FIG. 34A shows a plot of progression-free survival for patients treated with marizomib monotherapy by methylation status.

FIG. 34B shows a plot of overall survival for patients treated with marizomib monotherapy by methylation status.

FIG. 35 shows the concentration of marizomib in the blood of a patient C1D1 pre- and post-infusion.

FIG. 36 shows the concentration of bevacizumab in the serum of a patient C1D1 pre- and post-infusion.

FIG. 37 shows the concentration of marizomib in the blood as a function of time on C1D8.

FIG. 38 shows concentration of bevacizumab in serum pre- and post-infusion for different cohorts on C1D15.

FIG. 39 shows a time to progression for patients undergoing marizomib/bevacizumab (MRZ+BEV) dose-escalation as set forth in Example 4.

FIG. 40 shows a time to progression for 4 patients with prolonged disease stabilization undergoing marizomib (MRZ) monotherapy.

FIG. 41 shows a plot of overall survival (OS) for patients treated with marizomib (MRZ) monotherapy.

FIG. 42A shows progression free survival (PFS) according to whether patients suffered special interest adverse events (SIAEs) of ataxia, gait disturbance, fall, dysarthria, dizziness, including balance disorders, and hallucinations.

FIG. 42B shows overall survival (OS) according to whether patients suffered special interest adverse events (SIAEs) of ataxia, gait disturbance, fall, dysarthria, dizziness, including balance disorders, and hallucinations.

FIG. 43 shows a patient time on treatment for patients undergoing marizomib/bevacizumab (MRZ+BEV) intra-patient dose escalation as set forth in Example 4.

FIG. 44 shows a swimmer plot of concomitant cohort patients in dose-escalation as set forth in Example 5.

FIG. 45 shows a swimmer plot of adjuvant cohort patients in dose-escalation as set forth in Example 5.

### DETAILED DESCRIPTION OF THE INVENTION

Set forth herein is a strategy for determining an efficacious dose of a proteasome inhibitor (e.g., marizomib) by determining the dosage at which a subject being treated with a proteasome inhibitor experiences a CNS-adverse event.

Without wishing to be bound by theory, marizomib is the only known proteasome inhibitor capable of crossing the blood-brain barrier. Although other proteasome inhibitors are known to cause adverse events, marizomib is the only known proteasome inhibitor capable of producing CNS-adverse events (e.g., ataxia, gait disturbance, fall, dysarthria, and dizziness (including balance disorder) and hallucinations). For example, without wishing to be bound by theory, bortezomib, a proteasome inhibitor is known to cause adverse events in patients such as peripheral nephropathy and gastrointestinal events. However, bortezomib is not known to cause CNS adverse events. Similarly, without wishing to be bound by theory, carfilzomib is a proteasome inhibitor that is known to cause hematological adverse events. However, carfilzomib is not known to cause CNS adverse events. In contrast to bortezomib and carfilzomib, marizomib is known to produce CNS adverse events, however marizomib is not known to produce other adverse events such as peripheral nephropathy or hematological disorders. Without wishing to be bound by theory, this can be due to the fact that marizomib is capable of crossing the blood-brain-barrier and interacting with the brain. Accordingly, as set forth herein, an efficacious dose of marizomib for the treatment of brain cancer is determined when a subject experiences a CNS adverse event (e.g., ataxia, gait disturbance, fall, dysarthria, dizziness (including balance disorder) and/or hallucinations, for instance in the absence of other adverse events.

Without wishing to be bound by theory, a select subset of CNS adverse events are triggered in the cerebellum, where proteasome activity is highest and therefore likely to be most sensitive to marizomib. Proteasome activity in the cerebellum is thought to be higher than proteasome activity in both the cerebrum and the tumor, and proteasome activity in the cerebrum and tumor are thought to be roughly equal. Without wishing to be bound by theory, proteasome activity is thought to be lowest in the brainstem.

Without wishing to be bound by theory, proteasome activity in the cerebellum can be responsible for CNS adverse events such as ataxia, dizziness, dysarthria, fall, gait disturbance, hallucination, or a combination thereof. Without wishing to be bound by theory, proteasome activity in the cerebrum can be responsible for confusion, convulsions, memory impairment, or a combination thereof.

Without wishing to be bound by theory, in some cases medical practitioners will reduce the dosage of marizomib or end treatment with marizomib if a patient experiences an adverse event such as a CNS adverse event. However, as set forth herein, in some embodiments marizomib treatment is continued at the same and/or increased dosage as the dosage that led to a CNS-adverse event. In some embodiments, marizomib treatment is continued at the same and/or decreased dosage as the dosage that led to a CNS-adverse event, however the treatment is not stopped. Accordingly, set forth herein is a method for establishing an individualized treatment and dosage regimen for treatment of a patient suffering from a CNS cancer using marizomib. As set forth herein, a patient can be dosed until the patient experiences a CNS adverse event, and the patient continues to be treated with marizomib. It should be understood that the amount of the proteasome inhibitor (e.g. a therapeutic amount) is sufficient in the context of the overall treatment regimen being provided to produce a CNS adverse event. The overall treatment regimen can either be a proteasome inhibitor alone (e.g., marizomib) or a proteasome inhibitor in combination with an additional therapeutic agent (e.g., marizomib in combination with bevacizumab).

Without wishing to be bound by theory, it was found that patients who had at least one CNS adverse event lived longer (i.e., had greater overall survival, or OS) and had greater progression free survival (PFS) than patients who did not experience CNS-adverse events. In some embodiments, the CNS adverse event is selected from agitation, anxiety, aphasia, apraxia, cognitive disorder, concentration impairment, confusional state, convulsion, delirium, delusion, depressed level of consciousness, depression, facial nerve disorder, facial paresis, fatigue, insomnia, intention tremor, irritability, memory impairment, mental status change, personality change, psychotic disorder, pyramidal tract syndrome, somnolence, suicidal ideation, tremor, trigeminal nerve disorder, vertigo, or a combination thereof. In some embodiments, the CNS adverse event is selected from ataxia, gait disturbance, fall, dysarthria, and dizziness (including balance disorder) and hallucinations.

As set forth herein, a “patient” or a “subject” is a person who is suffering from a CNS cancer and is receiving treatment for that cancer (e.g., as set forth in Example 1).

As set forth herein, marizomib was evaluated in bevacizumab-naive grade-4 malignant glioma (G4 MG) patients. The patients had no prior anti-angiogenic or proteasome inhibition therapy, and had a Karnofsky score greater than 70. The Phase 1 (P1) marizomib plus bevacizumab (MRZ+BEV) study was a 3+3 MRZ dose-escalation study (0.55 mg/m² (6 patients), 0.7 mg/m² (3 patients), and 0.8 mg/m² (3 patients)) followed by a dose-expansion study (0.8 mg/m², 24 patients) in which safety and activity were assessed. The safety and activity of marizomib monotherapy (0.8 mg/m², 30 patients) were similarly assessed in the Phase 2 (P2) study. Treatments were administered intravenously in 28-day cycles: marizomib (10 min infusion) on days 1, 8, and 15; and bevacizumab infusion (10 mg/kg) on days 1 & 15. Tumor response was measured every other cycle by RANO criteria. Marizomib and bevacizumab pharmacokinetics and proteasome inhibition in circulating blood cells were evaluated in Phase 1 (P1).

In Phase 1, the mean age of patients was 55 years, 64% of patients were male, and the mean treatment duration was 5.3 cycles. In Phase 2, the mean age of patients was 56 years, 57% of patients were male, and the mean treatment duration was 2.5 cycles.

### Phase 1 Adverse Events

Study treatment-related Grade ≥3 Adverse Events (AEs) that occurred in two or more patients in Phase 1 were hypertension, headache, confusional state, fatigue, hallucination, proteinuria. Three grade 4 serious adverse events (SAEs) were reported: appendicitis perforated; depressed level of consciousness, (which was found not to be related); and blindness (which was found to be bevacizumab-related). Three Grade 5 SAEs were reported: 2 patients experienced disease progression, (which was found not to be related to treatment); and one patient experienced intracranial hemorrhage (which was found to be bevacizumab-related). One Phase 1 patient had a dose-limiting toxicity (DLT) (fatigue); no other dose-limiting toxicities occurred.

### Phase 2 Adverse Events

Study treatment-related Grade ≥3 adverse events (AEs) that occurred in two or more patients in Phase 2 were fatigue, hallucination, lethargy. One Grade 4 serious adverse event (hallucination, MRZ-related) was reported.

### Phase 1 Response

In Phase 1, the overall response rate was 44% (i.e., 16/36 patients experienced at least a partial response), including one complete response. Four patients experienced partial response with complete target lesion response; 11 patients experienced static disease, 6 patients experienced progressive disease, and 3 patients were not radiographically evaluable. Overall survival (OS) at 6/9/12 months was 75/60/39%, respectively; the median overall survival was 9.4 months. For patients with an unmethylated (e.g., less than about 8% methylation) MGMT promoter (n=22 patients), overall survival at 6/9/12 months was 68/45/15% respectively; the median overall survival was 7.2 mo. For patients with methylated (e.g., greater than about 8% methylation) MGMT promoter (n=10 patients), overall survival at 6/9/12 months was 78/78/67%, respectively. The median overall survival was not reached.

### Phase 2 Response

In Phase 2, the overall response rate was 3% (i.e., 1/30 patients experienced a partial response). Eight patients experienced static disease, 19 patients experienced progressive disease, and 2 patients were not evaluable.

### Phase 1 Response by Adverse Events

In Phase 1, for patients experiencing at least one of the most frequent CNS-related adverse events (any grade: ataxia, balance disorder, dizziness, dysarthria, fall, gait disturbance, hallucination), overall survival is increased (83/74/45% at 6/9/12 months, respectively, with a median overall survival of 11.4 months, n=23) compared with patients who did not experience at least one of the most frequent CNS-related adverse events (59/34/25% overall survival at 6/9/12 months, respectively, with a median overall survival of 6.3 months, n=13). Marizomib-related safety profiles were similar in Phase 1 and Phase 2. The marizomib and bevacizumab combination demonstrated substantial activity overall and in the unmethylated MGMT promoter subgroup compared with historic bevacizumab monotherapy publications. Without wishing to be bound by theory, recurrent grade 4 malignant glioma patients who experienced CNS adverse events demonstrated greater therapeutic benefit with marizomib and bevacizumab. In some embodiments, marizomib is dose-escalated in marizomib and bevacizumab treated patients who do not experience a CNS adverse event in the first dosing cycle at 0.8 mg/m².

Table 1A below shows a summary of the progression free survival (PFS) in patients who experienced one or more of ataxia, gait disturbance, fall, dysarthria, and dizziness (including balance disorder) and hallucinations (A/GD/F/D/D+H) compared with those who did not experience those symptoms (Non A/GD/F/D/D+H). Table 1B below shows a summary of the progression free survival (PFS) in patients who experienced one or more of ataxia, gait disturbance, fall, dysarthria, and dizziness (including balance disorder) (A/GD/F/D/D) compared with those who experienced none of those symptoms (Non A/GD/F/D/D). As set forth in Tables 1A and 1B below, patients who experienced any of one of ataxia, gait disturbance, fall, dysarthria, dizziness (including balance disorder) and hallucinations had longer progression free survival and overall survival than patients who did not experience those symptoms. The leftmost column in Tables 1A and 1B refer to whether the patient has experienced at least one of ataxia, gait disturbance, fall, dysarthria, dizziness (including balance disorder) and hallucinations. Given in columns 2-4 is the progression free survival (PFS) for patients, including the median PFS and the percentage of patients with PFS at 6 and 9 months. Given in columns 5-7 is the overall survival (OS) for patients, including the median PFS and the percentage of patients with OS at 6 and 9 months.

TABLE 1A
PFS and OS in Patients by Ataxia, Gait Disturbance, Fall, Dysarthria,
and Dizziness (including Balance Disorder) and Hallucinations

| | PFS (p = 0.01) | | | OS (p = 0.05) | | |
|---|---|---|---|---|---|---|
| | # | Median | 6/9 | # | Median | 6/9/12 |
| | Censored | (months) | mo % | Censored | (months) | mo % |
| A/GD/F/D/ | 5 | 5.5 | 46/31 | 11 | 10.4 | 82/72/29 |
| D + H (23) | | | | | | |
| Non | 3 | 2.8 | 9/9 | 5 | 6.3 | 58/39/Not |
| A/GD/F/D/ | | | | | | determined |
| D + H (13) | | | | | | |

TABLE 1B
PFS and OS in Patients by Ataxia, Gait Disturbance, Fall,
Dysarthria, and Dizziness (including Balance Disorder)

| | PFS | | | OS | | |
|---|---|---|---|---|---|---|
| | | | 9 | | | 6 |
| | # | Median | mo | # | Median | mo |
| | Censored | (months) | % | Censored | (months) | % |
| A/GD/F/D/ | 4 | 7.0 | 34 | 9 | 10.4 | 79 |
| D (20) | | | | | | |
| Non | 4 | 3.6 | 8 | 7 | 7.3 | 67 |
| A/GD/F/D/ | | | | | | |
| D (16) | | | | | | |

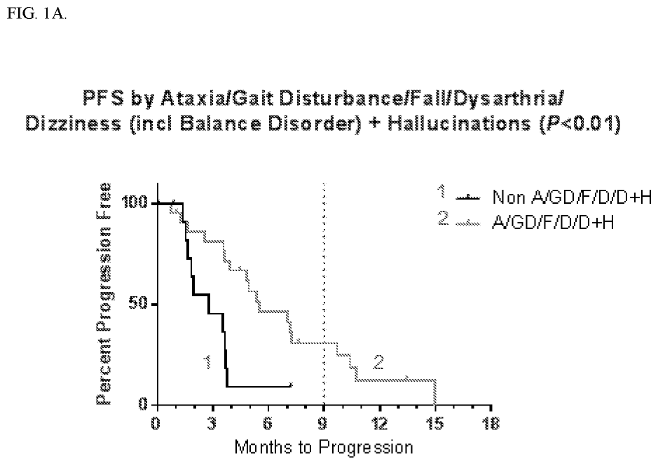

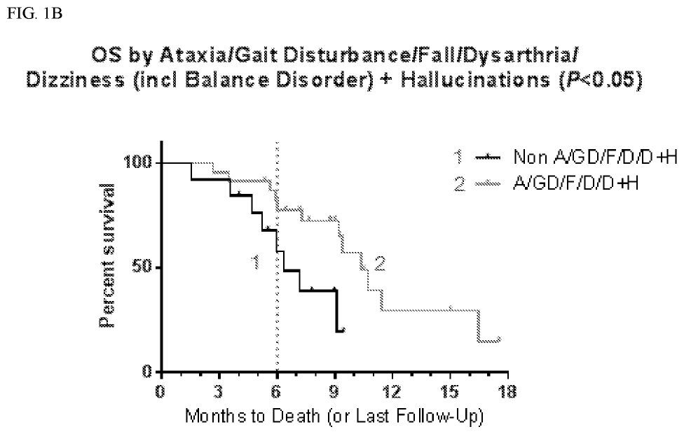

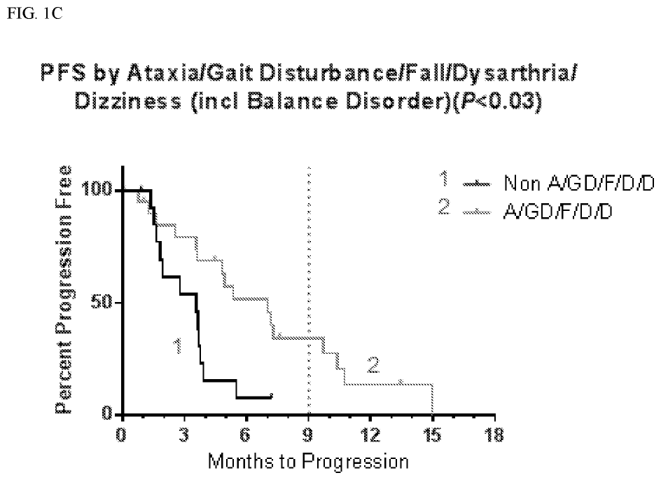

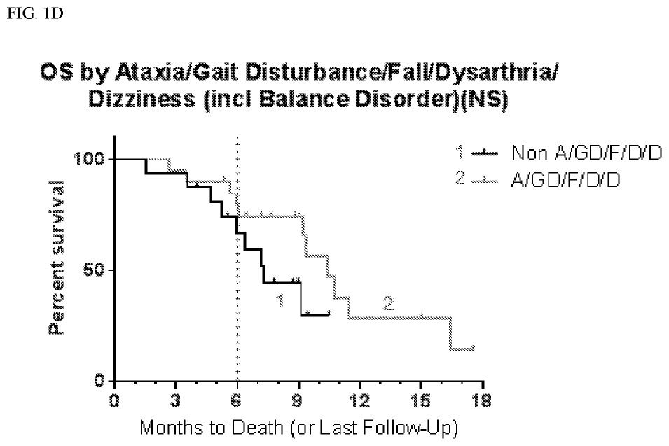

As set forth in Table 2 below, the response of patients to treatment with marizomib was independent of the methylation status of the patient's MGMT promoter. Without wishing to be bound by theory, a methylated MGMT promoter is understood to be a factor contributing to good prognosis for patients with CNS cancer. In contrast, unmethylated MGMT is understood as a factor contributing to bad prognosis for patients with CNS cancer. As set forth in Table 2, the response of patients who experienced a CNS-adverse event (e.g., ataxia, gait disturbance, fall, dysarthria, and dizziness (including balance disorder) and hallucinations) was similar regardless of whether or not the patients had methylated or unmethylated MGMT. The data from Table 2 was collected approximately six weeks after the data in Tables 1A and 1B and FIGS. 1-19.

TABLE 2
PFS and OS in Patients with CNS Adverse Events by MGMT Methylation Status

| | PFS | | | OS | | |
|---|---|---|---|---|---|---|
| | # | Median | 6/9 mo | # | Median | 6/9 mo |
| | Censored | (months) | % | Censored | (months) | % |
| Methylated | | | | | | |
| A/GD/F/D/D + | 2 | 5.49 | 34/17 | 6 | — | 88/88 |
| H (8) | | | | | | |
| Non | 1 | 1.94 | 0/0 | 1 | 5.23 | 0 |
| A/GD/F/D/D + | | | | | | |
| H (2) | | | | | | |
| Unmethylated | | | | | | |
| A/GD/F/D/D + | 1 | 7.01 | 53/35 | 2 | 9.21 | 77/62 |
| H (13) | | | | | | |
| Non | 0 | 2.76 | 0/0 | 1 | 5.99 | 38/19 |
| A/GD/F/D/D + | | | | | | |
| H (7) | | | | | | |
| Unknown | | | | | | |
| A/GD/F/D/D + | 0 | 10.16 | 50/50 | 2 | — | 100/100 |
| H (2) | | | | | | |
| Non | 2 | 3.68 | 33/33 | 3 | — | 100/75 |
| A/GD/F/D/D + | | | | | | |
| H (4) | | | | | | |

Set forth in Table 3 is an analysis of progression free survival (PFS) depending on whether or not subjects experienced CNS adverse events. As shown in Table 3, patients with CNS adverse events tended to have longer PFS.

TABLE 3
PFS by CNS Adverse Events

| | Affected | | | | Not Affected | | | |
|---|---|---|---|---|---|---|---|---|
| | # | # | mPFS | 9 mo | # | # | mPFS | 9 mo |
| AE | Total | Censored | (mo) | PFS (%) | Total | Censored | (mo) | PFS (%) |
| Anxiety | 6 | 0 | 5.5 | 33 | 30 | 8 | 3.9 | 21 |
| Aphasia | 6 | 0 | 3.2 | 17 | 30 | 8 | 4.8 | 24 |
| Ataxia | 8 | 2 | 4.9 | 31 | 28 | 6 | 3.7 | 20 |
| Confusion | 10 | 2 | 6.0 | 25 | 26 | 6 | 3.7 | 23 |
| Convulsion | 7 | 0 | 3.6 | 29 | 29 | 8 | 3.9 | 21 |
| Diarrhea | 10 | 2 | 5.4 | 48 | 26 | 6 | 3.8 | 12 |
| Dizziness | 10 | 1 | 5.1 | 10 | 26 | 7 | 3.7 | 31 |
| Dysarthria | 7 | 1 | 7.1 | 33 | 29 | 7 | 3.8 | 20 |
| Fall | 10 | 2 | 7.2 | 45 | 26 | 6 | 3.7 | 13 |
| Fatigue | 26 | 5 | 4.8 | 25 | 10 | 3 | 3.3 | 19 |
| Gait | 5 | 2 | 5.2 | 40 | 31 | 6 | 3.7 | 20 |
| disturbance | | | | | | | | |
| Hallucination | 13 | 2 | 5.4 | 24 | 23 | 6 | 3.7 | 19 |
| Hypokalemia | 7 | 1 | 7.1 | 38 | 29 | 7 | 3.8 | 19 |
| Inf. Site Pain | 8 | 0 | 5.5 | 38 | 28 | 8 | 3.9 | 18 |
| Memory | 7 | 2 | 10.4 | 57 | 29 | 6 | 3.7 | 13 |
| impairment | | | | | | | | |

Set forth in Table 4 is an analysis of overall survival (OS) depending on whether or not subjects experienced CNS adverse events. As shown in Table 4, patients with CNS adverse events tended to have longer OS.

TABLE 4
OS by CNS Adverse Events

| | Affected | | | | Not Affected | | | |
|---|---|---|---|---|---|---|---|---|
| | # | # | mOS | 6 mo OS | # | # | mOS | 6 mo OS |
| AE | Total | Censored | (mo) | (%) | Total | Censored | (mo) | (%) |
| Anxiety | 6 | 2 | 10.7 | 83 | 30 | 14 | 9.2 | 72 |
| Aphasia | 6 | 2 | 6.0 | 44 | 30 | 14 | 9.4 | 79 |
| Ataxia | 8 | 3 | 9.2 | 73 | 28 | 13 | 9.4 | 74 |
| Confusion | 10 | 5 | 10.4 | 68 | 26 | 11 | 9.4 | 76 |
| Convulsion | 7 | 3 | 10.4 | 54 | 29 | 13 | 9.4 | 79 |
| Diarrhea | 10 | 5 | 10.7 | 90 | 26 | 11 | 9.1 | 68 |
| Dizziness | 10 | 5 | 11.4 | 70 | 26 | 11 | 9.4 | 76 |
| Dysarthria | 7 | 1 | 9.4 | 86 | 29 | 15 | 11.4 | 71 |
| Fall | 10 | 4 | 9.4 | 80 | 26 | 12 | 9.1 | 71 |
| Fatigue | 26 | 11 | 9.4 | 72 | 10 | 5 | 10.3 | 80 |
| Gait | 5 | 4 | Not | 80 | 31 | 12 | 9.2 | 73 |
| disturbance | | | defined | | | | | |
| Hallucination | 13 | 8 | 10.4 | 92 | 23 | 8 | 9.1 | 64 |
| Hypokalemia | 7 | 3 | 9.2 | 86 | 29 | 13 | 9.4 | 72 |
| Inf Site Pain | 8 | 2 | 10.4 | 75 | 28 | 14 | 9.2 | 74 |
| Memory | 7 | 4 | 10.4 | 86 | 29 | 12 | 9.1 | 71 |
| impairment | | | | | | | | |

### Dosing

In some embodiments, subjects are treated with a proteasome inhibitor (e.g., marizomib) at increasing dosages until the subject experiences a CNS-adverse event (e.g., a therapeutic amount). In some embodiments, a dosage can be administered between about 0.25 m/m² and about 2.0 mg/m². For example, an initial dose of marizomib can be about 0.55 mg/m². In some embodiments, the dosage is increased if the subject does not experience a CNS-adverse event at the initial dose (e.g., about 0.55 mg/m²). In some embodiments, if the subject has not experienced a CNS-adverse event, the dosage is increased to about 0.7 mg/m². If the subject still has not experienced a CNS-adverse event, the dosage can be increased to about 0.8 mg/m². If the subject still has not experienced a CNS-adverse event, the dosage can be increased to about 0.9 mg/m². If the subject still has not experienced a CNS-adverse event, the dosage can be increased to about 1.0 mg/m². If the subject still has not experienced a CNS-adverse event, the dosage can be increased to about 1.1 mg/m². If the subject still has not experienced a CNS-adverse event, the dosage can be increased to about 1.2 mg/m². If the subject still has not experienced a CNS-adverse event, the dosage can be increased about 1.5 mg/m². If the subject still has not experienced a CNS-adverse event, the dosage can be increased about 2 mg/m². In some embodiments, the initial dose is about 0.8 mg/m², and the dosage can be increased if the subject does not experience a CNS-adverse event at that dosage. The amount of the proteasome inhibitor (e.g., a therapeutic amount0 is sufficient in the context of the overall therapy regimen being provided to produce a CNS adverse event. The overall therapy regimen can either be a proteasome inhibitor alone (e.g., marizomib) or a proteasome inhibitor in combination with an additional therapeutic agent (e.g., marizomib in combination with bevacizumab).

In some embodiments, subjects (e.g., subjects suffering from grade IV malignant glioma or glioblastoma) are treated with marizomib at a dose of about 0.05 mg/m². In some embodiments, subjects (e.g., subjects suffering from grade IV malignant glioma or glioblastoma) are treated with marizomib at a dose of about 0.1 mg/m². In some embodiments, subjects (e.g., subjects suffering from grade IV malignant glioma or glioblastoma) are treated with marizomib at a dose of about 0.15 mg/m². In some embodiments, subjects (e.g., subjects suffering from grade IV malignant glioma or glioblastoma) are treated with marizomib at a dose of about 0.20 mg/m². In some embodiments, subjects (e.g., subjects suffering from grade IV malignant glioma or glioblastoma) are treated with marizomib at a dose of about 0.25 mg/m². In some embodiments, subjects (e.g., subjects suffering from grade IV malignant glioma or glioblastoma) are treated with marizomib at a dose of about 0.30 mg/m². In some embodiments, subjects (e.g., subjects suffering from grade IV malignant glioma or glioblastoma) are treated with marizomib at a dose of about 0.35 mg/m². In some embodiments, subjects (e.g., subjects suffering from grade IV malignant glioma or glioblastoma) are treated with marizomib at a dose of about 0.40 mg/m². In some embodiments, subjects (e.g., subjects suffering from grade IV malignant glioma or glioblastoma) are treated with marizomib at a dose of about 0.45 mg/m². In some embodiments, subjects (e.g., subjects suffering from grade IV malignant glioma or glioblastoma) are treated with marizomib at a dose of about 0.50 mg/m². In some embodiments, subjects (e.g., subjects suffering from grade IV malignant glioma or glioblastoma) are treated with marizomib at a dose of about 0.55 mg/m². In some embodiments, subjects (e.g., subjects suffering from grade IV malignant glioma or glioblastoma) are treated with marizomib at a dose of about 0.60 mg/m². In some embodiments, subjects (e.g., subjects suffering from grade IV malignant glioma or glioblastoma) are treated with marizomib at a dose of about 0.65 mg/m². In some embodiments, subjects (e.g., subjects suffering from grade IV malignant glioma or glioblastoma) are treated with marizomib at a dose of about 0.70 mg/m². In some embodiments, subjects (e.g., subjects suffering from grade IV malignant glioma or glioblastoma) are treated with marizomib at a dose of about 0.75 mg/m². In some embodiments, subjects (e.g., subjects suffering from grade IV malignant glioma or glioblastoma) are treated with marizomib at a dose of about 0.80 mg/m². In some embodiments, subjects (e.g., subjects suffering from grade IV malignant glioma or glioblastoma) are treated with marizomib at a dose of about 0.85 mg/m². In some embodiments, subjects (e.g., subjects suffering from grade IV malignant glioma or glioblastoma) are treated with marizomib at a dose of about 0.90 mg/m². In some embodiments, subjects (e.g., subjects suffering from grade IV malignant glioma or glioblastoma) are treated with marizomib at a dose of about 0.95 mg/m². In some embodiments, subjects (e.g., subjects suffering from grade IV malignant glioma or glioblastoma) are treated with marizomib at a dose of about 1.00 mg/m². In some embodiments, subjects (e.g., subjects suffering from grade IV malignant glioma or glioblastoma) are treated with marizomib at a dose of about 1.25 mg/m². In some embodiments, subjects (e.g., subjects suffering from grade IV malignant glioma or glioblastoma) are treated with marizomib at a dose of about 1.50 mg/m². In some embodiments, subjects (e.g., subjects suffering from grade IV malignant glioma or glioblastoma) are treated with marizomib at a dose of about 1.75 mg/m². In some embodiments, subjects (e.g., subjects suffering from grade IV malignant glioma or glioblastoma) are treated with marizomib at a dose of about 2.0 mg/m². In some embodiments, subjects (e.g., subjects suffering from grade IV malignant glioma or glioblastoma) are treated with marizomib at a dose of about 2.5 mg/m². In some embodiments, subjects (e.g., subjects suffering from grade IV malignant glioma or glioblastoma) are treated with marizomib at a dose of about 3.0 mg/m². In some embodiments, subjects (e.g., subjects suffering from grade IV malignant glioma or glioblastoma) are treated with marizomib at a dose of about 4.0 mg/m². In some embodiments, subjects (e.g., subjects suffering from grade IV malignant glioma or glioblastoma) are treated with marizomib at a dose of about 5.0 mg/m².

In some embodiments, subjects (e.g., subjects suffering from grade IV malignant glioma or glioblastoma) are treated with marizomib at a dose of about 0.05 mg/m² in combination with bevacizumab. In some embodiments, subjects (e.g., subjects suffering from grade IV malignant glioma or glioblastoma) are treated with marizomib at a dose of about 0.1 mg/m² in combination with bevacizumab. In some embodiments, subjects (e.g., subjects suffering from grade IV malignant glioma or glioblastoma) are treated with marizomib at a dose of about 0.15 mg/m² in combination with bevacizumab. In some embodiments, subjects (e.g., subjects suffering from grade IV malignant glioma or glioblastoma) are treated with marizomib at a dose of about 0.20 mg/m² in combination with bevacizumab. In some embodiments, subjects (e.g., subjects suffering from grade IV malignant glioma or glioblastoma) are treated with marizomib at a dose of about 0.25 mg/m² in combination with bevacizumab. In some embodiments, subjects (e.g., subjects suffering from grade IV malignant glioma or glioblastoma) are treated with marizomib at a dose of about 0.30 mg/m² in combination with bevacizumab. In some embodiments, subjects (e.g., subjects suffering from grade IV malignant glioma or glioblastoma) are treated with marizomib at a dose of about 0.35 mg/m² in combination with bevacizumab. In some embodiments, subjects (e.g., subjects suffering from grade IV malignant glioma or glioblastoma) are treated with marizomib at a dose of about 0.40 mg/m² in combination with bevacizumab. In some embodiments, subjects (e.g., subjects suffering from grade IV malignant glioma or glioblastoma) are treated with marizomib at a dose of about 0.45 mg/m² in combination with bevacizumab. In some embodiments, subjects (e.g., subjects suffering from grade IV malignant glioma or glioblastoma) are treated with marizomib at a dose of about 0.50 mg/m² in combination with bevacizumab. In some embodiments, subjects (e.g., subjects suffering from grade IV malignant glioma or glioblastoma) are treated with marizomib at a dose of about 0.55 mg/m² in combination with bevacizumab. In some embodiments, subjects (e.g., subjects suffering from grade IV malignant glioma or glioblastoma) are treated with marizomib at a dose of about 0.60 mg/m² in combination with bevacizumab. In some embodiments, subjects (e.g., subjects suffering from grade IV malignant glioma or glioblastoma) are treated with marizomib at a dose of about 0.65 mg/m² in combination with bevacizumab. In some embodiments, subjects (e.g., subjects suffering from grade IV malignant glioma or glioblastoma) are treated with marizomib at a dose of about 0.70 mg/m² in combination with bevacizumab. In some embodiments, subjects (e.g., subjects suffering from grade IV malignant glioma or glioblastoma) are treated with marizomib at a dose of about 0.75 mg/m² in combination with bevacizumab. In some embodiments, subjects (e.g., subjects suffering from grade IV malignant glioma or glioblastoma) are treated with marizomib at a dose of about 0.80 mg/m² in combination with bevacizumab. In some embodiments, subjects (e.g., subjects suffering from grade IV malignant glioma or glioblastoma) are treated with marizomib at a dose of about 0.85 mg/m² in combination with bevacizumab. In some embodiments, subjects (e.g., subjects suffering from grade IV malignant glioma or glioblastoma) are treated with marizomib at a dose of about 0.90 mg/m² in combination with bevacizumab. In some embodiments, subjects (e.g., subjects suffering from grade IV malignant glioma or glioblastoma) are treated with marizomib at a dose of about 0.95 mg/m² in combination with bevacizumab. In some embodiments, subjects (e.g., subjects suffering from grade IV malignant glioma or glioblastoma) are treated with marizomib at a dose of about 1.00 mg/m² in combination with bevacizumab. In some embodiments, subjects (e.g., subjects suffering from grade IV malignant glioma or glioblastoma) are treated with marizomib at a dose of about 1.25 mg/m² in combination with bevacizumab. In some embodiments, subjects (e.g., subjects suffering from grade IV malignant glioma or glioblastoma) are treated with marizomib at a dose of about 1.50 mg/m² in combination with bevacizumab. In some embodiments, subjects (e.g., subjects suffering from grade IV malignant glioma or glioblastoma) are treated with marizomib at a dose of about 1.75 mg/m² in combination with bevacizumab. In some embodiments, subjects (e.g., subjects suffering from grade IV malignant glioma or glioblastoma) are treated with marizomib at a dose of about 2.0 mg/m² in combination with bevacizumab. In some embodiments, subjects (e.g., subjects suffering from grade IV malignant glioma or glioblastoma) are treated with marizomib at a dose of about 2.5 mg/m² in combination with bevacizumab. In some embodiments, subjects (e.g., subjects suffering from grade IV malignant glioma or glioblastoma) are treated with marizomib at a dose of about 3.0 mg/m² in combination with bevacizumab. In some embodiments, subjects (e.g., subjects suffering from grade IV malignant glioma or glioblastoma) are treated with marizomib at a dose of about 4.0 mg/m² in combination with bevacizumab. In some embodiments, subjects (e.g., subjects suffering from grade IV malignant glioma or glioblastoma) are treated with marizomib at a dose of about 5.0 mg/m² in combination with bevacizumab.

In some embodiments, subjects (e.g., subjects suffering from grade IV malignant glioma or glioblastoma) are treated with marizomib at a dose of about 0.05 mg/m² in combination with temozolomide. In some embodiments, subjects (e.g., subjects suffering from grade IV malignant glioma or glioblastoma) are treated with marizomib at a dose of about 0.1 mg/m² in combination with temozolomide. In some embodiments, subjects (e.g., subjects suffering from grade IV malignant glioma or glioblastoma) are treated with marizomib at a dose of about 0.15 mg/m² in combination with temozolomide. In some embodiments, subjects (e.g., subjects suffering from grade IV malignant glioma or glioblastoma) are treated with marizomib at a dose of about 0.20 mg/m² in combination with temozolomide. In some embodiments, subjects (e.g., subjects suffering from grade IV malignant glioma or glioblastoma) are treated with marizomib at a dose of about 0.25 mg/m² in combination with temozolomide. In some embodiments, subjects (e.g., subjects suffering from grade IV malignant glioma or glioblastoma) are treated with marizomib at a dose of about 0.30 mg/m² in combination with temozolomide. In some embodiments, subjects (e.g., subjects suffering from grade IV malignant glioma or glioblastoma) are treated with marizomib at a dose of about 0.35 mg/m² in combination with temozolomide. In some embodiments, subjects (e.g., subjects suffering from grade IV malignant glioma or glioblastoma) are treated with marizomib at a dose of about 0.40 mg/m² in combination with temozolomide. In some embodiments, subjects (e.g., subjects suffering from grade IV malignant glioma or glioblastoma) are treated with marizomib at a dose of about 0.45 mg/m² in combination with temozolomide. In some embodiments, subjects (e.g., subjects suffering from grade IV malignant glioma or glioblastoma) are treated with marizomib at a dose of about 0.50 mg/m² in combination with temozolomide. In some embodiments, subjects (e.g., subjects suffering from grade IV malignant glioma or glioblastoma) are treated with marizomib at a dose of about 0.55 mg/m² in combination with temozolomide. In some embodiments, subjects (e.g., subjects suffering from grade IV malignant glioma or glioblastoma) are treated with marizomib at a dose of about 0.60 mg/m² in combination with temozolomide. In some embodiments, subjects (e.g., subjects suffering from grade IV malignant glioma or glioblastoma) are treated with marizomib at a dose of about 0.65 mg/m² in combination with temozolomide. In some embodiments, subjects (e.g., subjects suffering from grade IV malignant glioma or glioblastoma) are treated with marizomib at a dose of about 0.70 mg/m² in combination with temozolomide. In some embodiments, subjects (e.g., subjects suffering from grade IV malignant glioma or glioblastoma) are treated with marizomib at a dose of about 0.75 mg/m² in combination with temozolomide. In some embodiments, subjects (e.g., subjects suffering from grade IV malignant glioma or glioblastoma) are treated with marizomib at a dose of about 0.80 mg/m² in combination with temozolomide. In some embodiments, subjects (e.g., subjects suffering from grade IV malignant glioma or glioblastoma) are treated with marizomib at a dose of about 0.85 mg/m² in combination with temozolomide. In some embodiments, subjects (e.g., subjects suffering from grade IV malignant glioma or glioblastoma) are treated with marizomib at a dose of about 0.90 mg/m² in combination with temozolomide. In some embodiments, subjects (e.g., subjects suffering from grade IV malignant glioma or glioblastoma) are treated with marizomib at a dose of about 0.95 mg/m² in combination with temozolomide. In some embodiments, subjects (e.g., subjects suffering from grade IV malignant glioma or glioblastoma) are treated with marizomib at a dose of about 1.00 mg/m² in combination with temozolomide. In some embodiments, subjects (e.g., subjects suffering from grade IV malignant glioma or glioblastoma) are treated with marizomib at a dose of about 1.25 mg/m² in combination with temozolomide. In some embodiments, subjects (e.g., subjects suffering from grade IV malignant glioma or glioblastoma) are treated with marizomib at a dose of about 1.50 mg/m² in combination with temozolomide. In some embodiments, subjects (e.g., subjects suffering from grade IV malignant glioma or glioblastoma) are treated with marizomib at a dose of about 1.75 mg/m² in combination with temozolomide. In some embodiments, subjects (e.g., subjects suffering from grade IV malignant glioma or glioblastoma) are treated with marizomib at a dose of about 2.0 mg/m² in combination with temozolomide. In some embodiments, subjects (e.g., subjects suffering from grade IV malignant glioma or glioblastoma) are treated with marizomib at a dose of about 2.5 mg/m² in combination with temozolomide. In some embodiments, subjects (e.g., subjects suffering from grade IV malignant glioma or glioblastoma) are treated with marizomib at a dose of about 3.0 mg/m² in combination with temozolomide. In some embodiments, subjects (e.g., subjects suffering from grade IV malignant glioma or glioblastoma) are treated with marizomib at a dose of about 4.0 mg/m² in combination with temozolomide. In some embodiments, subjects (e.g., subjects suffering from grade IV malignant glioma or glioblastoma) are treated with marizomib at a dose of about 5.0 mg/m² in combination with temozolomide. In any of the above-embodiments, temozolomide can be administered at a dose of about 75 mg/m². In any of the above-embodiments, temozolomide can be administered at a dose of between about 150 mg/m² to about 200 mg/m².

In some embodiments, subjects (e.g., subjects suffering from grade IV malignant glioma or glioblastoma) are treated with marizomib at a dose of about 0.05 mg/m² in combination with temozolomide and radiotherapy. In some embodiments, subjects (e.g., subjects suffering from grade IV malignant glioma or glioblastoma) are treated with marizomib at a dose of about 0.1 mg/m² in combination with temozolomide and radiotherapy. In some embodiments, subjects (e.g., subjects suffering from grade IV malignant glioma or glioblastoma) are treated with marizomib at a dose of about 0.15 mg/m² in combination with temozolomide and radiotherapy. In some embodiments, subjects (e.g., subjects suffering from grade IV malignant glioma or glioblastoma) are treated with marizomib at a dose of about 0.20 mg/m² in combination with temozolomide and radiotherapy. In some embodiments, subjects (e.g., subjects suffering from grade IV malignant glioma or glioblastoma) are treated with marizomib at a dose of about 0.25 mg/m² in combination with temozolomide and radiotherapy. In some embodiments, subjects (e.g., subjects suffering from grade IV malignant glioma or glioblastoma) are treated with marizomib at a dose of about 0.30 mg/m² in combination with temozolomide and radiotherapy. In some embodiments, subjects (e.g., subjects suffering from grade IV malignant glioma or glioblastoma) are treated with marizomib at a dose of about 0.35 mg/m² in combination with temozolomide and radiotherapy. In some embodiments, subjects (e.g., subjects suffering from grade IV malignant glioma or glioblastoma) are treated with marizomib at a dose of about 0.40 mg/m² in combination with temozolomide and radiotherapy. In some embodiments, subjects (e.g., subjects suffering from grade IV malignant glioma or glioblastoma) are treated with marizomib at a dose of about 0.45 mg/m² in combination with temozolomide and radiotherapy. In some embodiments, subjects (e.g., subjects suffering from grade IV malignant glioma or glioblastoma) are treated with marizomib at a dose of about 0.50 mg/m² in combination with temozolomide and radiotherapy. In some embodiments, subjects (e.g., subjects suffering from grade IV malignant glioma or glioblastoma) are treated with marizomib at a dose of about 0.55 mg/m² in combination with temozolomide and radiotherapy. In some embodiments, subjects (e.g., subjects suffering from grade IV malignant glioma or glioblastoma) are treated with marizomib at a dose of about 0.60 mg/m² in combination with temozolomide and radiotherapy. In some embodiments, subjects (e.g., subjects suffering from grade IV malignant glioma or glioblastoma) are treated with marizomib at a dose of about 0.65 mg/m² in combination with temozolomide and radiotherapy. In some embodiments, subjects (e.g., subjects suffering from grade IV malignant glioma or glioblastoma) are treated with marizomib at a dose of about 0.70 mg/m² in combination with temozolomide and radiotherapy. In some embodiments, subjects (e.g., subjects suffering from grade IV malignant glioma or glioblastoma) are treated with marizomib at a dose of about 0.75 mg/m² in combination with temozolomide and radiotherapy. In some embodiments, subjects (e.g., subjects suffering from grade IV malignant glioma or glioblastoma) are treated with marizomib at a dose of about 0.80 mg/m² in combination with temozolomide and radiotherapy. In some embodiments, subjects (e.g., subjects suffering from grade IV malignant glioma or glioblastoma) are treated with marizomib at a dose of about 0.85 mg/m² in combination with temozolomide and radiotherapy. In some embodiments, subjects (e.g., subjects suffering from grade IV malignant glioma or glioblastoma) are treated with marizomib at a dose of about 0.90 mg/m² in combination with temozolomide and radiotherapy. In some embodiments, subjects (e.g., subjects suffering from grade IV malignant glioma or glioblastoma) are treated with marizomib at a dose of about 0.95 mg/m² in combination with temozolomide and radiotherapy. In some embodiments, subjects (e.g., subjects suffering from grade IV malignant glioma or glioblastoma) are treated with marizomib at a dose of about 1.00 mg/m² in combination with temozolomide and radiotherapy. In some embodiments, subjects (e.g., subjects suffering from grade IV malignant glioma or glioblastoma) are treated with marizomib at a dose of about 1.25 mg/m² in combination with temozolomide and radiotherapy. In some embodiments, subjects (e.g., subjects suffering from grade IV malignant glioma or glioblastoma) are treated with marizomib at a dose of about 1.50 mg/m² in combination with temozolomide and radiotherapy. In some embodiments, subjects (e.g., subjects suffering from grade IV malignant glioma or glioblastoma) are treated with marizomib at a dose of about 1.75 mg/m² in combination with temozolomide and radiotherapy. In some embodiments, subjects (e.g., subjects suffering from grade IV malignant glioma or glioblastoma) are treated with marizomib at a dose of about 2.0 mg/m² in combination with temozolomide and radiotherapy. In some embodiments, subjects (e.g., subjects suffering from grade IV malignant glioma or glioblastoma) are treated with marizomib at a dose of about 2.5 mg/m² in combination with temozolomide and radiotherapy. In some embodiments, subjects (e.g., subjects suffering from grade IV malignant glioma or glioblastoma) are treated with marizomib at a dose of about 3.0 mg/m² in combination with temozolomide and radiotherapy. In some embodiments, subjects (e.g., subjects suffering from grade IV malignant glioma or glioblastoma) are treated with marizomib at a dose of about 4.0 mg/m² in combination with temozolomide and radiotherapy. In some embodiments, subjects (e.g., subjects suffering from grade IV malignant glioma or glioblastoma) are treated with marizomib at a dose of about 5.0 mg/m² in combination with temozolomide and radiotherapy. In any of the above-embodiments, temozolomide can be administered at a dose of about 75 mg/m² and radiotherapy can be administered at a dose of about 60 Gy. In any of the above-embodiments, temozolomide can be administered at a dose of between about 150 mg/m² to about 200 mg/m² and radiotherapy can be administered at a dose of about 60 Gy.

In some embodiments, marizomib is administered at a dose of about 0.55 mg/m² on days 1, 8, and 15 of a 28-day cycle. In some embodiments, marizomib is administered at a dose of about 0.55 mg/m² on days 1, 8, and 15 of a 28-day cycle in combination with bevacizumab administered at a dose of about 10 mg/kg. In some embodiments, marizomib is administered at a dose of about 0.55 mg/m² on days 1, 8, and 15 of a 28-day cycle in combination with bevacizumab administered at a dose of about 10 mg/kg on days 1 and 14 of a 28-day cycle. In some embodiments, marizomib is administered at a dose of about 0.7 mg/m² on days 1, 8, and 15 of a 28-day cycle. In some embodiments, marizomib is administered at a dose of about 0.7 mg/m² on days 1, 8, and 15 of a 28-day cycle in combination with bevacizumab administered at a dose of about 10 mg/kg. In some embodiments, marizomib is administered at a dose of about 0.7 mg/m² on days 1, 8, and 15 of a 28-day cycle in combination with bevacizumab administered at a dose of about 10 mg/kg on days 1 and 14 of a 28-day cycle. In some embodiments, marizomib is administered at a dose of about 0.8 mg/m² on days 1, 8, and 15 of a 28-day cycle. In some embodiments, marizomib is administered at a dose of about 0.8 mg/m² on days 1, 8, and 15 of a 28-day cycle in combination with bevacizumab administered at a dose of about 10 mg/kg. In some embodiments, marizomib is administered at a dose of about 0.8 mg/m² on days 1, 8, and 15 of a 28-day cycle in combination with bevacizumab administered at a dose of about 10 mg/kg on days 1 and 14 of a 28-day cycle. In some embodiments, marizomib is administered at a dose of about 1.0 mg/m² on days 1, 8, and 15 of a 28-day cycle. In some embodiments, marizomib is administered at a dose of about 1.0 mg/m² on days 1, 8, and 15 of a 28-day cycle in combination with bevacizumab administered at a dose of about 10 mg/kg. In some embodiments, marizomib is administered at a dose of about 1.0 mg/m² on days 1, 8, and 15 of a 28-day cycle in combination with bevacizumab administered at a dose of about 10 mg/kg on days 1 and 14 of a 28-day cycle. In some embodiments, marizomib is administered at a dose of about 1.2 mg/m² on days 1, 8, and 15 of a 28-day cycle. In some embodiments, marizomib is administered at a dose of about 1.2 mg/m² on days 1, 8, and 15 of a 28-day cycle in combination with bevacizumab administered at a dose of about 10 mg/kg. In some embodiments, marizomib is administered at a dose of about 1.2 mg/m² on days 1, 8, and 15 of a 28-day cycle in combination with bevacizumab administered at a dose of about 10 mg/kg on days 1 and 14 of a 28-day cycle.

In some embodiments, marizomib is administered at a dose of about 0.55 mg/m² on days 1, 8, and 15 of a 28-day cycle in combination with temozolomide administered at a dose of about 75 mg/m². In some embodiments, marizomib is administered at a dose of about 0.55 mg/m² on days 1, 8, and 15 of a 28-day cycle in combination with temozolomide administered at a dose of about 75 mg/m² for six weeks. In some embodiments, marizomib is administered at a dose of about 0.55 mg/m² on days 1, 8, and 15 of a 28-day cycle in combination with temozolomide administered at a dose of about 75 mg/m² for about six weeks and in combination with radiotherapy at a dose of about 60 Gy for about 6 weeks. In some embodiments, marizomib is administered at a dose of about 0.55 mg/m² on days 1, 8, and 15 of a 28-day cycle in combination with temozolomide administered at a dose of between about 150 mg/m² to about 200 mg/m² for five consecutive days a week for a 28-day cycle. In some embodiments, marizomib is administered at a dose of about 0.55 mg/m² on days 1, 8, and 15 of a 28-day cycle in combination with temozolomide administered at a dose of between about 150 mg/m² to about 200 mg/m² for five consecutive days a week for a 28-day cycle for 12 cycles. In some embodiments, marizomib is administered at a dose of about 0.55 mg/m² on days 1, 8, and 15 of a 28-day cycle in combination with temozolomide administered at a dose of between about 150 mg/m² to about 200 mg/m² for five consecutive days a week for a 28-day cycle for 12 cycles in combination with radiotherapy at a dose of about 60 Gy for about 6 weeks.

In some embodiments, marizomib is administered at a dose of about 0.7 mg/m² on days 1, 8, and 15 of a 28-day cycle in combination with temozolomide administered at a dose of about 75 mg/m². In some embodiments, marizomib is administered at a dose of about 0.7 mg/m² on days 1, 8, and 15 of a 28-day cycle in combination with temozolomide administered at a dose of about 75 mg/m² for six weeks. In some embodiments, marizomib is administered at a dose of about 0.7 mg/m² on days 1, 8, and 15 of a 28-day cycle in combination with temozolomide administered at a dose of about 75 mg/m² for about six weeks and in combination with radiotherapy at a dose of about 60 Gy for about 6 weeks. In some embodiments, marizomib is administered at a dose of about 0.7 mg/m² on days 1, 8, and 15 of a 28-day cycle in combination with temozolomide administered at a dose of between about 150 mg/m² to about 200 mg/m² for five consecutive days a week for a 28-day cycle. In some embodiments, marizomib is administered at a dose of about 0.7 mg/m² on days 1, 8, and 15 of a 28-day cycle in combination with temozolomide administered at a dose of between about 150 mg/m² to about 200 mg/m² for five consecutive days a week for a 28-day cycle for 12 cycles. In some embodiments, marizomib is administered at a dose of about 0.7 mg/m² on days 1, 8, and 15 of a 28-day cycle in combination with temozolomide administered at a dose of between about 150 mg/m² to about 200 mg/m² for five consecutive days a week for a 28-day cycle for 12 cycles in combination with radiotherapy at a dose of about 60 Gy for about 6 weeks.

In some embodiments, marizomib is administered at a dose of about 0.8 mg/m² on days 1, 8, and 15 of a 28-day cycle in combination with temozolomide administered at a dose of about 75 mg/m². In some embodiments, marizomib is administered at a dose of about 0.8 mg/m² on days 1, 8, and 15 of a 28-day cycle in combination with temozolomide administered at a dose of about 75 mg/m² for six weeks. In some embodiments, marizomib is administered at a dose of about 0.8 mg/m² on days 1, 8, and 15 of a 28-day cycle in combination with temozolomide administered at a dose of about 75 mg/m² for about six weeks and in combination with radiotherapy at a dose of about 60 Gy for about 6 weeks. In some embodiments, marizomib is administered at a dose of about 0.8 mg/m² on days 1, 8, and 15 of a 28-day cycle in combination with temozolomide administered at a dose of between about 150 mg/m² to about 200 mg/m² for five consecutive days a week for a 28-day cycle. In some embodiments, marizomib is administered at a dose of about 0.8 mg/m² on days 1, 8, and 15 of a 28-day cycle in combination with temozolomide administered at a dose of between about 150 mg/m² to about 200 mg/m² for five consecutive days a week for a 28-day cycle for 12 cycles. In some embodiments, marizomib is administered at a dose of about 0.8 mg/m² on days 1, 8, and 15 of a 28-day cycle in combination with temozolomide administered at a dose of between about 150 mg/m² to about 200 mg/m² for five consecutive days a week for a 28-day cycle for 12 cycles in combination with radiotherapy at a dose of about 60 Gy for about 6 weeks.

In some embodiments, marizomib is administered at a dose of about 1.0 mg/m² on days 1, 8, and 15 of a 28-day cycle in combination with temozolomide administered at a dose of about 75 mg/m². In some embodiments, marizomib is administered at a dose of about 1.0 mg/m² on days 1, 8, and 15 of a 28-day cycle in combination with temozolomide administered at a dose of about 75 mg/m² for six weeks. In some embodiments, marizomib is administered at a dose of about 1.0 mg/m² on days 1, 8, and 15 of a 28-day cycle in combination with temozolomide administered at a dose of about 75 mg/m² for about six weeks and in combination with radiotherapy at a dose of about 60 Gy for about 6 weeks. In some embodiments, marizomib is administered at a dose of about 1.0 mg/m² on days 1, 8, and 15 of a 28-day cycle in combination with temozolomide administered at a dose of between about 150 mg/m² to about 200 mg/m² for five consecutive days a week for a 28-day cycle. In some embodiments, marizomib is administered at a dose of about 1.0 mg/m² on days 1, 8, and 15 of a 28-day cycle in combination with temozolomide administered at a dose of between about 150 mg/m² to about 200 mg/m² for five consecutive days a week for a 28-day cycle for 12 cycles. In some embodiments, marizomib is administered at a dose of about 1.0 mg/m² on days 1, 8, and 15 of a 28-day cycle in combination with temozolomide administered at a dose of between about 150 mg/m² to about 200 mg/m² for five consecutive days a week for a 28-day cycle for 12 cycles in combination with radiotherapy at a dose of about 60 Gy for about 6 weeks.

In some embodiments, marizomib is administered at a dose of about 1.2 mg/m² on days 1, 8, and 15 of a 28-day cycle in combination with temozolomide administered at a dose of about 75 mg/m². In some embodiments, marizomib is administered at a dose of about 1.2 mg/m² on days 1, 8, and 15 of a 28-day cycle in combination with temozolomide administered at a dose of about 75 mg/m² for six weeks. In some embodiments, marizomib is administered at a dose of about 1.2 mg/m² on days 1, 8, and 15 of a 28-day cycle in combination with temozolomide administered at a dose of about 75 mg/m² for about six weeks and in combination with radiotherapy at a dose of about 60 Gy for about 6 weeks. In some embodiments, marizomib is administered at a dose of about 1.2 mg/m² on days 1, 8, and 15 of a 28-day cycle in combination with temozolomide administered at a dose of between about 150 mg/m² to about 200 mg/m² for five consecutive days a week for a 28-day cycle. In some embodiments, marizomib is administered at a dose of about 1.2 mg/m² on days 1, 8, and 15 of a 28-day cycle in combination with temozolomide administered at a dose of between about 150 mg/m² to about 200 mg/m² for five consecutive days a week for a 28-day cycle for 12 cycles. In some embodiments, marizomib is administered at a dose of about 1.2 mg/m² on days 1, 8, and 15 of a 28-day cycle in combination with temozolomide administered at a dose of between about 150 mg/m² to about 200 mg/m² for five consecutive days a week for a 28-day cycle for 12 cycles in combination with radiotherapy at a dose of about 60 Gy for about 6 weeks.

In some embodiments, bevacizumab is administered at a dose of about 1 mg/kg (e.g., on days 1 and 14 of a 28-day cycle). In some embodiments, bevacizumab is administered at a dose of about 2 mg/kg (e.g., on days 1 and 14 of a 28-day cycle). In some embodiments, bevacizumab is administered at a dose of about 3 mg/kg (e.g., on days 1 and 14 of a 28-day cycle). In some embodiments, bevacizumab is administered at a dose of about 4 mg/kg (e.g., on days 1 and 14 of a 28-day cycle). In some embodiments, bevacizumab is administered at a dose of about 5 mg/kg (e.g., on days 1 and 14 of a 28-day cycle). In some embodiments, bevacizumab is administered at a dose of about 6 mg/kg (e.g., on days 1 and 14 of a 28-day cycle). In some embodiments, bevacizumab is administered at a dose of about 7 mg/kg (e.g., on days 1 and 14 of a 28-day cycle). In some embodiments, bevacizumab is administered at a dose of about 8 mg/kg (e.g., on days 1 and 14 of a 28-day cycle). In some embodiments, bevacizumab is administered at a dose of about 9 mg/kg (e.g., on days 1 and 14 of a 28-day cycle). In some embodiments, bevacizumab is administered at a dose of about 10 mg/kg (e.g., on days 1 and 14 of a 28-day cycle). In some embodiments, bevacizumab is administered at a dose of about 11 mg/kg (e.g., on days 1 and 14 of a 28-day cycle). In some embodiments, bevacizumab is administered at a dose of about 12 mg/kg (e.g., on days 1 and 14 of a 28-day cycle). In some embodiments, bevacizumab is administered at a dose of about 13 mg/kg (e.g., on days 1 and 14 of a 28-day cycle). In some embodiments, bevacizumab is administered at a dose of about 14 mg/kg (e.g., on days 1 and 14 of a 28-day cycle). In some embodiments, bevacizumab is administered at a dose of about 15 mg/kg (e.g., on days 1 and 14 of a 28-day cycle). In some embodiments, bevacizumab is administered at a dose of about 20 mg/kg (e.g., on days 1 and 14 of a 28-day cycle). In some embodiments, bevacizumab is administered at a dose of about 25 mg/kg (e.g., on days 1 and 14 of a 28-day cycle).

In some embodiments, temozolomide is administered at a dose of about 10 mg/kg (e.g., on five consecutive days a week for a 28-day cycle). In some embodiments, temozolomide is administered at a dose of about 15 mg/kg (e.g., on five consecutive days a week for a 28-day cycle). In some embodiments, temozolomide is administered at a dose of about 20 mg/kg (e.g., on five consecutive days a week for a 28-day cycle). In some embodiments, temozolomide is administered at a dose of about 25 mg/kg (e.g., on five consecutive days a week for a 28-day cycle). In some embodiments, temozolomide is administered at a dose of about 30 mg/kg (e.g., on five consecutive days a week for a 28-day cycle). In some embodiments, temozolomide is administered at a dose of about 35 mg/kg (e.g., on five consecutive days a week for a 28-day cycle). In some embodiments, temozolomide is administered at a dose of about 40 mg/kg (e.g., on five consecutive days a week for a 28-day cycle). In some embodiments, temozolomide is administered at a dose of about 45 mg/kg (e.g., on five consecutive days a week for a 28-day cycle). In some embodiments, temozolomide is administered at a dose of about 50 mg/kg (e.g., on five consecutive days a week for a 28-day cycle). In some embodiments, temozolomide is administered at a dose of about 55 mg/kg (e.g., on five consecutive days a week for a 28-day cycle). In some embodiments, temozolomide is administered at a dose of about 60 mg/kg (e.g., on five consecutive days a week for a 28-day cycle).

In some embodiments, temozolomide is administered at a dose of about 65 mg/kg (e.g., on five consecutive days a week for a 28-day cycle). In some embodiments, temozolomide is administered at a dose of about 70 mg/kg (e.g., on five consecutive days a week for a 28-day cycle). In some embodiments, temozolomide is administered at a dose of about 75 mg/kg (e.g., on five consecutive days a week for a 28-day cycle). In some embodiments, temozolomide is administered at a dose of about 80 mg/kg (e.g., on five consecutive days a week for a 28-day cycle). In some embodiments, temozolomide is administered at a dose of about 85 mg/kg (e.g., on five consecutive days a week for a 28-day cycle). In some embodiments, temozolomide is administered at a dose of about 90 mg/kg (e.g., on five consecutive days a week for a 28-day cycle). In some embodiments, temozolomide is administered at a dose of about 95 mg/kg (e.g., on five consecutive days a week for a 28-day cycle). In some embodiments, temozolomide is administered at a dose of about 100 mg/kg (e.g., on five consecutive days a week for a 28-day cycle). In some embodiments, temozolomide is administered at a dose of about 110 mg/kg (e.g., on five consecutive days a week for a 28-day cycle). In some embodiments, temozolomide is administered at a dose of about 120 mg/kg (e.g., on five consecutive days a week for a 28-day cycle). In some embodiments, temozolomide is administered at a dose of about 130 mg/kg (e.g., on five consecutive days a week for a 28-day cycle). In some embodiments, temozolomide is administered at a dose of about 140 mg/kg (e.g., on five consecutive days a week for a 28-day cycle). In some embodiments, temozolomide is administered at a dose of about 150 mg/kg (e.g., on five consecutive days a week for a 28-day cycle). In some embodiments, temozolomide is administered at a dose of about 200 mg/kg (e.g., on five consecutive days a week for a 28-day cycle). In some embodiments, temozolomide is administered at a dose of about 250 mg/kg (e.g., on five consecutive days a week for a 28-day cycle). In some embodiments, temozolomide is administered at a dose of about 300 mg/kg (e.g., on five consecutive days a week for a 28-day cycle). In some embodiments, temozolomide is administered at a dose of about 350 mg/kg (e.g., on five consecutive days a week for a 28-day cycle). In some embodiments, temozolomide is administered at a dose of about 400 mg/kg (e.g., on five consecutive days a week for a 28-day cycle). In some embodiments, temozolomide is administered at a dose of about 450 mg/kg (e.g., on five consecutive days a week for a 28-day cycle). In some embodiments, temozolomide is administered at a dose of about 500 mg/kg (e.g., on five consecutive days a week for a 28-day cycle).

In some embodiments, radiotherapy is administered at a dose of about 5 Gy (e.g., for about 1, 2, 3, 4, 5, 6, 7, 8, 9, or 10 weeks). In some embodiments, radiotherapy is administered at a dose of about 10 Gy (e.g., for about 1, 2, 3, 4, 5, 6, 7, 8, 9, or 10 weeks). In some embodiments, radiotherapy is administered at a dose of about 15 Gy (e.g., for about 1, 2, 3, 4, 5, 6, 7, 8, 9, or 10 weeks). In some embodiments, radiotherapy is administered at a dose of about 20 Gy (e.g., for about 1, 2, 3, 4, 5, 6, 7, 8, 9, or 10 weeks). In some embodiments, radiotherapy is administered at a dose of about 25 Gy (e.g., for about 1, 2, 3, 4, 5, 6, 7, 8, 9, or 10 weeks). In some embodiments, radiotherapy is administered at a dose of about 30 Gy (e.g., for about 1, 2, 3, 4, 5, 6, 7, 8, 9, or 10 weeks). In some embodiments, radiotherapy is administered at a dose of about 35 Gy (e.g., for about 1, 2, 3, 4, 5, 6, 7, 8, 9, or 10 weeks). In some embodiments, radiotherapy is administered at a dose of about 40 Gy (e.g., for about 1, 2, 3, 4, 5, 6, 7, 8, 9, or 10 weeks). In some embodiments, radiotherapy is administered at a dose of about 45 Gy (e.g., for about 1, 2, 3, 4, 5, 6, 7, 8, 9, or 10 weeks). In some embodiments, radiotherapy is administered at a dose of about 50 Gy (e.g., for about 1, 2, 3, 4, 5, 6, 7, 8, 9, or 10 weeks). In some embodiments, radiotherapy is administered at a dose of about 55 Gy (e.g., for about 1, 2, 3, 4, 5, 6, 7, 8, 9, or 10 weeks). In some embodiments, radiotherapy is administered at a dose of about 60 Gy (e.g., for about 1, 2, 3, 4, 5, 6, 7, 8, 9, or 10 weeks). In some embodiments, radiotherapy is administered at a dose of about 65 Gy (e.g., for about 1, 2, 3, 4, 5, 6, 7, 8, 9, or 10 weeks). In some embodiments, radiotherapy is administered at a dose of about 70 Gy (e.g., for about 1, 2, 3, 4, 5, 6, 7, 8, 9, or 10 weeks). In some embodiments, radiotherapy is administered at a dose of about 75 Gy (e.g., for about 1, 2, 3, 4, 5, 6, 7, 8, 9, or 10 weeks). In some embodiments, radiotherapy is administered at a dose of about 80 Gy (e.g., for about 1, 2, 3, 4, 5, 6, 7, 8, 9, or 10 weeks). In some embodiments, radiotherapy is administered at a dose of about 85 Gy (e.g., for about 1, 2, 3, 4, 5, 6, 7, 8, 9, or 10 weeks). In some embodiments, radiotherapy is administered at a dose of about 90 Gy (e.g., for about 1, 2, 3, 4, 5, 6, 7, 8, 9, or 10 weeks). In some embodiments, radiotherapy is administered at a dose of about 95 Gy (e.g., for about 1, 2, 3, 4, 5, 6, 7, 8, 9, or 10 weeks). In some embodiments, radiotherapy is administered at a dose of about 100 Gy (e.g., for about 1, 2, 3, 4, 5, 6, 7, 8, 9, or 10 weeks). In some embodiments, radiotherapy is administered at a dose of about 110 Gy (e.g., for about 1, 2, 3, 4, 5, 6, 7, 8, 9, or 10 weeks). In some embodiments, radiotherapy is administered at a dose of about 120 Gy (e.g., for about 1, 2, 3, 4, 5, 6, 7, 8, 9, or 10 weeks). In some embodiments, radiotherapy is administered at a dose of about 130 Gy (e.g., for about 1, 2, 3, 4, 5, 6, 7, 8, 9, or 10 weeks). In some embodiments, radiotherapy is administered at a dose of about 140 Gy (e.g., for about 1, 2, 3, 4, 5, 6, 7, 8, 9, or 10 weeks). In some embodiments, radiotherapy is administered at a dose of about 150 Gy (e.g., for about 1, 2, 3, 4, 5, 6, 7, 8, 9, or 10 weeks). In some embodiments, radiotherapy is administered at a dose of about 200 Gy (e.g., for about 1, 2, 3, 4, 5, 6, 7, 8, 9, or 10 weeks). In some embodiments, radiotherapy is administered at a dose of about 250 Gy (e.g., for about 1, 2, 3, 4, 5, 6, 7, 8, 9, or 10 weeks). In some embodiments, radiotherapy is administered at a dose of about 300 Gy (e.g., for about 1, 2, 3, 4, 5, 6, 7, 8, 9, or 10 weeks). In some embodiments, radiotherapy is administered at a dose of about 350 Gy (e.g., for about 1, 2, 3, 4, 5, 6, 7, 8, 9, or 10 weeks). In some embodiments, radiotherapy is administered at a dose of about 400 Gy (e.g., for about 1, 2, 3, 4, 5, 6, 7, 8, 9, or 10 weeks). In some embodiments, radiotherapy is administered at a dose of about 450 Gy (e.g., for about 1, 2, 3, 4, 5, 6, 7, 8, 9, or 10 weeks). In some embodiments, radiotherapy is administered at a dose of about 500 Gy (e.g., for about 1, 2, 3, 4, 5, 6, 7, 8, 9, or 10 weeks).

Table 5 below shows the CNS adverse events at all grades by dose of marizomib for patients treated with marizomib. Table 6 shows the CNS adverse events excluding fatigue.

TABLE 5
CNS Adverse Events by Dose

| | | Dose (mg/m²) and | | |
|---|---|---|---|---|
| | # Patients | Number of patients | | |
| | TOTAL (36) | 0.55 (6) | 0.7 (3) | 0.8 (27) |
| Agitation | 3 | | | 3 |
| Anxiety | 6 | 3 | | 3 |
| Aphasia | 6 | | 1 | 5 |
| Apraxia | 1 | | | 1 |
| Ataxia | 8 | | 1 | 7 |
| Balance disorder | 1 | | | 1 |
| Cognitive disorder | 0 | | | |
| Concentration Impairment | 0 | | | |
| Confusional State | 10 | 1 | | 9 |
| Convulsion | 7 | 1 | 1 | 5 |
| Delirium | 0 | | | |
| Delusion | 1 | | | 1 |
| Depressed Level of | 1 | | | 1 |
| Consciousness | | | | |
| Depression, Depressed Mood | 4 | 2 | | 2 |
| Dizziness | 10 | 1 | 1 | 8 |
| Dysarthria | 7 | 1 | | 6 |
| Facial nerve disorder | 2 | | | 2 |
| Facial paresis | 3 | | | 3 |
| Fall | 10 | 3 | | 7 |
| Fatigue | 26 | 3 | 2 | 21 |
| Gait disturbance | 5 | | 1 | 4 |
| Hallucination | 13 | 1 | | 12 |
| Insomnia | 4 | 1 | | 3 |
| Intention tremor | 1 | | | 1 |
| Irritability | 1 | | | 1 |
| Memory impairment | 7 | | | 7 |
| Mental status changes | 1 | | | 1 |
| Personality change | 1 | | | 1 |
| Psychotic disorder | 1 | | | 1 |
| Pyramidal tract syndrome | 3 | | | 3 |
| Somnolence | 2 | | | 2 |
| Suicidal ideation | 1 | | | 1 |
| Tremor | 1 | | | 1 |
| Trigeminal nerve disorder | 1 | | | 1 |
| Vertigo | 0 | | | |

TABLE 6
CNS Adverse Events by Patient (Excluding Fatigue)

| Pt. | | | | | | | | | | |
|---|---|---|---|---|---|---|---|---|---|---|
| No. | Anxiety | Aphasia | Ataxia | Confusion | Convulsions | Dizziness | Dysarthria | Fall | Gait | Memory |
| 0103 | X | | | X | X | | | X | | |
| 0104 | | | | | | | X | X | | |
| 0105 | X | | | | | X | | | | |
| 0106 | | | | | | | | X | | |
| 0107 | X | | | | | | | | | |
| 0201 | | | X | | | X | | | X | |
| 0203 | | X | | | X | | | | | |
| 0301 | X | X | | | | | X | | | X |
| 0302 | | | | X | | X | | | | |
| 0303 | | | X | X | X | | X | X | | X |
| 0401 | | X | X | | | | | X | | |
| 0402 | | | | X | | | | | | |
| 0404 | | | | X | X | | | | | X |
| 0407 | | | | X | | X | | | | |
| 0408 | | | X | X | | X | X | X | | |
| 0410 | X | | | | | X | X | X | | |
| 0411 | | | X | | | | X | | | |
| 0412 | | X | X | X | X | X | | | X | |
| 0413 | | | | X | | | | | | |
| 0414 | X | X | | | X | | | | X | X |
| 0416 | | | X | | | | | | | |
| 0417 | | | | | | | | X | X | X |
| 0418 | | | | | | X | | | X | X |
| 0419 | | | X | | | X | | X | | |
| 0420 | | | | X | | X | X | X | | |
| 0423 | | X | | | X | | | | | |
| 0424 | | | | | | | | | | X |

### Hallucinations

Without wishing to be bound by theory, an association between clinical activity and hallucinations was observed. Without wishing to be bound by theory, the time of a CNS adverse event ranged between 1 and 17 doses of marizomib, with a median of about 5 doses. Without wishing to be bound by theory, the time of onset of CNS adverse events ranged from the day of treatment to six days post-marizomib infusion, with a median of one day after infusion.

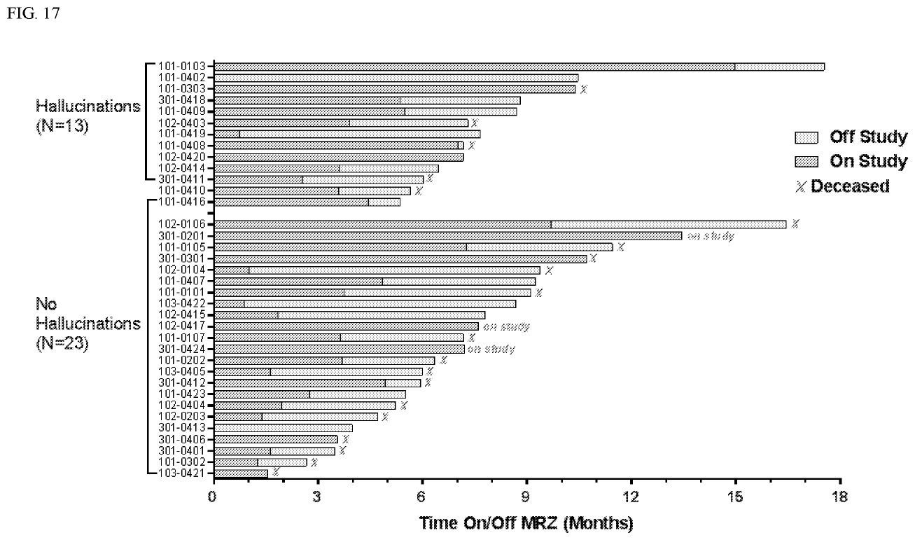

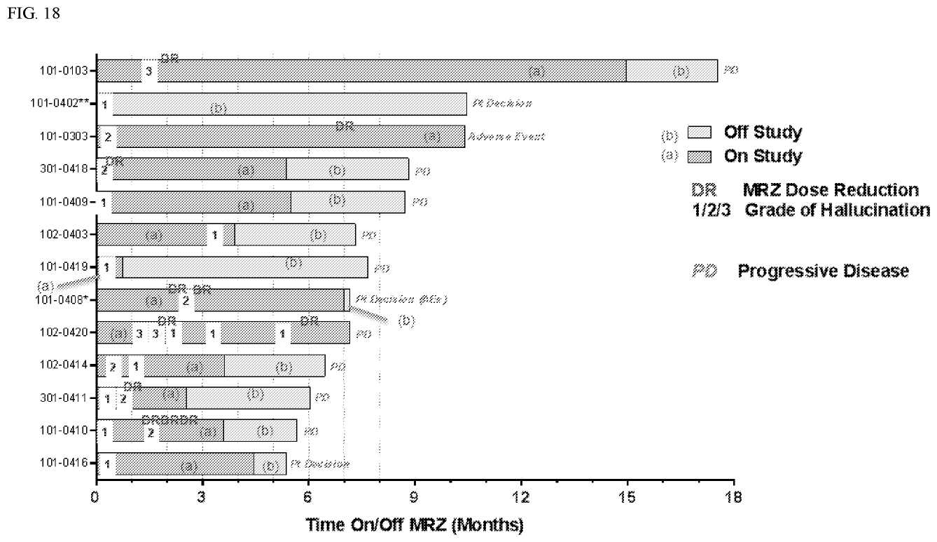

Set forth in FIG. 17 is a plot of the number of patients who did and did not experience hallucination as a function of time. Set forth in FIG. 18 is a plot of the timing of hallucinations and dose reductions.

Table 7 shows treatment responses for subjects who did and did not experience hallucinations. As set forth in Table 7, more patients who experienced hallucination expensed a partial response or better compared with patients who did not experience hallucination.

TABLE 7
Response of Patients as a Function of Hallucination

| | | |
|---|---|---|
| Best Response | Hallucinators (13) | Non Hallucinators (23) |

| | | | | |
|---|---|---|---|---|
| ≥Partial Response | 7 | (54%) | 8 | (35%) |
| Stable disease | 4 | (31%) | 8 | (35%) |
| Progressive disease | 1 | (8%) | 5 | (22%) |
| Not Evaluated | 1 | (8%) | 2 | (9%) |

Table 8 shows the timing of hallucinations and marizomib dose reductions in patients.

TABLE 8
Timing of Hallucinations and Marizomib Dose Reductions

| | Hallucination | Time to | | | # MRZ Doses | Dose | Duration |
|---|---|---|---|---|---|---|---|
| Patient | Timing | Onset | | Grade | Prior to Event | Reduced? | (Days) |
| 0103 | Cycle 2 Day 15 | 2 | days | 3 | 5 | Cycle 3 Day 1 | 1 |
| 0303 | Cycle 1 Day 8 | Same day | | 2 | 3 | Cycle 8 Day 1 | |
| 0402 | Cycle 1 Day 8 | 2 | days | 1 | 2 | No | |
| 0408* | Cycle 3 Day 15 | 4 | days | 2 | 9 | Cycle 3 Day 8 & | <1 |
| | | | | | | Cycle 4 Day 1 | |
| 0409 | Cycle 1 Day 1 | Same day | | 1 | 1 | No | |
| 0410 | Cycle 1 Day 1 | Same day | | 1 | 1 | No | 28 |
| 0416 | Cycle 1 Day 8 | Same day | | 1 | 2 | Cycle 2 Day 15, | |
| | Cycle 2 Day 15 | 1 | day | 2 | 6 | Cycle 3 Day 1, | 20 |
| | | | | | | Cycle 3 Day 15 | |
| 0419 | Cycle 1 Day 8 | 1 | day | 1 | 2 | No | |
| 0403 | Cycle 4 Day 8 | 1 | day | 1 | 14 | No | 1 |
| 0414 | Cycle 1 Day 15 | 4 | days | 2 | 3 | No | 7 |
| | Cycle 2 Day 8 | 1 | day | 1 | 5 | | 7 |
| 0420 | Cycle 2 Day 8 | Same day | | 3 | 5 | Cycle 3 Day 1 & | 3 |
| | Cycle 2 Day 15 | 1 | day | 3 | 6 | Cycle 7 Day 1 | 12 |
| | | | | 1 | 7 | | 1 |
| | Cycle 3 Day 1 | Same day | | 1 | 11 | | 1 |
| | Cycle 4 Day 8 | 6 | days | 1 | 17 | | |
| | Cycle 6 Day 8 | Same day | | | | | |
| 0411 | Cycle 1 Day 8 | 1 | day | 1 | 2 | Cycle 2 Day 1 | 1 |
| | Cycle 1 Day 15 | 1 | day | 2 | 3 | | 3 |

| | | | | | |
|---|---|---|---|---|---|
| 0418 | Cycle 1 Day 1 | Same day | 2 | 1 | Cycle 1 Day 15 |

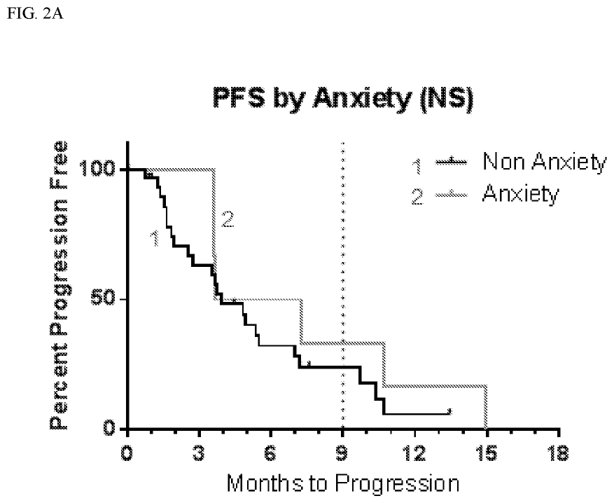

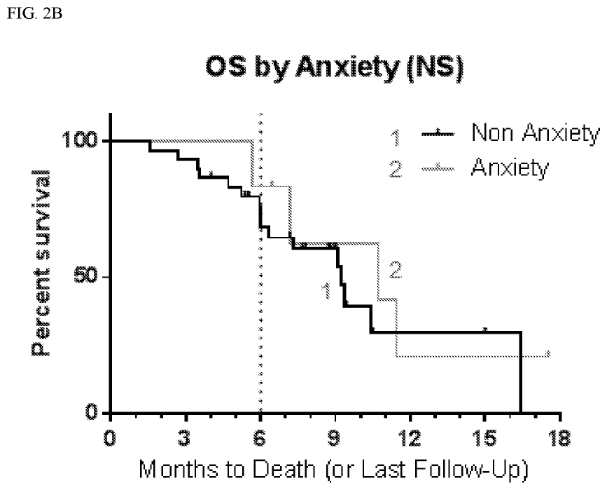

Set forth in FIGS. 1-18 is a summary of the progression free survival (PFS) and overall survival (OS) in patients as a function of CNS adverse events. FIGS. 1A an 1B show the PFS and OS (respectively) for patients who experienced at least one of ataxia, gait disturbance, fall, dysarthria, dizziness, including balance disorders, and hallucinations. FIGS. 1C and 1D show the PFS and OS (respectively) for patients who experienced at least one of ataxia, gait disturbance, fall, dysarthria, and dizziness, including balance disorders). FIGS. 2A-16B show plots of PFS and OS for individual CNS adverse events in patients.

FIG. 1A shows progression free survival (PFS) according to whether patients suffered ataxia, gait disturbance, fall, dysarthria, dizziness, including balance disorders, and hallucinations. FIG. 1B shows overall survival (OS) according to whether patients suffered ataxia, gait disturbance, fall, dysarthria, dizziness, including balance disorders, and hallucinations. FIG. 1C shows progression free survival (PFS) according to whether patients suffered ataxia, gait disturbance, fall, dysarthria, and dizziness, including balance disorders. FIG. 1D shows overall survival (OS) according to whether patients suffered ataxia, gait disturbance, fall, dysarthria, and dizziness, including balance disorders.

FIG. 2A shows progression free survival (PFS) according to whether patients suffered anxiety. FIG. 2B shows overall survival (OS) according to whether patients suffered anxiety.

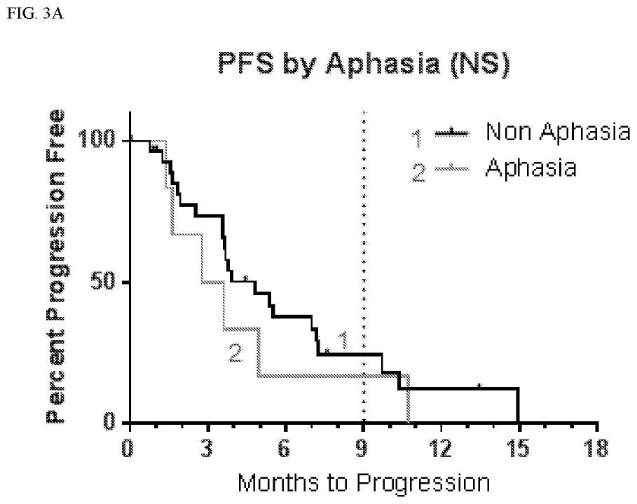

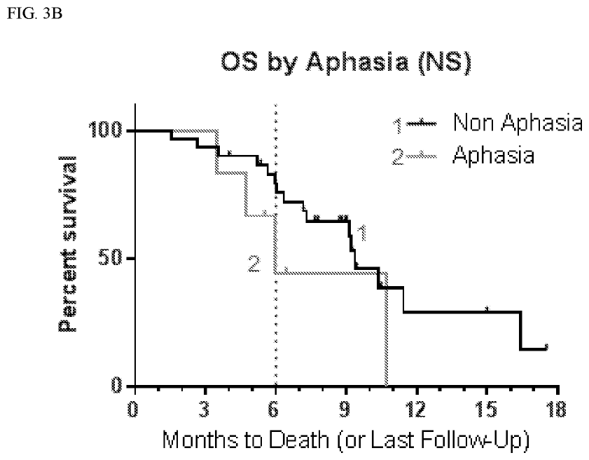

FIG. 3A shows progression free survival (PFS) according to whether patients suffered aphasia. FIG. 3B shows overall survival (OS) according to whether patients suffered aphasia.

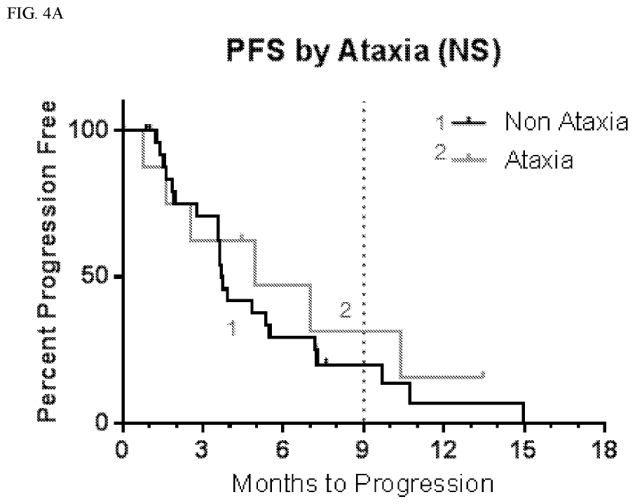

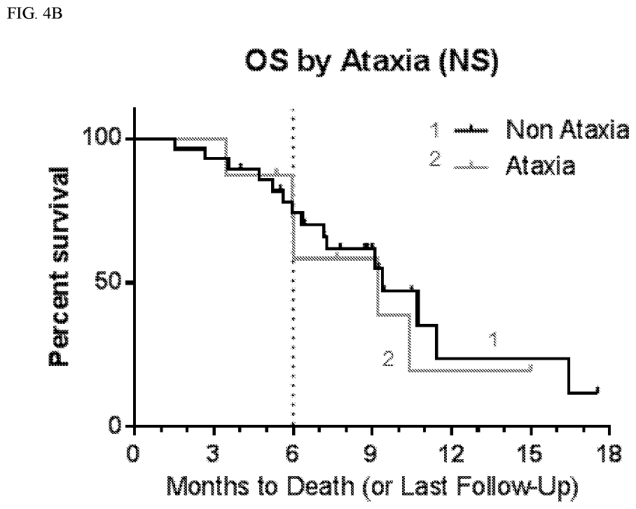

FIG. 4A shows progression free survival (PFS) according to whether patients suffered ataxia. FIG. 4B shows overall survival (OS) according to whether patients suffered ataxia.

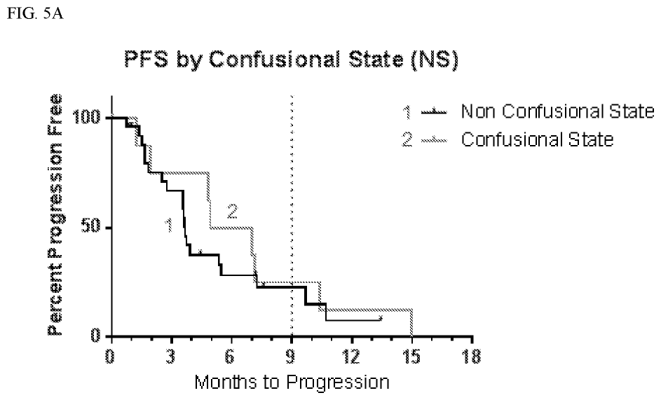

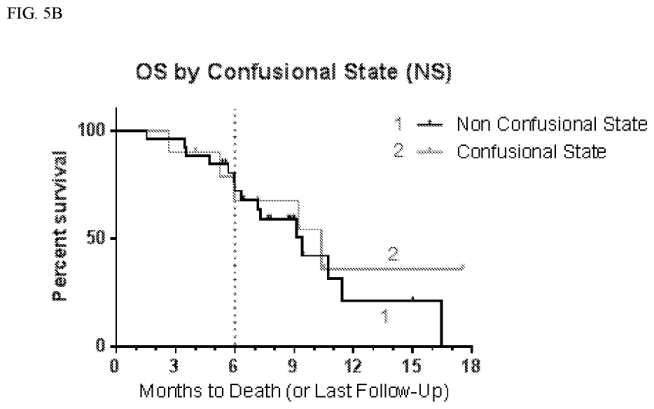

FIG. 5A shows progression free survival (PFS) according to whether patients suffered confusional state. FIG. 5B shows overall survival (OS) according to whether patients suffered confusional state.

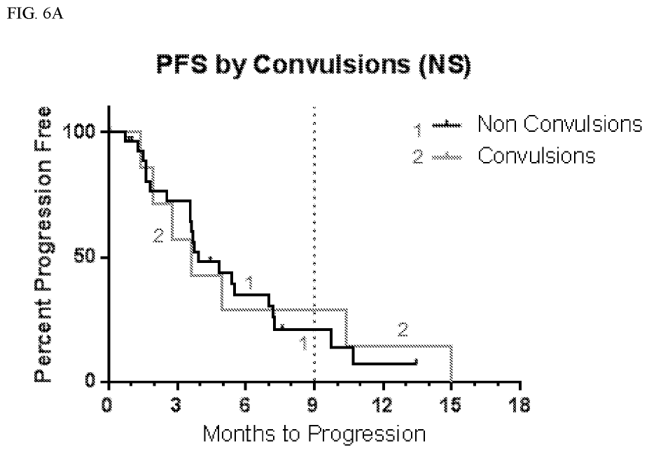

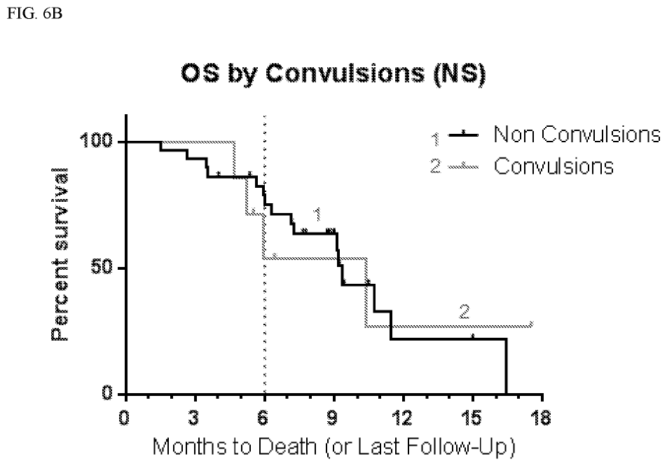

FIG. 6A shows progression free survival (PFS) according to whether patients suffered convulsions. FIG. 6B shows overall survival (OS) according to whether patients suffered convulsions.

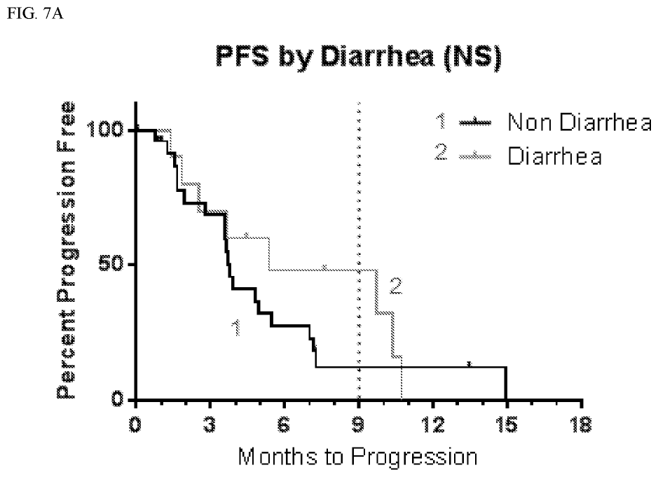

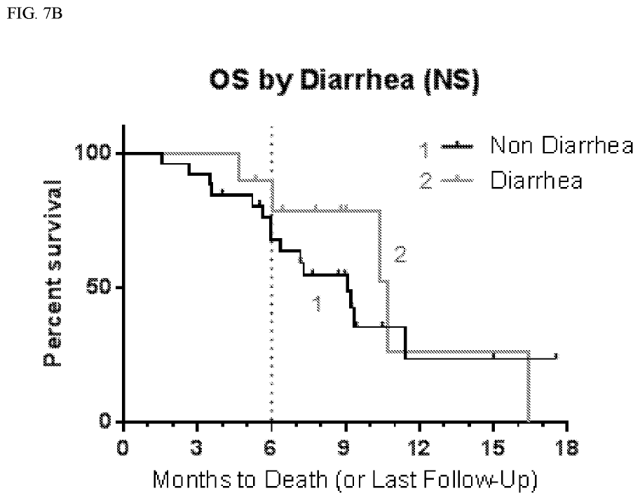

FIG. 7A shows progression free survival (PFS) according to whether patients suffered diarrhea. FIG. 7B shows overall survival (OS) according to whether patients suffered diarrhea.

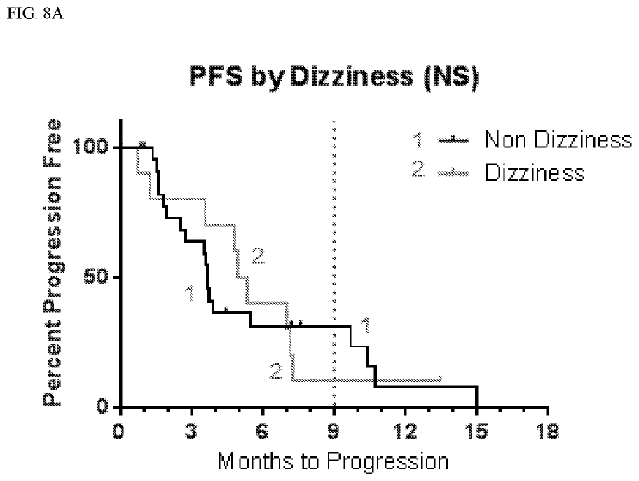

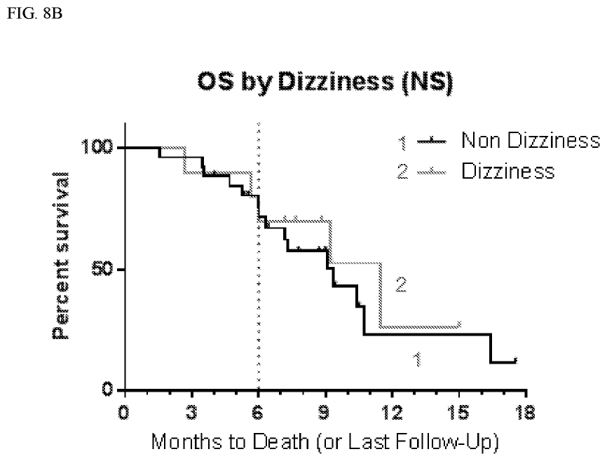

FIG. 8A shows progression free survival (PFS) according to whether patients suffered dizziness. FIG. 8B shows overall survival (OS) according to whether patients suffered dizziness.

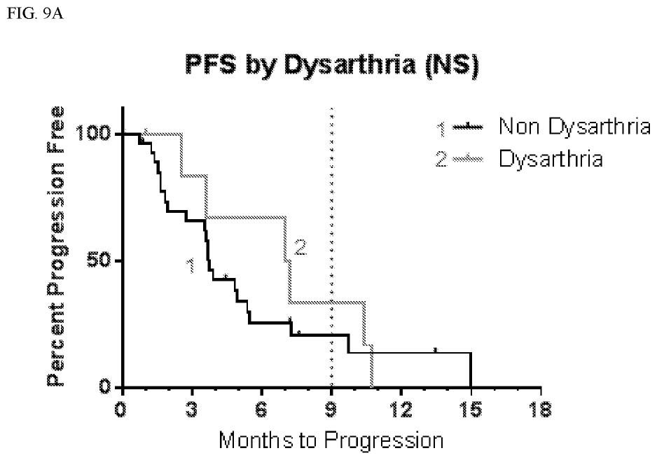

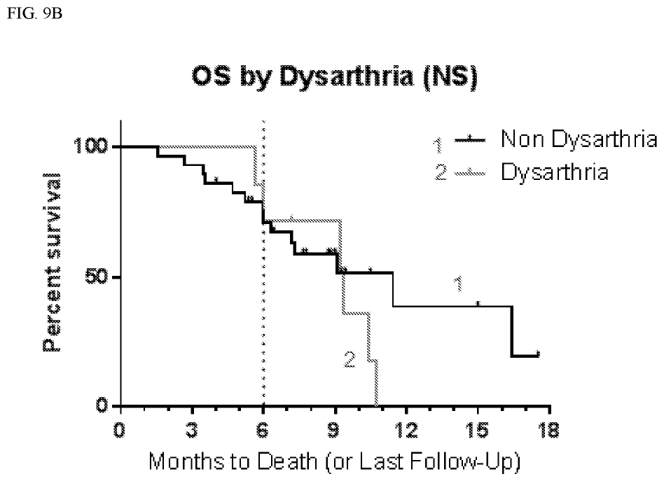

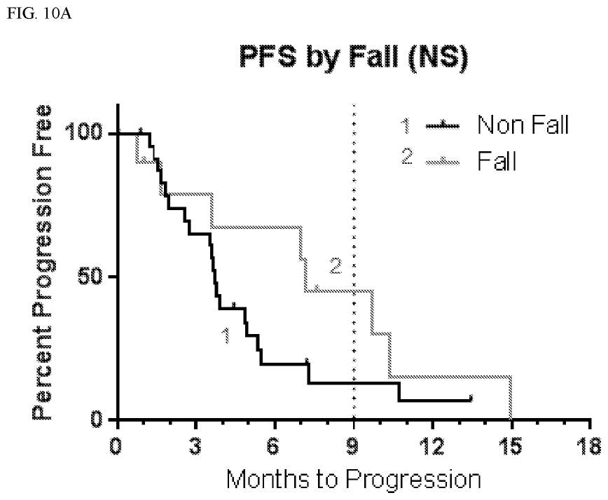

FIG. 9A shows progression free survival (PFS) according to whether patients suffered dysarthria. FIG. 9B shows overall survival (OS) according to whether patients suffered dysarthria.

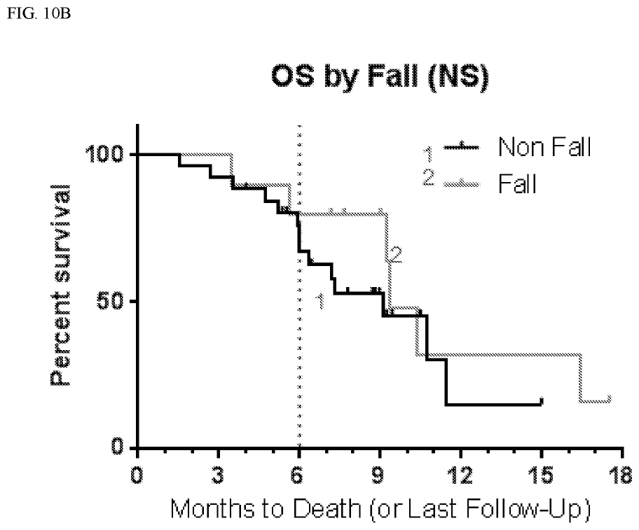

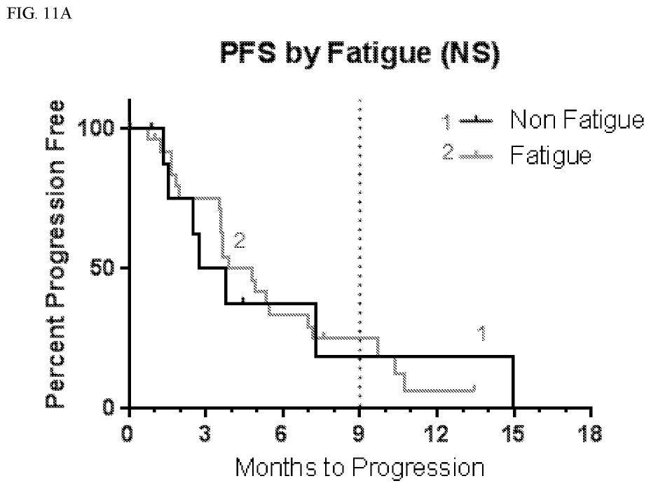

FIG. 10A shows progression free survival (PFS) according to whether patients suffered fall. FIG. 10B shows overall survival (OS) according to whether patients suffered fall.

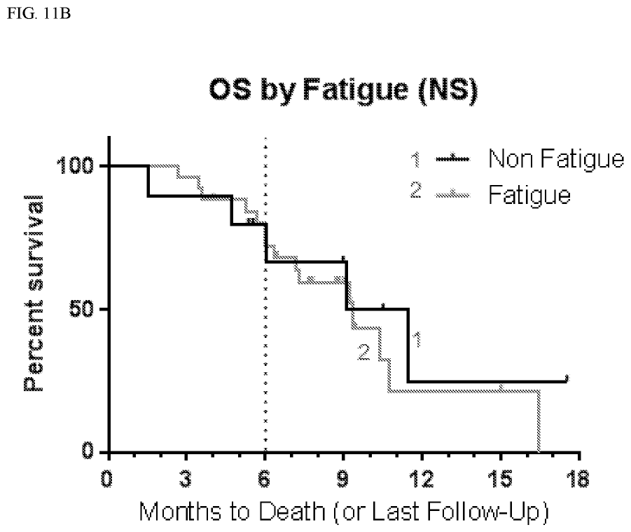

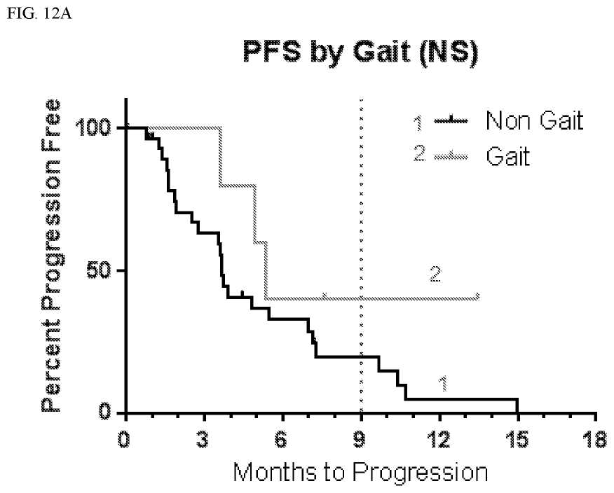

FIG. 11A shows progression free survival (PFS) according to whether patients suffered fatigue. FIG. 11B shows overall survival (OS) according to whether patients suffered fatigue.

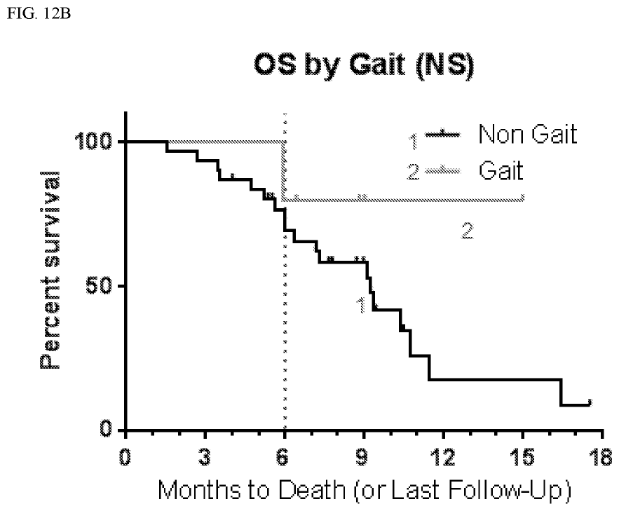

FIG. 12A shows progression free survival (PFS) according to whether patients suffered gait disturbance. FIG. 12B shows overall survival (OS) according to whether patients suffered gait disturbance.

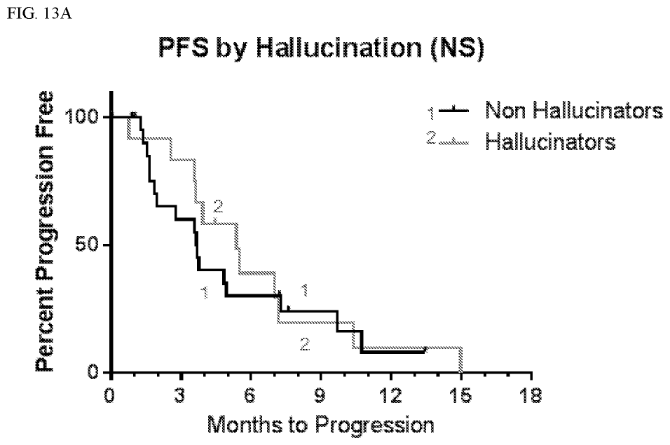

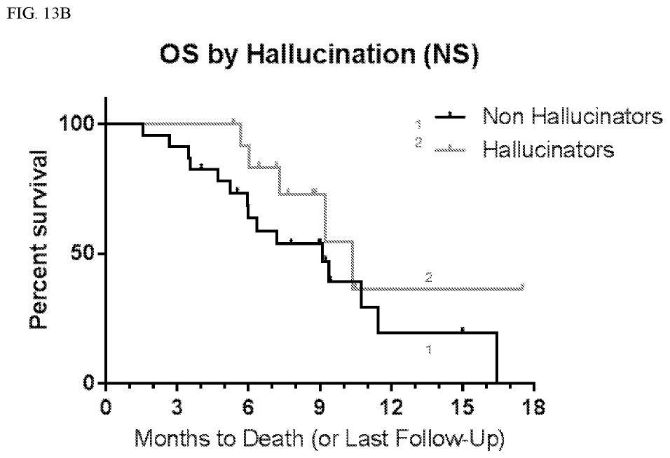

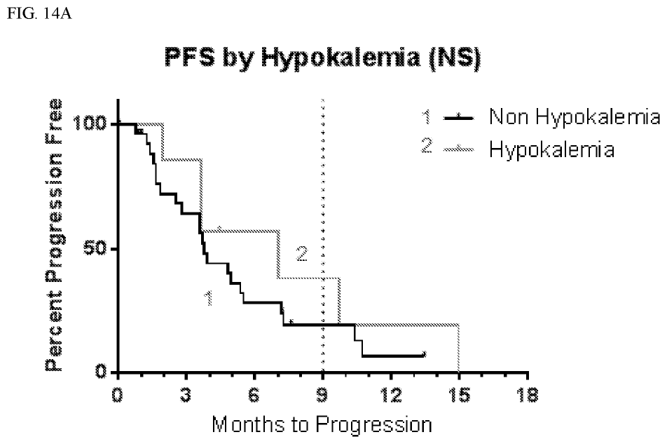

FIG. 13A shows progression free survival (PFS) according to whether patients suffered hallucinations. FIG. 13B shows overall survival (OS) according to whether patients suffered hallucinations.

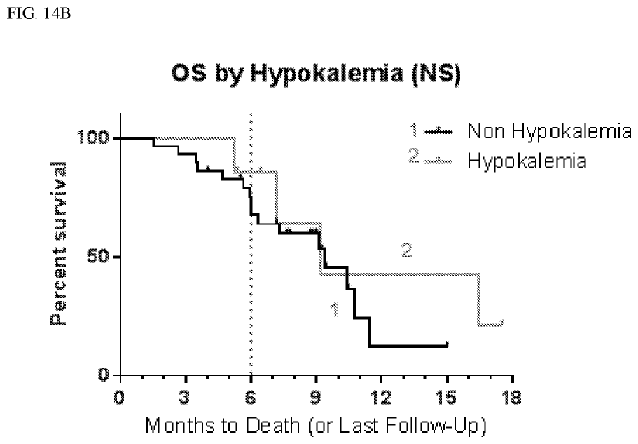

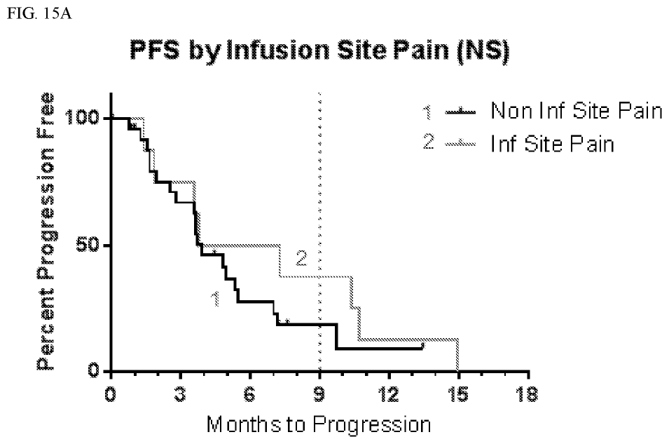

FIG. 14A shows progression free survival (PFS) according to whether patients suffered hypokalemia. FIG. 14B shows overall survival (OS) according to whether patients suffered hypokalemia.

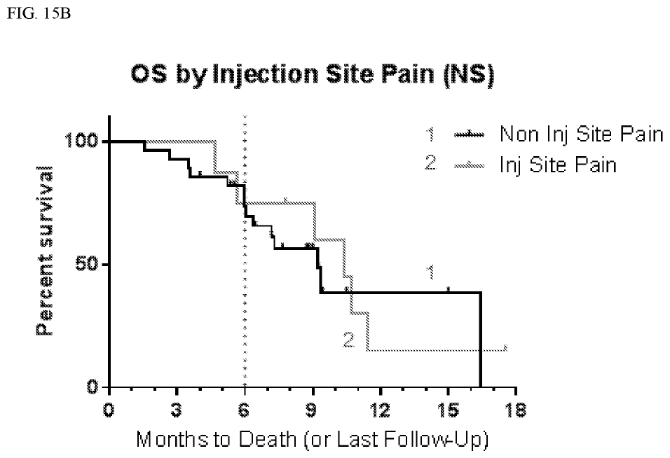

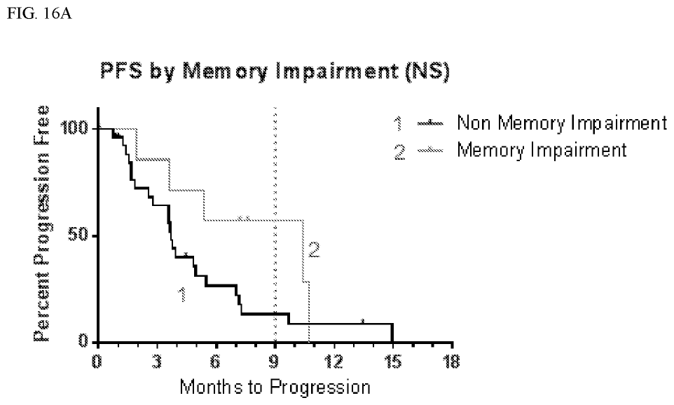

FIG. 15A shows progression free survival (PFS) according to whether patients suffered infusion site pain. FIG. 15B shows overall survival (OS) according to whether patients suffered infusion site pain.

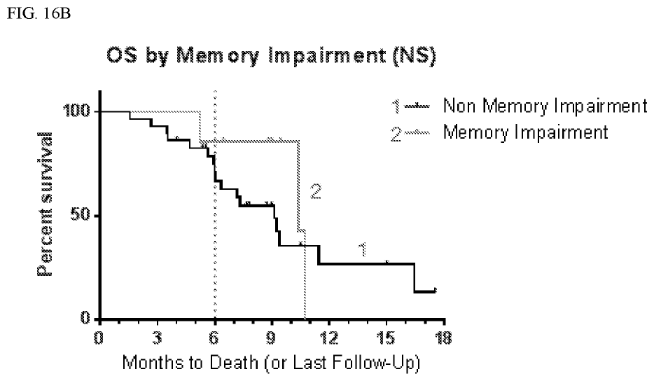

FIG. 16A shows progression free survival (PFS) according to whether patients suffered memory impairment. FIG. 16B shows overall survival (OS) according to whether patients suffered memory impairment.

Set forth in FIG. 17 is an analysis of patients as a function of whether the patients experienced hallucination. In particular, FIG. 17 shows a plot of the number of patients who did and did not experience hallucination as a function of time.

FIG. 18 shows a plot of the timing of hallucinations and dose reductions (of marizomib).

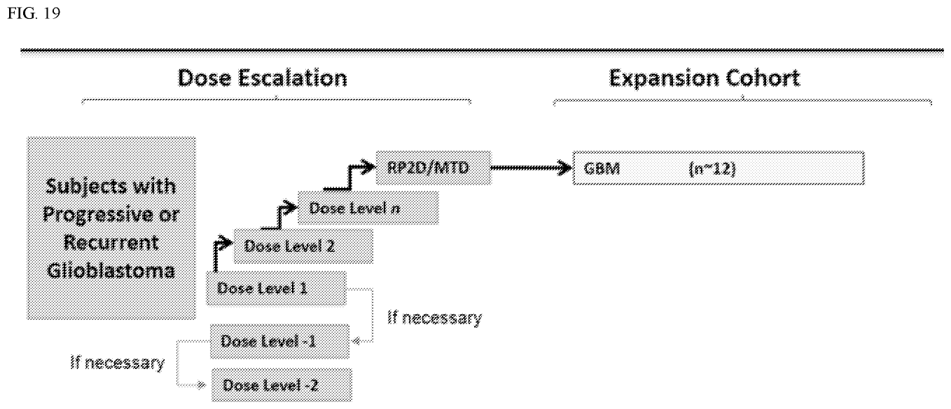

FIG. 19 is a schema of the study design set forth in Example 1. As set forth in Examples 1 and 2, standard evaluations for safety and activity are employed.

Set forth in Example 1 is a protocol for the use of marizomib in clinical trials. Without wishing to be bound by theory, the data set forth herein (e.g., in FIGS. 1-18 and Tables 1-8) was generated using the protocol set forth in Example 1. This data illustrates that an amount of the proteasome inhibitor (e.g., a therapeutic amount of marizomib) is sufficient in the context of the overall treatment regimen being provided to produce a CNS adverse event and can lead to beneficial outcomes.

### EXAMPLES

### Example 1

### Phase 1, Open-Label, Dose Escalation Study of Marizomib and Bevacizumab in WHO Grade IV Malignant Glioma

This Example gives a phase 1 dose escalation combination study followed by a Phase 2 marizomib monotherapy study.

### Study Objectives and Design

The primary objective was to determine the maximum tolerated dose and recommended phase II dose of marizomib+bevacizumab. The secondary objective was to evaluate the safety and activity of marizomib+bevacizumab.

An exploratory objective was to evaluate the baseline proteasome activity, marizomib and bevacizumab PK, marizomib neurological coordination (SARA), and quality of life assessment (FACT-Cog/FACT-Br)

### Methods

The clinical trial was a Phase 1, dose-escalation (3+3 design) followed by dose-expansion at recommended Phase 2 Dose (RP2D). Three dose escalation cohorts were used—marizomib 0.55 (6 pts), 0.7 (3 pts), and 0.8 mg/m² (3 pts); dose-expansion 0.8 mg/m² (24 pts).

Marizomib was infused intravenous (IV; 10 min) on Days 1, 8, & 15; bevacizumab was infused IV at 10 mg/kg on Days 1 and 15. The drugs were infused on 28-Day Cycles. Tumor response is assessed every other cycle by RANO criteria. Blood marizomib pharmacokinetic parameters were assessed on Day 8, serum bevacizumab pharmacokinetic parameters were assessed on days 1 and 15; blood proteasome inhibition was assessed on days 1 and 15 every cycle. Table 9 gives the treatment parameters of the present study.

TABLE 9
Treatment Parameters of Grade IV MG Study

| Cohort | IV marizomib (mg/m²) - 10 min infusion | BEV IV (mg/kg) |
|---|---|---|
| (N) | Days 1, 8, 15 q 28 days | q 14 days |
| 1 (6) | 0.55 | 10 |
| 2 (3) | 0.7 | 10 |
| 3 (3) | 0.8 | 10 |
| 4 (24) | Expansion of RP2D | 10 |
| Phase 2 | IV marizomib (0.8 mg/m²) - 10 min infusion | |
| | days 1, 8, 15, q 28 days | |
| 5 | 0.8 | None |

The key eligibility criteria included patients over 18 years of age, with histological evidence of grade IV malignant glioma in first or second relapse with clear progressive disease. Participants must have completed standard radiation therapy and temozolomide. Additional criteria included no prior proteasome inhibitor (including marizomib) or anti-angiogenic therapies, and a Karnofsky Performance Score greater than or equal to 70. Criteria also included that the patient be at least four weeks from surgical resection and 12 weeks from the end of radiotherapy. Table 10 gives the demographics of the study participants.

TABLE 10
Breakdown of Patients by Phase of Study

| | Phase 1 | Phase 2 |
|---|---|---|
| # of Subjects Enrolled (per Study Phase) | 36 | 30 |
| # of Active Subjects | 1 | 4 |
| # of Inactive Subjects | 35 | 26 |

TABLE 11
Distribution of Patients Across Sites

| | | Phase 1 | Phase 2 |
|---|---|---|---|
| Site | # of Subjects Enrolled | Active/Inactive | Active/Inactive |
| 101 | 19 | 0/15 | 0/4 |
| 102 | 21 | 0/9 | 4/8 |
| 103 | 7 | 0/3 | 0/4 |
| 104 | 7 | NA | 0/7 |
| 301 | 12 | 1/8 | 0/3 |
| Total | 66 | 1/35 | 4/26 |

TABLE 12
Demographics of Study Participants

| | MRZ + BEV | | MRZ Monotherapy | |
|---|---|---|---|---|
| | (N = 36) | | (N = 30) | |
| Age (mean ± SD years) | 55.3 ± 10.1 | | 55.8 ± 13.2 | |
| Gender | | | | |
| Male | 23 | (63.9%) | 17 | (56.7%) |
| Female | 13 | (36.1%) | 13 | (43.3%) |
| Race[1] | | | | |
| White | 30 | (83.3%) | 26 | (86.7%) |
| Black or African American | 3 | (8.3%) | 0 | |
| Asian | 3 | (8.3%) | 0 | |
| Subject Declined to Provide | 0 | | 3 | (10.0%) |
| Missing | 0 | | 1 | (3.4%) |
| Baseline KPS[2] | | | | |
| 100 | 3 | (8.6%) | 1 | (3.4%) |
| 90 | 13 | (37.1%) | 15 | (51.7%) |
| 80 | 16 | (45.7%) | 9 | (31.0%) |
| 70 | 3 | (8.6%) | 4 | (13.8%) |
| Missing | 0 | | 1 | (3.4%) |
| Time from Initial Diagnosis | 12.6 ± 7.6 | | 11.1 ± 7.4 | |
| to First Dose of Study Drug | | | | |
| (mean ± SD months) | | | | |
| Tumor Recurrence | | | | |
| First Recurrence | 20 | (55.6%) | 14 | (46.7%) |
| Second Recurrence | 7 | (19.4%) | 13 | (43.3%) |
| >2 Recurrences | 3 | (8.3%) | 0 | |
| Missing Data | 6 | (16.7%) | 3 | (10.0%) |
| Time Since Last Progressive | 0.8 ± 1.04 | | 0.5 ± 0.7 | |
| Disease to First Dose of Study | | | | |
| Drug (mean ± SD months) | | | | |
| Prior Treatment Regimens | | | | |
| Surgery | 36 | (100.0%) | 25 | (83.3%) |
| Radiation and Temozolomide | 36 | (100.0%) | 27 | (90.0%) |
| Lomustine | 4 | (11.1%) | 3 | (10.0%) |
| Investigational Drug | 1 | (2.8%) | 0 | |
| Optune Device | 0 | | 2 | (6.7%) |
| Time Since Last Radiation | 9.0 ± 6.9 | | 9.9 ± 11.1 | |
| Therapy to First Dose of Study | | | | |
| Drug (mean ± SD months) | | | | |
| Patients Receiving a | 22 | (61.1%) | 21 | (72.4%) |
| Corticosteroid at Baseline | | | | |
| (e.g., dexamethasone) | | | | |

TABLE 13
Further Demographics of Study Participants
Prior Therapies

| | | |
|---|---|---|
| Surgery, Radiation/Temozolomide | 100% | (36/36) |
| Immunotherapy | 14% | (5/36) |
| Other Investigational Drug or Device | 8% | (3/36) |
| Median months from last RT (range) | 7.8 | (2.5-29.5) |
| Corticosteroid use at baseline | 31% | (11/36) |
| Median months from last progression to C1D1 (range) | 0.8 | (0.1-3.8) |

| | |
|---|---|
| Parameter | |
| MGMT Promoter Methylation Status (6 pts unknown) | |
| Unmethylated | 20/30 |
| Methylated | 10/30 |
| EGFRvIII Positive Status (9 pts unknown) | 4/27 |
| EGFR Amplified (9 pts unknown) | 11/27 |
| EGFR Mutated (9 pts unknown) | 8/27 |

### Results

### Safety

Thirty-six patients enrolled with a median age 55 years (27-76), 64% were male, Karnofsky Score >70. Duration of dosing was 0.25-15 months to date; treatment is ongoing in 3 pts. Marizomib and bevacizumab was well tolerated.

Study treatment-related Grade ≥3 adverse events: fatigue, headache, hypertension, hallucination, confusional state, ataxia, optic nerve disorder, insomnia, delusion, hyponatremia; one Grade 4 serious adverse event (appendicitis perforated, not related to study treatment), one Grade 5 serious adverse event (embolism, intracranial hemorrhage, bevacizumab-related). One patient (cohort 1) had dose limiting toxicity (fatigue); no other dose limiting toxicities occurred across the dose range.

The efficacy evaluable population (N=33) included 31 patients efficacy evaluable by RANO criteria, and one patient Grade 5 serious adverse event (no post-treatment tumor assessment). The intent-to-treat population was 36.

One patient experienced a complete response (CR), and thirteen patients experienced partial responses (PR) (including 3 with CR for target lesion). Thirteen patients experienced stable disease (SD) (including 2 patients with unconfirmed PR), 6 patients experienced progressive disease (PD), and 3 patients were not evaluable (NE, no post-treatment tumor assessment). Marizomib and bevacizumab pharmacokinetic parameters were consistent with published parameters and not affected by co-administration. Proteasome inhibition was maximal on chymotrypsin-like (CT-L) domains in cohorts 1 and 2. Dose-dependent inhibition of trypsin-like (T-L) and caspase-like (C-L) activity in cohorts 1 vs 2 suggested dose-dependent pharmacodynamics.

TABLE 14
Most Common Treatment-Related Adverse Events

| | MRZ 0.8 mg/m² + BEV | | MRZ 0.8 mg/m² | |
|---|---|---|---|---|
| Preferred Term | Cohorts 3 & 4 (N = 27) | | Monotherapy (N = 30) | |
| Patients with at | 27 | (100.0%) | 29 | (96.7%) ^{[1]} |
| Least One TEAE | | | | |
| Fatigue | 21 | (77.8%) | 19 | (63.3%) |
| Nausea | 17 | (63.0%) | 11 | (36.7%) |
| Headache | 12 | (44.4%) | 12 | (40.0%) |
| Vomiting | 16 | (59.3%) | 9 | (30.0%) |
| Hypertension | 11 | (40.7%) | 4 | (13.3%) |
| Hallucination | 12 | (44.4%) | 12 | (40.0%) |
| Dysphonia | 10 | (37.0%) | 1 | (3.3%) |
| Confusional state | 9 | (33.3%) | 7 | (23.3%) |
| Diarrhoea | 8 | (29.6%) | 7 | (23.3%) |
| Dizziness | 8 | (29.6%) | 5 | (16.7%) |
| Epistaxis | 8 | (29.6%) | 2 | (6.7%) |
| Fall | 7 | (25.9%) | 4 | (13.3%) |
| Constipation | 7 | (25.9%) | 9 | (30.0%) |
| Anaemia | 6 | (22.2%) | 6 | (20.0%) |
| Ataxia | 7 | (25.9%) | 6 | (20.0%) |
| Hyperglycaemia | 6 | (22.2%) | 8 | (27.6%) |
| Infusion site pain | 4 | (14.8%) | 3 | (10.0%) |
| Hypokalemia | 5 | (18.5%) | 8 | (27.6%) |
| Insomnia | 3 | (11.1%) | 14 | (46.7%) |
| Platelet count | 5 | (18.5%) | 7 | (23.3%) |
| decreased | | | | |
| Dysarthria | 6 | (22.2) | 5 | (16.7%) |

TABLE 15
Most Common Treatment-Related Adverse Events By Cause

| | MRZ 0.8 mg/m² + BEV | | | | | | MRZ 0.8 mg/m² | |
|---|---|---|---|---|---|---|---|---|
| Preferred | Cohorts 3 & 4 (N = 27) | | | | | | Monotherapy (N = 30) ^{[1]} | |
| Term | MRZ Related | | BEV Related | | Related to Both | | MRZ Related | |
| Fatigue | 19 | (70.4%) | 18 | (66.7%) | 18 | (66.7%) | 18 | (60.0%) |
| Headache | 12 | (44.4%) | 6 | (22.2%) | 6 | (22.2%) | 11 | (36.7%) |
| Nausea | 17 | (63.0%) | 2 | (7.4%) | 2 | (7.4%) | 9 | (30.0%) |
| Vomiting | 15 | (55.6%) | 4 | (14.8%) | 4 | (14.8%) | 7 | (23.3) |
| Hypertension | 1 | (3.7%) | 11 | (40.7%) | 1 | (3.7%) | 1 | 93.3%) |
| Hallucination | 12 | (44.4%) | 0 | (0.0%) | 0 | (0.0%) | 11 | (36.7%) |
| Dysphonia | 1 | (3.7%) | 10 | (37.0%) | 1 | (3.7%) | 0 | |
| Diarrhoea | 8 | (29.6%) | 1 | (3.7%) | 1 | (3.7%) | 7 | (23.3) |
| Dizziness | 8 | (29.6%) | 1 | (3.7%) | 1 | (3.7%) | 3 | (10.0%) |
| Infusion site pain | 4 | (14.8%) | 1 | (3.7%) | 1 | (3.7%) | 2 | (6.7%) |
| Confusional state | 7 | (25.9%) | 1 | (3.7%) | 1 | (3.7%) | 6 | (20.0%) |
| Epistaxis | 0 | (0.0%) | 7 | (25.9%) | 0 | (0.0%) | 1 | (3.3%) |
| Ataxia | 7 | (25.9%) | 0 | (0.0%) | 0 | (0.0%) | 5 | (16.7%) |
| Anaemia | 5 | (18.5%) | 0 | (0.0%) | 0 | (0.0%) | 5 | (16.7% |
| Constipation | 5 | (18.5%) | 1 | (3.7%) | 1 | (3.7%) | 6 | (20.0%) |
| Platelet count decreased | 2 | (7.4%) | 3 | (11.1%) | 1 | (3.7%) | 0 | |
| Muscular weakness | 3 | (11.1%) | 1 | (3.7%) | 1 | (3.7%) | 0 | |
| Stomatitis | 3 | (11.1%) | 2 | (7.4%) | 2 | (7.4%) | 0 | |
| Upper-airway | 0 | (0.0%) | 5 | (18.5%) | 0 | (0.0%) | 0 | |
| cough syndrome | | | | | | | | |
| Fall | 3 | (11.1%) | 0 | (0.0%) | 0 | (0.0%) | 1 | (3.3%) |

TABLE 16
Treatment Related Grade ≥3 AEs by Patient

| | MRZ Dose | | | | | | |
|---|---|---|---|---|---|---|---|
| | 0.55 mg/m² | | 0.7 mg/m² | | 0.8 mg/m² | | |
| | Cohort 1 | | Cohort 2 | | Cohorts 3 & 4 | | |
| | (N = 6) | | (N = 3) | | (N = 27) | | TOTAL |
| Preferred Term | BEV | MRZ | BEV | MRZ | BEV | MRZ | (N = 36) |
| Ataxia | 0 | 0 | 0 | 0 | 0 | 1 | 1 |
| Confusional | 0 | 1 | 0 | 0 | 0 | 0 | 1 |
| State | | | | | | | |
| Delusion | 0 | 0 | 0 | 0 | 0 | 1 | 1 |
| Fatigue | 1 | 1 | 0 | 0 | 1 | 1 | 2 |
| Hallucination | 0 | 1 | 0 | 0 | 0 | 1 | 2 |
| Headache | 1 | 0 | 0 | 0 | 1 | 3 | 4 |
| Insomnia | 0 | 0 | 0 | 0 | 0 | 1 | 1 |
| Embolism | 0 | 0 | 0 | 0 | 1 | 0 | 1 |
| Hypertension | 0 | 0 | 1 | 0 | 5 | 0 | 6 |
| Intracranial | 0 | 0 | 0 | 0 | 1 | 0 | 1 |
| Hemorrhage | | | | | | | |
| Embolism | 0 | 0 | 0 | 0 | 1 | 0 | 1 |
| Optic Nerve | 0 | 0 | 0 | 0 | 1 | 0 | 1 |
| Disorder | | | | | | | |
| Proteinuria | 0 | 0 | 1 | 0 | 0 | 0 | 1 |
| Fall | 0 | 0 | 0 | 0 | 0 | 1 | 1 |
| Dyspnea | 0 | 0 | 0 | 0 | 1 | 1 | 1 |
| Hyponatremia | 0 | 0 | 0 | 0 | 0 | 1 | 1 |

Table 17 gives the study treatment-related adverse events and all adverse events greater than or equal to grade 3, as of 12 September 2016.

TABLE 17
Treatment-Related AEs and All AEs ≥3

| Study Treatment-Related | | | | | | | |
|---|---|---|---|---|---|---|---|
| Adverse Events and All | | | | | | | |
| Grade 3 or Above Adverse | # Patients (%) | | Relationship to Study Treatment | | | | # Patients |
| EventsPreferred Term | with AE | | Neither | BEV | MRZ | Both | Grade ≥3 |
| Fatigue | 24 | (67) | 2 | 0 | 1 | 21 | 3 |
| Nausea | 21 | (58) | 0 | 0 | 19 | 2 | 0 |
| Headache | 20 | (56) | 2 | 1 | 5 | 12 | 5 |
| Vomiting | 17 | (47) | 1 | 0 | 12 | 4 | 0 |
| Hypertension | 16 | (42) | 1 | 13 | 0 | 2 | 6 |
| Hallucination | 11 | (31) | 0 | 0 | 11 | 0 | 2 |
| Diarrhoea | 10 | (28) | 0 | 0 | 9 | 1 | 0 |
| Dysphonia | 10 | (28) | 0 | 10 | 0 | 0 | 0 |
| Dizziness | 9 | (25) | 0 | 0 | 8 | 1 | 0 |
| Anaemia | 8 | (22) | 2 | 0 | 6 | 0 | 0 |
| Confusional | 8 | (22) | 1 | 0 | 6 | 1 | 1 |
| State | | | | | | | |
| Epistaxis | 8 | (22) | 1 | 7 | 0 | 0 | 0 |
| Hyperglycemia | 8 | (22) | 8 | 0 | 0 | 0 | 2 |
| Falls | 8 | (22) | 5 | 0 | 3 | 0 | 0 |
| Hypokalemia | 7 | (19) | 7 | 0 | 0 | 0 | 1 |
| Constipation | 7 | (19) | 2 | 0 | 4 | 1 | 0 |
| Ataxia | 7 | (19) | 1 | 0 | 6 | 0 | 1 |
| Convulsion | 7 | (19) | 7 | 0 | 0 | 0 | 0 |
| Dysarthria | 7 | (19) | 6 | 0 | 1 | 0 | 1 |
| Muscular | 6 | (17) | 4 | 0 | 2 | 0 | 2 |
| Weakness | | | | | | | |
| Infusion Site | 6 | (17) | 0 | 0 | 6 | 0 | 0 |
| Pain | | | | | | | |
| Anxiety | 6 | (17) | 6 | 0 | 0 | 0 | 0 |
| Vision Blurred | 6 | (17) | 3 | 0 | 3 | 0 | 0 |
| Hemiparesis | 5 | (14) | 5 | 0 | 0 | 0 | 3 |
| Insomnia | 4 | (11) | 2 | 1 | 1 | 0 | 1 |
| Dysphagia | 3 | (8) | 3 | 0 | 0 | 0 | 1 |
| Hypotension | 3 | (8) | 3 | 0 | 0 | 0 | 1 |
| Lymphocyte | 3 | (8) | 3 | 0 | 0 | 0 | 3 |
| Count | | | | | | | |
| Decreased | | | | | | | |
| Dyspnoea | 3 | (8) | 2 | 0 | 0 | 1 | 1 |
| Pyramidal Tract | 3 | (8) | 3 | 0 | 0 | 0 | 1 |
| Syndrome | | | | | | | |
| Haemorrhage | 2 | (6) | 0 | 2 | 0 | 0 | 1 (Grade 5) |
| Intracranial | | | | | | | |
| Aphasia | 2 | (6) | 2 | 0 | 0 | 0 | 1 |
| Asthenia | 2 | (6) | 2 | 0 | 0 | 0 | 1 |
| Embolism | 2 | (6) | 0 | 2 | 0 | 0 | 1 |
| Hyponatremia | 2 | (6) | 1 | 0 | 1 | 0 | 1 |
| Fracture of | 1 | (3) | 1 | 0 | 0 | 0 | 1 |
| Femur | | | | | | | |
| Tumor | 1 | (3) | 1 | 0 | 0 | 0 | 1 |
| Metastasis | | | | | | | |
| Optic Nerve | 1 | (3) | 0 | 1 | 0 | 0 | 1 |
| Disorder | | | | | | | |
| Depressed | 1 | (3) | 1 | 0 | 0 | 0 | 1 |
| Level of | | | | | | | |
| Consciousness | | | | | | | |
| Delusion | 1 | (3) | 0 | 0 | 1 | 0 | 1 |
| Appendicitis | 1 | (3) | 1 | 0 | 0 | 0 | 1 (Grade 4) |
| Perforated | | | | | | | |
| Ear Infection | 1 | (3) | 1 | 0 | 0 | 0 | 1 |

As shown above, the combination of marizomib and bevacizumab is generally well-tolerated in patients with recurrent glioma. The most common marizomib-related adverse events include fatigue, headache, nausea, vomiting, and hallucinations. The most common marizomib-related adverse events ≥3 were headache (3) and confusional state (3). Four patients experienced marizomib-related serious adverse events (hallucinations/confusion; confusion/fatigue/muscle weakness; confusion; and cough/dyspnea). Three grade-4 adverse events were observed: blindness (bevacizumab-related); appendicitis perforated (not related); depressed level of consciousness (not related). Three grade-5 adverse events were observed: intracranial hemorrhage (BEV-related); and disease progression (in two patients; not related). One patient experienced dose-limiting toxicity: in cohort 1, because of fatigue (MRZ-related; not a serious adverse event).

### Efficacy

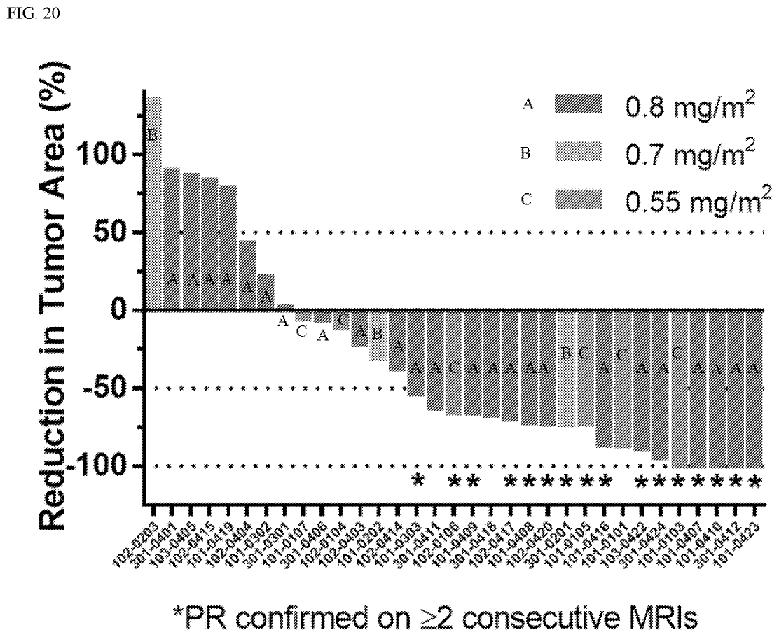

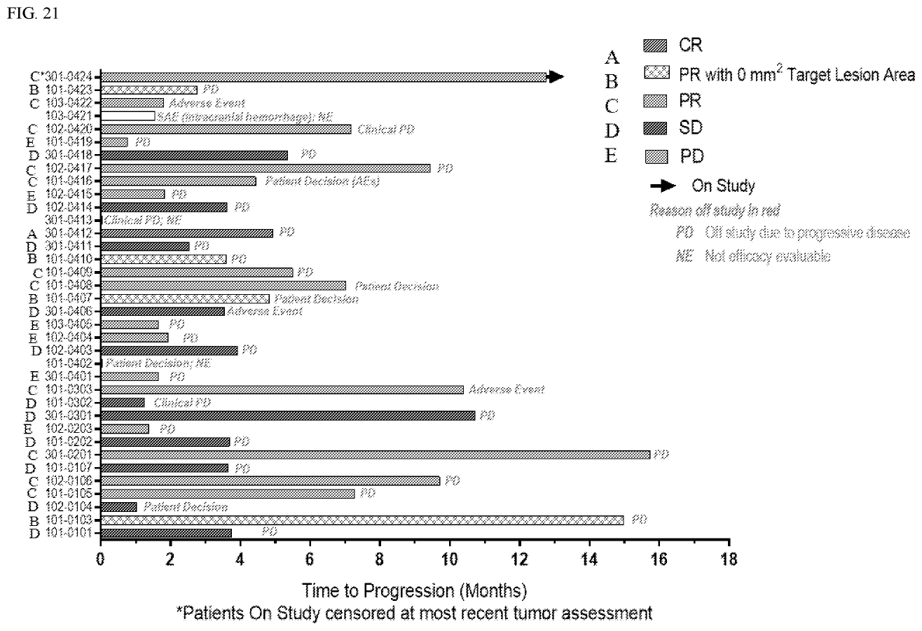

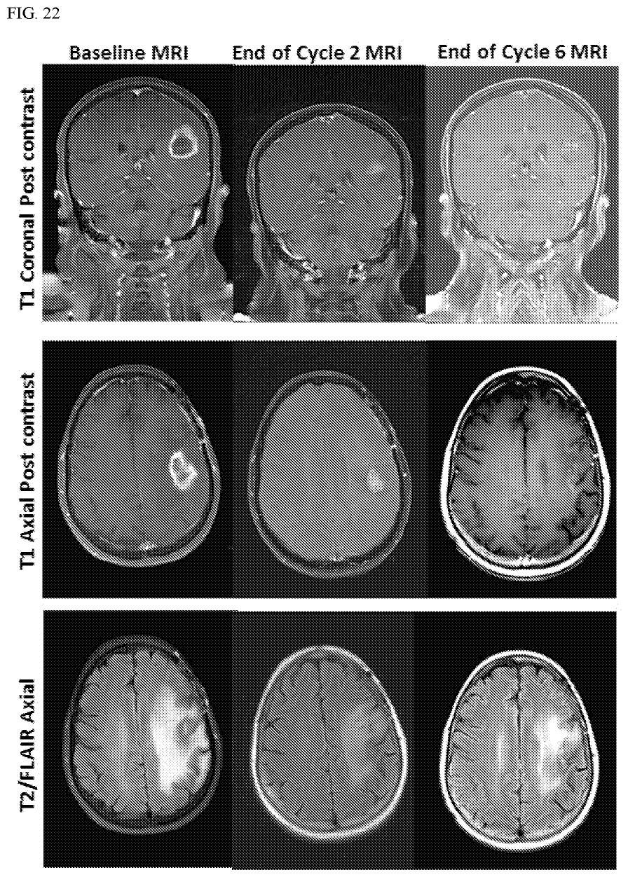

FIG. 20 shows a plot of the best responses by RANO criteria for the 33 patients. FIG. 20 demonstrates that 25 of the 33 efficacy evaluable patients achieved a clinical benefit (RANO≥Stable Disease) from marizomib and bevacizumab treatment. FIG. 21 shows the time to progression in the patients in the present clinical trial. Table 18 likewise shows the response rate by RANO. Table 19 shows the response rate by MGMT Promoter methylation status.

TABLE 18
Response Rate by RANO

| | Number | % Efficacy | % Intent |
|---|---|---|---|
| | of | Evaluable | to Treat |
| Best Response by RANO | responses | (N = 33) | (N = 36) |
| CR (1) + CR target/PR overall | 16 | 48% | 44% |
| (4) + PR (11) | | | |
| SD (including 2 unconfirmed PR) | 11 | 33% | 31% |
| PD | 6 | 18% | 17% |
| NE | 3 | NA | 8% |

TABLE 19
Response Rate by MGMT Promoter Methylation Status

| | Evaluable (N = 33) Efficacy | | | ITT (N = 36) | | |
|---|---|---|---|---|---|---|
| Best Response | Unmethylated* | Methylated | Unknown | Unmethylated* | Methylated | Unknown |
| (N) | N = 19 | N = 8 | N = 6 | N = 20 | N = 10 | N = 6 |
| CR/PR (14) | 7 | 5 | 2 | 7 | 5 | 2 |
| SD (13) | 9 | 1 | 3 | 9 | 1 | 3 |
| PD (6) | 3 | 2 | 1 | 3 | 2 | 1 |
| NE (3) | — | — | — | 1 | 2 | 0 |

*Unmethylated: <8% promoter methylation by pyro-sequencing

The overall response rate was 42% (RANO≥partial response) for the Efficacy Evaluable (EE) and 39% in the Intent To Treat (ITT) population. Five of the fourteen partial responses were complete responses for target tumor area (0 mm²) on greater than or equal to 2 consecutive MRIs.

### Examples of Target Lesion Complete Response

A 59-year old female patient (Patient A) had a Karnofsky performance score of 90 prior to treatment with marizomib and bevacizumab. Patient A had a brain tumor resection in October 2014. Between December 2014 and January 2015 Patient A was treated with radiotherapy and temozolomide. Between February 2015 and April 2015, Patient A received three cycles of temozolomide. In early April 2015, progressive disease (PD) was confirmed.

Patient A started marizomib treatment (0.55 mg/m²) plus bevacizumab in late May 2015. After 2 cycles, the patient had a dose reduction to 0.4 mg/m² C3D1.

FIG. 22 shows nine MRI images of Patient A, who achieved a complete response after treatment with marizomib and bevacizumab. The first column shows baseline MRI images, the middle column shows images after cycle 2, and the third column shows images after the end of cycle 6. The top row shows the T1 coronal post contrast, the middle row shows the T1 axial post contrast, and the bottom row shows the T2/FLAIR axial images.

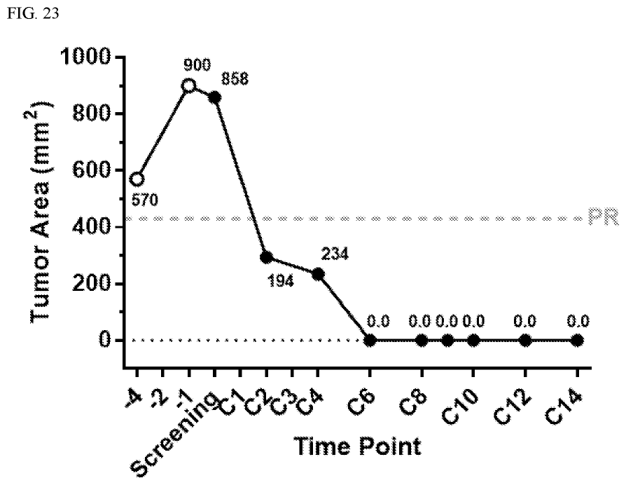

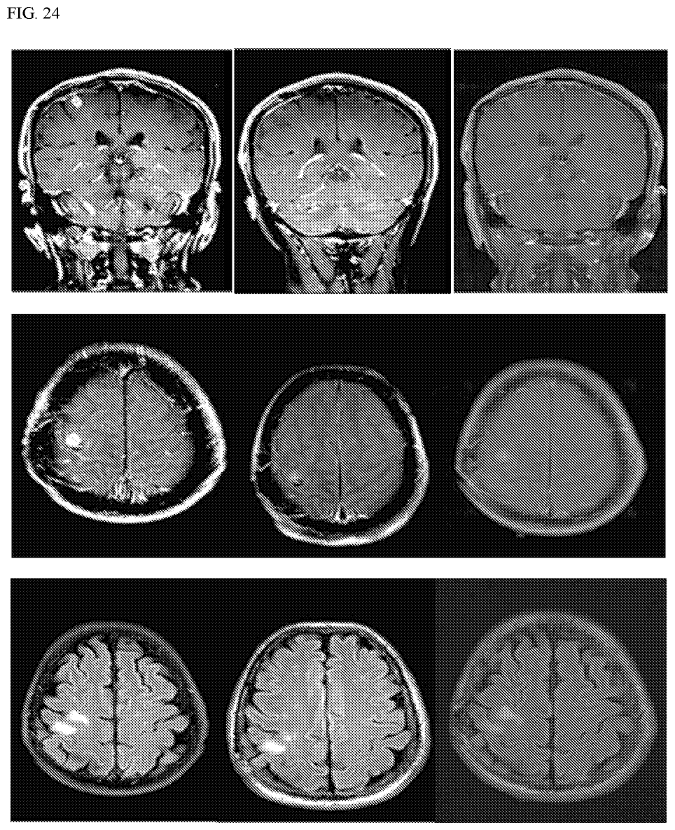

FIG. 23 shows a plot of Patient A′s tumor size as a function of time and the number of cycles Patient A received. As shown in FIG. 23, the tumor area was reduced to 0 mm² by the sixth cycle of treatment.

A 54-year old male patient (Patient B) had a Karnofsky performance score of 90 prior to treatment with marizomib and bevacizumab. Patient B had a brain tumor resection in October 2014. Between November 2014 and January 2015, Patient B was treated with radiotherapy and temozolomide. Between February 2015 and June 2015, Patient B received five cycles of temozolomide. In late June 2015, Progressive Disease (PD) was confirmed.

Patient B started marizomib treatment (0.55 mg/m²) plus bevacizumab in late July 2015. Patient B was removed from the study in March 2016 due to PD.

FIG. 24 shows MRI images of Patient B. The first column shows baseline MRI images, the middle column shows images after cycle 2, and the third column shows images after the end of cycle 4. The top row shows the T1 coronal post contrast, the middle row shows the T1 axial post contrast, and the bottom row shows the T2/FLAIR axial images.

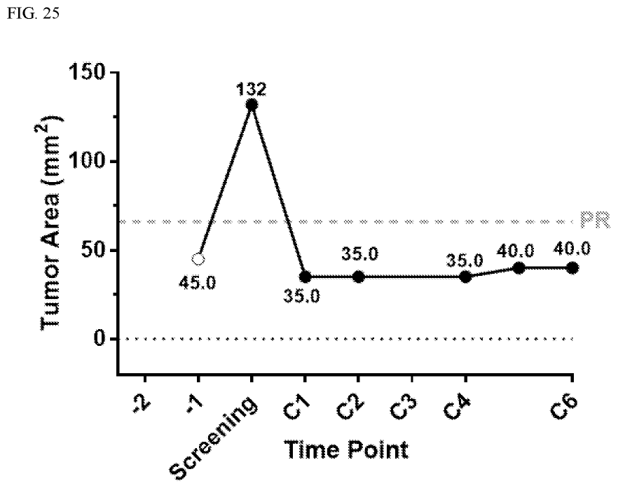

FIG. 25 shows a plot of Patient B's tumor size as a function of time and the number of cycles Patient B received. As shown in FIG. 25, the tumor area was reduced to 0 mm² by the fourth cycle of treatment.

A 61-year old male patient (Patient C) had a Karnofsky performance score of 80 prior to treatment with marizomib and bevacizumab. Patient C had a brain tumor resection in March 2015. Between April 2015 and May 2015 Patient C was treated with radiotherapy and temozolomide. Between June 2015 and July 2015, Patient C received two cycles of temozolomide. In August 2015, progressive disease (PD) was confirmed.

Patient C started marizomib treatment (0.55 mg/m²) plus bevacizumab in August 2015.

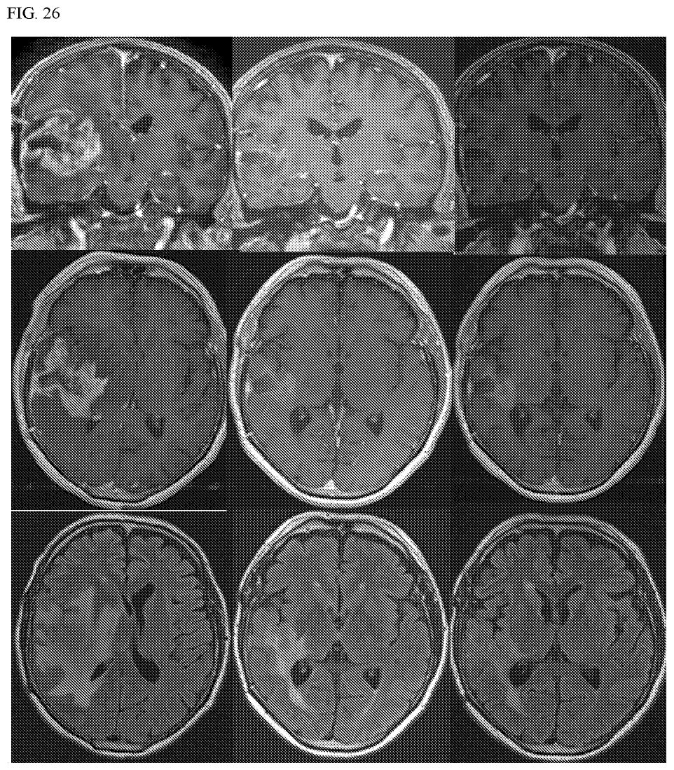

FIG. 26 shows MRI images of Patient C. The first column shows baseline MRI images, the middle column shows images after cycle 2, and the third column shows images after the end of cycle 4. The top row shows the T1 coronal post contrast, the middle row shows the T1 axial post contrast, and the bottom row shows the T2/FLAIR axial images.

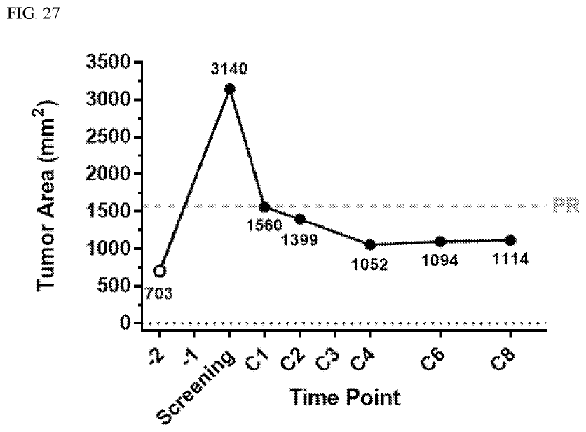

FIG. 27 shows a plot of Patient C's tumor size as a function of time and the number of cycles Patient C received. As shown in FIG. 27, the tumor area was reduced to about a third of its peak volume after four cycles of treatment.

A 53-year old male patient (Patient D) had a Karnofsky performance score of 90 prior to treatment with marizomib and bevacizumab. Patient D had a brain tumor resection in April 2015. Between April 2015 and June 2015 Patient D was treated with radiotherapy and temozolomide. Between July 2015 and August 2015, Patient D received three cycles of temozolomide. In September 2015, progressive disease (PD) was confirmed.

Patient D started marizomib treatment (0.7 mg/m²) plus bevacizumab in late September 2015.

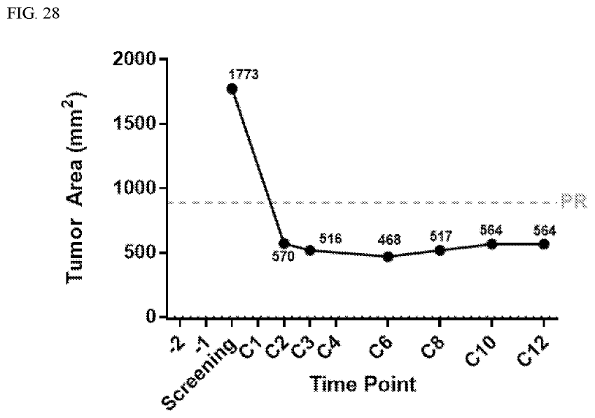

FIG. 28 shows a plot of Patient D's tumor size as a function of time and the number of cycles Patient D received. As shown in FIG. 28, the tumor area was reduced to about a third of its peak volume after two cycles of treatment.

A 64-year old male patient (Patient E) had a Karnofsky performance score of 90 prior to treatment with marizomib and bevacizumab. Patient E had a brain tumor resection in October 2014. Between November 2014 and December 2014 Patient E was treated with radiotherapy and temozolomide. Between February 2015 and September 2015, Patient E received temozolomide, and from February 2015 to October 2015 Patient E also received Novocure TTF treatment. In October 2015, PD was confirmed.

Patient E started marizomib treatment (0.8 mg/m²) plus bevacizumab in early February 2016.

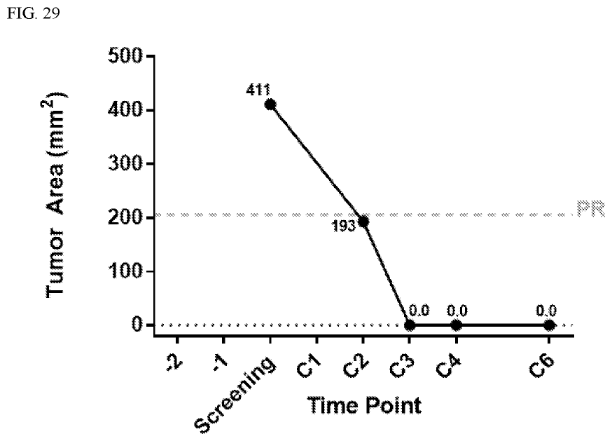

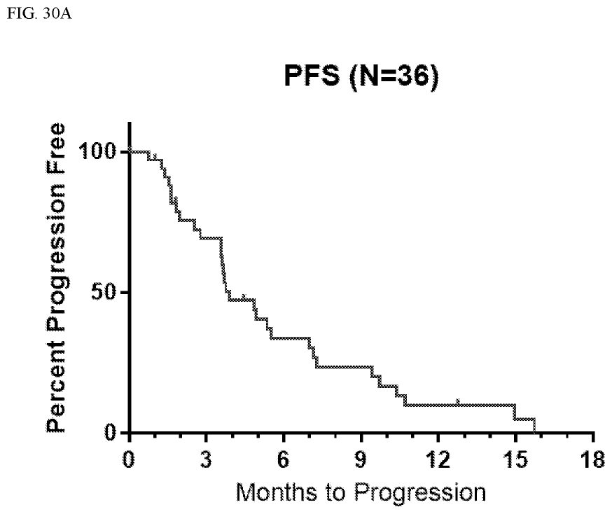

FIG. 29 shows a plot of Patient E's tumor size as a function of time and the number of cycles Patient E received. As shown in FIG. 29, the tumor area was reduced to about 0 mm² after three cycles of treatment.

### Progression Free Survival (PFS): Overall and by MGMT Promoter Methylation Status

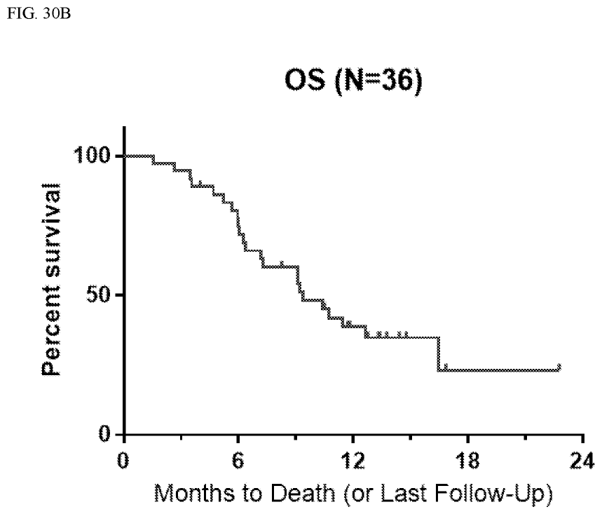

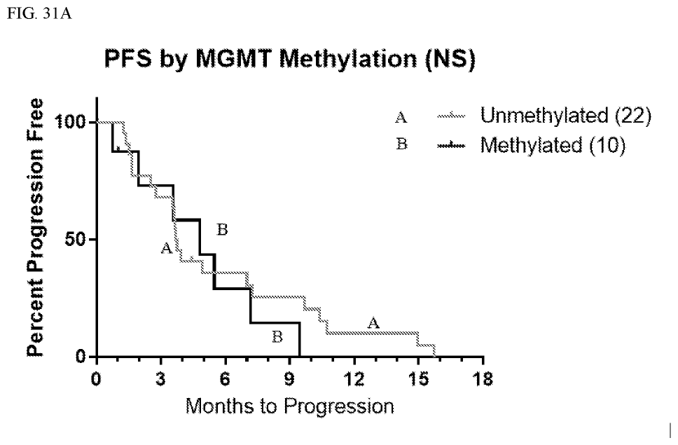

FIG. 30A shows a plot of the progression free survival (PFS) percent as a function of time for all patients. FIG. 30B shows a plot of the overall survival (OS) percent as a function of time for all patients.

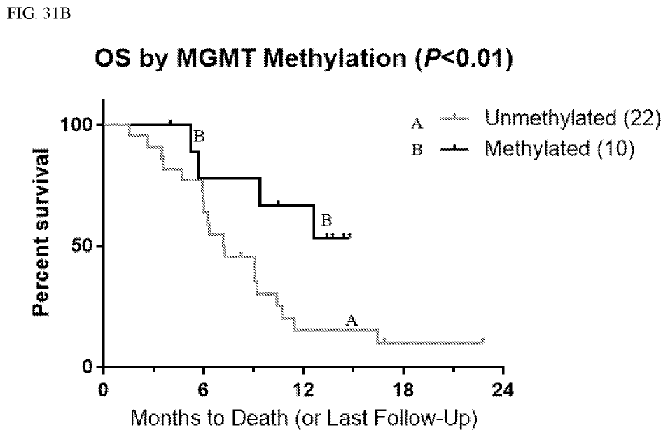

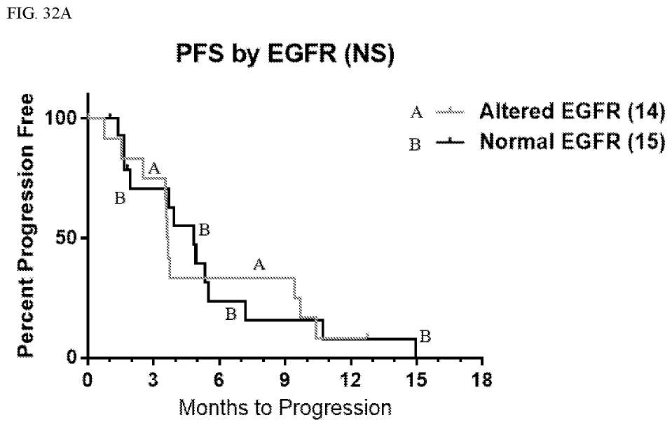

FIG. 31A shows a plot of the PFS percent as a function of time for patients by O 6-methylguanine-DNA methyltransferase (MGMT) promoter methylation status (methylated or unmethylated). FIG. 31B shows a plot of overall survival (OS) percent as a function of time for patients by O 6-methylguanine-DNA methyltransferase (MGMT) promoter methylation status (methylated or unmethylated).

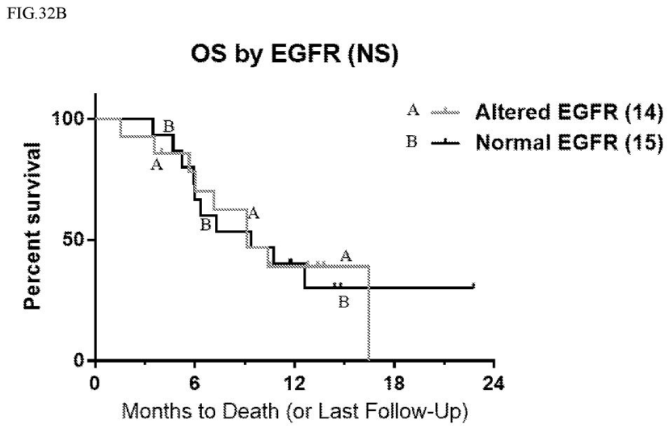

FIG. 32A shows progression free survival (PFS) as a function of time for patients by EGFR status. FIG. 32B shows overall survival (OS) as a function of time for patients by EGFR status.

Without wishing to be bound by theory, the percentage of patients treated with marizomib and bevacizumab who have not progressed at six months was higher than patients treated with bevacizumab only. The percentage of patients with six months PFS treated with marizomib and bevacizumab was about twice that among all patients, and about four times that in patients with unmethylated MGMT promoter, in comparison with patients treated with bevacizumab only. Without wishing to be bound by theory, unmethylated MGMT promoter is a biomarker of poor prognosis in malignant glioma. Patients with unmethylated MGMT promoter can be more likely to suffer recurrent disease, and for recurrence to occur more quickly than in patients with methylated MGMT promoter. For instance, patients with unmethylated MGMT promoter who are treated with the standard of care (temozolomide and radiotherapy) can be more likely to relapse.

TABLE 20
MRZ + BEV RANO Resnonse Rate by MGMT Promoter Methylation Status

| | Efficacy Evaluable (N = 33) | | | Intent to Treat (N = 36) | | |
|---|---|---|---|---|---|---|
| Best Response | Unmethylated | Methylated | Unknown | Unmethylated | Methylated | Unknown |
| (N) | (N = 21) | (N = 8) | (N = 6) | (N = 22) | (N = 10) | (N = 4) |
| CR or PR (16) | 9 | 5 | 2 | 9 | 5 | 2 |
| SD (11) | 9 | 1 | 1 | 9 | 1 | 1 |
| PD (6) | 3 | 2 | 1 | 3 | 2 | 1 |
| NE (3) | — | — | — | 1 | 2 | 0 |

TABLE 21
MRZ + BEV PFS by MGMT Promoter Methylation Status

| | PFS | | | | | |
|---|---|---|---|---|---|---|
| | No. | Median | 6 Months | 9 Months | 12 Months | 18 Months |
| | Censored | (Months) | % | % | % | % |
| Unmethylated (22) | 1 | 3.7 | 36 | 26 | 10 | 0 |
| Methylated (10) | 3 | 4.8 | 29 | 15 | 0 | 0 |

TABLE 22
MRZ + BEV OS by MGMT Promoter Methylation Status

| | OS | | | | | |
|---|---|---|---|---|---|---|
| | No. | Median | 6 Months | 9 Months | 12 Months | 18 Months |
| | Censored | (Months) | % | % | % | % |
| Unmethylated (22) | 3 | 7.2 | 68 | 45 | 15 | 10 |
| Methylated (10) | 6 | Undefined | 78 | 78 | 47 | 53 |

As shown in the tables above, patients with unmethylated MGMT promoter had similar rates of overall survival and progression free survival as patients with methylated MGMT promoter.

### Marizomib Monotherapy

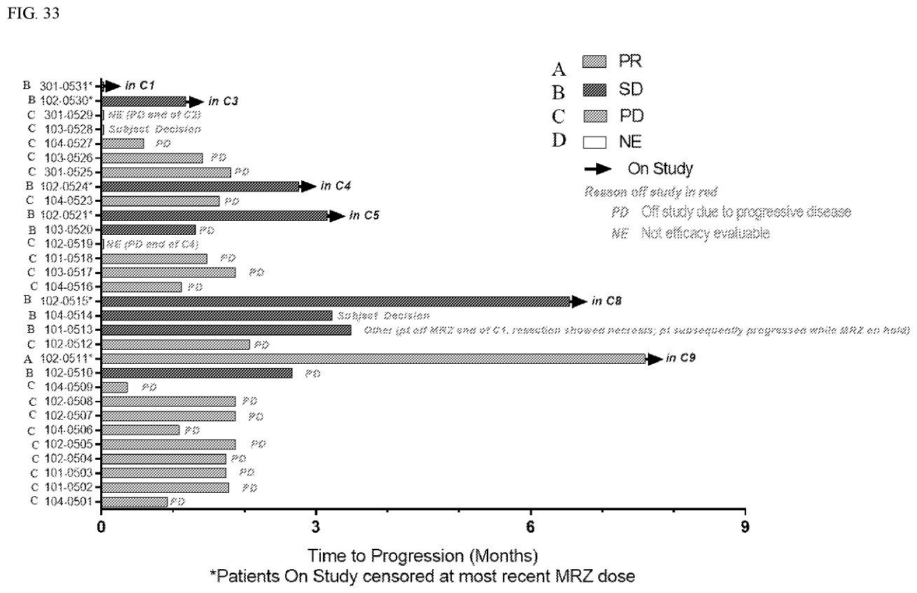

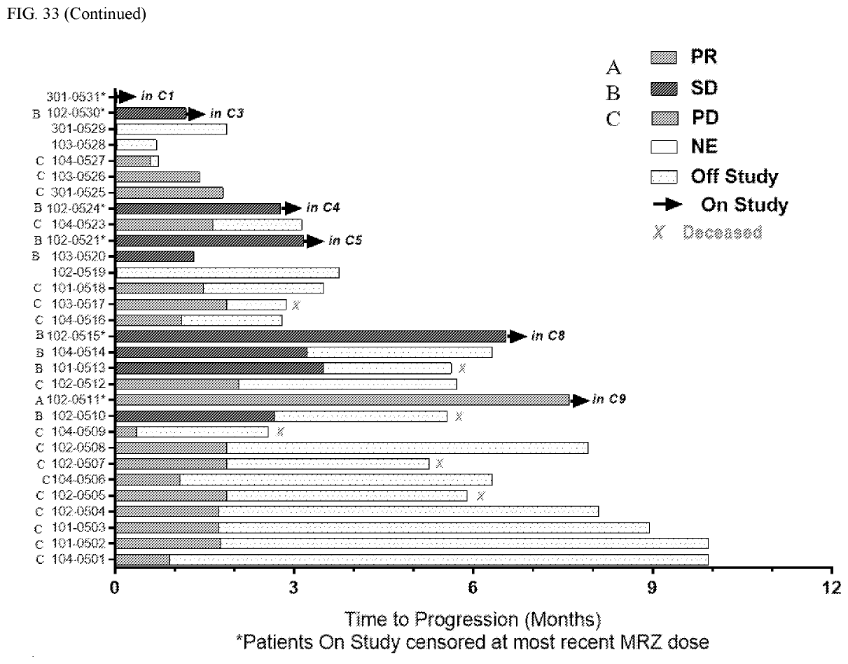

FIG. 33 shows a time to progression for patients undergoing monotherapy with marizomib.

TABLE 23
Response of Patients Treated with Marizomib Monotherapy

| | ITT (N = 30) | | |
|---|---|---|---|
| Best Response | Unmethylated* | Methylated | Unknown |
| (N) | N = 18 | N = 7 | N = 5** |
| CR (0) | 0 | 0 | 0 |
| PR (1) | 1 | 0 | 0 |
| SD (8) | 4 | 3 | 1 |
| PD (19) | 12 | 4 | 3 |
| NE (2) | 1 | 0 | 1 |

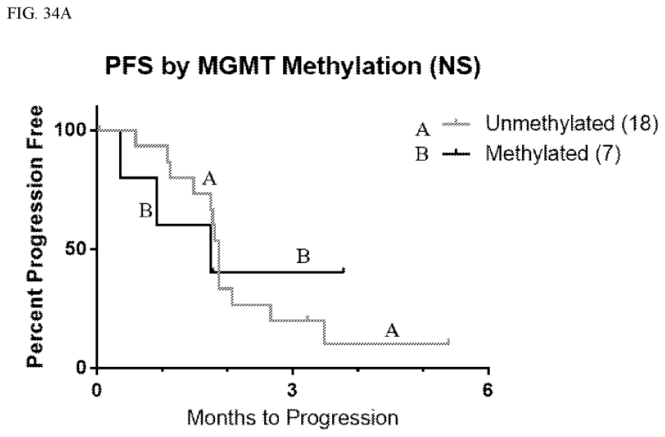

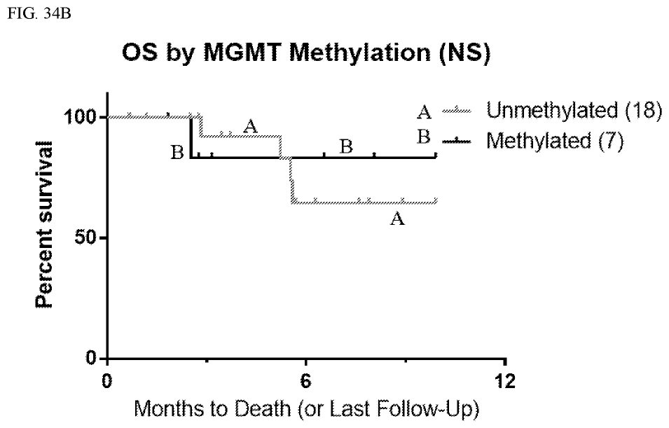

FIG. 34A shows a plot of progression-free survival for patients treated with marizomib monotherapy by methylation status. FIG. 34B shows a plot of overall survival for patients treated with marizomib monotherapy by methylation status.

TABLE 24
Progression-Free Survival and Overall Survival
of Patients treated with Marizomib Monotherapy

| | PFS | | | | OS | | | |
|---|---|---|---|---|---|---|---|---|
| | # | Median | 3 mo | 6 mo | # | Median | 6 mo | 9 mo |
| | Censored | (months) | % | % | Censored | (months) | % | % |
| Unmethylated (18) | 5 | 1.9 | 20 | NA | 14 | Undefined | 65 | 65 |
| Methylated (7) | 4 | 1.7 | 40 | NA | 6 | Undefined | 83 | 83 |

TABLE 25
Progression Free Survival (PFS): Overall and by EGFR Status

| | MGMT | | | |
|---|---|---|---|---|
| Pt ID | Promoter | EGFR* | IDH1 | TP53 |
| 0511 | Unmethylated | Normal | WT | Pathogenic Mutation |
| 0513 | Unmethylated | Unknown | Unknown | Unknown |
| 0514 | Unmethylated | Normal | WT | WT |
| 0515 | Methylated | Normal | WT | WT |
| 0521 | Methylated | Normal | R132H | Pathogenic Mutation |
| 0524 | Methylated | Normal | R132H | Pathogenic Mutation |

*Normal EGFR status = Not amplified or mutated, EGFRVIII negative

As set forth above, methylation of the MGMT promoter was determined for 27 of 30 patients. Eighteen of 27 had unmethylated MGMT promoter (67%). Additionally, 9/25 patients had altered EGFR (36%). Of these, 8 had amplified EGFR, 7 had mutated EGFR (6 of 7 also amplified) and 2 were EGFRVIII positive (both were also EGFR amplified). Four of 25 patients had the IDH1 mutation R132H (16%), and 7/25 patients had pathogenic TP53 mutations (28%). Without wishing to be bound by theory, the IDH1 mutation is commonly associated with a lower grade tumor which can subsequently progress to a grade IV malignant glioma (e.g., glioblastoma, GBM). Without wishing to be bound by theory, TP53 is a tumor suppressor. Pathogenic mutations in TP53 can suppress its activity, and in some embodiments lead to a more aggressive tumor type.

TABLE 26
MRZ Monotherapy: 4 Patients ≥ Static Disease and >4 Cycles

| Pt | Screening | | Recur- | Best | | Current |
|---|---|---|---|---|---|---|
| ID | SPD* | Diagnosis | rence | Response | SPD | Cycle |
| 0511 | 703.47 | 05/15 | 2nd | PR | 231.3 | 10 |
| 0515 | 199.6 | 06/15 | 1st | SD | 259.5 | 9 |
| 0521 | 836.44 | 02/16 | 1^{st} | SD | 832.82 | 5 |
| 0524 | 122.55 | 07/15 | 1^{st} | SD | NA | 5 |
| | | (07/14 | | | | |
| | | astrocytoma) | | | | |

As set forth in Table 26, four patients were found to respond to marizomib monotherapy.

In summary, most patients demonstrated rapid progression when treated with marizomib alone. Six patients were on the study for >4 cycles (Table 25). All were of normal EGFR status. Three of the six patients had pathogenic TP53 mutations. Comparable tumor responses and PFS/OS results in unmethylated compared with methylated MGMT promoter status was observed.

### Bevacizumab Monotherapy

Table 27 shows a comparison of the present study with a clinical trial evaluating single-agent bevacizumab in recurrent glioma for comparison.

TABLE 27
Single Agent Bevacizumab Comparator Data in Recurrent Glioma

6 Months PFS

| Study | All | uMGMT Promoter |
|---|---|---|
| Present Study | 34% | 34% |
| BELOB Trial (BEV monotherapy) | 16% | 8% |
| (Taal et al., 2014) | | |

### Comparison Marizomib Monotherapy and Marizomib-Bevacizumab Combination

TABLE 28
MRZ + BEV Improved PFS & OS at 6, 9 and 12 Months Compared with Historical BEV Monotherapy Studies

| | | 6 mo Progression Free Survival | | | 9 mo Progression Free Survival | | | 12 Mo. Progression Free Survival | | |
|---|---|---|---|---|---|---|---|---|---|---|
| | | All | Un- | | All | Un- | | All | Un- | |
| Study | Treatment | Pts | methylated | Methylated | Pts | methylated | Methylated | Pts | methylated | Methylated |
| Current | MRZ + BEV | 34% | 36% | 29% | 24% | 26% | 15% | 10% | 10% | 0% |
| Study | | | | | | | | | | |
| Taal | BEV | 16% | 8% | 33% | 8% | 0% | 22% | 2% | 0% | 4% |
| (BELOB) | Monotherapy | | | | | | | | | |
| Field | | 18% | NR | NR | 6% | NR | NR | 2% | NR | NR |
| (CABARET) | | | | | | | | | | |
| Heiland | | 12% | 10% | 38% | 0% | 0% | 22% | 0% | 0% | 10% |
| (Freiburg, | | | | | | | | | | |
| Germany) | | | | | | | | | | |
| Wick | | 14% | 10% | 25% | 9% | 10% | 9% | 8% | 10% | 9% |
| (EORTC | | | | | | | | | | |
| 26101 P2) | | | | | | | | | | |
| | | 6 mo Overall Survival | | | 9 mo Overall Survival | | | 12 Mo. Overall Survival | | |
| | | All | Un- | | All | Un- | | All | Un- | |
| Study | Treatment | Pts | methylated | Methylated | Pts | methylated | Methylated | Pts | methylated | Methylated |
| Current | MRZ + BEV | 75% | 68% | 78% | 55% | 45% | 78% | 39% | 15% | 67% |
| Study | | | | | | | | | | |
| Taal | BEV | 62% | 50% | 83% | 45% | 12% | 67% | 26% | 8% | 56% |
| (BELOB) | Monotherapy | | | | | | | | | |
| Field | | 61% | NR | NR | 39% | NR | NR | 24% | NR | NR % |
| (CABARET) | | | | | | | | | | |
| Heiland | | 18% | 25% | 58% | 30% | 12% | 40% | 10% | 12% | 24% |
| (Freiburg, | | | | | | | | | | |
| Germany) | | | | | | | | | | |

TABLE 29
Comparison of Marizomib Monotherapy with
Marizomib-Bevacizumab Combination Therapy

| | | PFS | | OS | |
|---|---|---|---|---|---|
| | | MRZ + | MRZ | MRZ + | MRZ |
| | | BEV | Mono | BEV | Mono |
| All Patients | # (# Censored) | 36 (6) | 30 (10) | 36 (13) | 30 (24) |
| | Median | 3.9 | 1.8 | 9.4 | NA |
| | 6 mo % | 34% | NA | 75% | 64% |
| | 9 mo % | 24% | NA | 60% | 64% |
| | 12 mo % | 10% | Na | 39% | NA |
| | 18 mo % | 0% | NA | 23% | NA |
| Unmethylated | # (# Censored) | 22 (1) | 18 (5) | 22 (3) | 18 (14) |
| MGMT | Median | 3.7 | 1.9 | 7.2 | NA |
| Promoter | 6 mo % | 36% | NA | 68% | 65% |
| | 9 mo % | 26% | NA | 45% | 65% |
| | 12 mo % | 10% | NA | 15% | NA |
| | 18 mo % | 0% | NA | 10% | NA |
| Methylated | # (# Censored) | 10 (3) | 7 (4) | 10 (6) | 7 (6) |
| MGMT | Median | 4.8 | 1.7 | NA | NA |
| Promoter | 6 mo % | 29% | NA | 78% | 83% |
| | 9 mo % | 15% | NA | 78% | 83% |
| | 12 mo % | 0% | NA | 67% | NA |
| | 18 mo % | 0% | NA | 53% | NA |

As shown above, the combination of treatment with marizomib and bevacizumab led to greater overall survival and progression free survival than treatment with bevacizumab or marizomib alone.

### Summary of All Patients Sorted by PFS

In Table 30 below, patients with bolded numbers have methylated MGMT promoters. Patients with italicized numbers are unknown or unequivocal.

TABLE 30
Summary of Patients sorted by PFS

| Pt | 1p19q | EGFR | EGFR | EGFRvIII | IDH1 | CDK4 | TP53 | PFS | OS |
|---|---|---|---|---|---|---|---|---|---|
| ID* | Deletion | Amplification | Mutation | Fusion | Mutation | Amplification | Mutation | (mo) | (mo) |
| 101- | No | Yes | No | No | No | No | No | 0.03 | 10.5 |
| 0402 | | | | | | | | | |
| 301- | No | Yes | No | Yes | No | No | No | 0.03 | 4.0 |
| 0413 | | | | | | | | | |
| 101- | No | No | A289D | No | No | No | No | 0.8 | 7.7 |
| 0419 | | | | | | | | | |
| 103- | No | No | No | No | No | No | No | 0.9 | 9.0 |
| 0422 | | | | | | | | | |
| 102- | No | No | No | No | No | Yes | No | 1.0 | 9.4 |
| 0104 | | | | | | | | | |
| 101- | | | | | | | | 1.2 | 2.7 |
| 0302 | | | | | | | | | |
| 102- | No | No | No | No | No | No | No | 1.4 | 4.7 |
| 0203 | | | | | | | | | |
| 103- | No | Yes | G598V | | | No | No | 1.5 | 1.5 |
| 0421 | | | | | | | | | |
| 301- | No | No | No | No | No | No | No | 1.6 | 3.5 |
| 0401 | | | | | | | | | |
| 103- | 1p | No | No | No | No | No | No | 1.6 | 6.0 |
| 0405 | | | | | | | | | |
| 102- | | | | | | | | 1.8 | 7.8 |
| 0415 | | | | | | | | | |
| 102- | No | No | No | No | No | No | S127Y | 1.9 | 5.2 |
| 0404 | | | | | | | | | |
| 301- | 19q | Yes | No | Yes | No | No | No | 2.5 | 6.0 |
| 0411 | | | | | | | | | |
| 101- | | | | | | | | 2.8 | 5.5 |
| 0423 | | | | | | | | | |
| 301- | No | No | P596L | No | No | No | C135R | 3.6 | 3.6 |
| 0406 | | | | | | | | | |
| 101- | No | Yes | A289V | No | E62fs | No | No | 3.6 | 5.7 |
| 0410 | | | | | | | | | |
| 102- | No | Yes | G598V | | No | No | | 3.6 | 6.4 |
| 0414 | | | | | | | | | |
| 101- | No | Yes | S768I | Yes | No | No | No | 3.6 | 7.2 |
| 0107 | | | | | | | | | |
| 101- | No | No | No | | No | No | No | 3.7 | 6.3 |
| 0202 | | | | | | | | | |
| 101- | No | Yes | No | No | No | Yes | No | 3.8 | 9.1 |
| 0101 | | | | | | | | | |
| 102- | No | No | No | No | No | No | No | 3.9 | 7.3 |
| 0403 | | | | | | | | | |
| 101- | | | | | | | | 4.4 | 5.4 |
| 0416 | | | | | | | | | |
| 101- | No | No | No | No | No | Yes | H179Y | 4.8 | 9.2 |
| 0407 | | | | | | | | | |
| 301- | 19q | No | No | No | No | No | N235S | 4.9 | 6.0 |
| 0412 | | | | | | | | | |
| 301- | No | No | No | No | No | No | | 5.4 | 8.8 |
| 0418 | | | | | | | | | |
| 101- | No | No | No | No | No | No | No | 5.5 | 8.7 |
| 0409 | | | | | | | | | |
| 301- | No | Yes | R324L | No | No | Yes | No | 7.2 | 9.4 |
| 0424 | | | | | | | | | |
| (on study) | | | | | | | | | |
| 102- | No | Yes | No | No | No | No | No | 7.6 | 9.0 |
| 0417 | | | | | | | | | |
| 102- | 1p | | | No | | | | 7.2 | 7.2 |
| 0420 | | | | | | | | | |
| 101- | | | | | | | | 7.0 | 9.2 |
| 0408 | | | | | | | | | |
| 101- | | | | | | | | 7.3 | 11.4 |
| 0105 | | | | | | | | | |
| 102- | 1p | Yes | No | Yes | No | No | No | 9.7 | 16.4 |
| 0106 | | | | | | | | | |
| 101- | No | No | A767_P772 | No | No | Yes | No | 10.4 | 10.4 |
| 0303 | | | duplication | | | | | | |
| 301- | 1p & | No | No | | No | No | P151S | 10.7 | 10.7 |
| 0301 | 19q | | | | | | | | |
| 301- | | | | | | | | 13.5 | 15.0 |
| 0201 | | | | | | | | | |
| 101- | 19q | No | No | No | No | No | No | 15.0 | 17.5 |
| 0103 | | | | | | | | | |

### Pharmacokinetic and Pharmacodynamic Parameters.

Table 31 shows a summary of the pharmacokinetic and pharmacodynamic parameters for marizomib and bevacizumab.

TABLE 31
Pharmacokinetic and Pharmacodynamic Summary

| | | | |
|---|---|---|---|
| Parameter (Units) | 0.55 mg/m² | 0.7 mg/m² | 0.8 mg/m² |

Marizomib PK determined on C1D8

| | | | |
|---|---|---|---|
| T_{1/2} (min) | 8.2 ± 0.8 (4) | 16.0 ± 8.08 (3) | 7.27 ± 0.423 (3) |
| Tₘₐₓ (min) | 15.5 ± 1.4 (6) | 21.3 ± 1.3)0 (3) | 8.0 ± 0 (3) |
| Cₘₐₓ (ng/mL) | 23.1 ± 11.3 (6) | 64.9 ± 1.73 (3) | 26.5 ± 7.92 (3) |
| AUCₗₐₛₜ (min*ng/mL) | 265 ± 101 (6) | 193 ± 85 (2) | 392 ± 115 (3) |
| Vd (L) | 54.4 ± 10.6 (4) | 48.3 ± 16.1 (3) | 55.0 ± 19.7 (3) |
| CL_{obs} (L/hr) | 297 ± 73.0 (4) | 272 ± 166 (3) | 304 ± 85.0 (3) |

BEV PK determined on C1D1 & C1D15

| | | | |
|---|---|---|---|
| Cₘₐₓ D 1 (μg/mL) | 275 ± 37.5 (6) | 193 ± 8 (2) | 267 ± 13.3 (3) |
| Cₘᵢₙ D 15 (μg/mL) | 89.7 ± 6.2 (6) | 81.1 ± 17.4 (2) | 85.0 ± 9.87 (3) |
| Cₘₐₓ D 15 (μg/mL) | 351 ± 46.6 (5) | 402 ± 123 (3) | 380 ± 56.5 (2) |

Proteasome subunit inhibition in PWB
post-Marizomib infusion (peak effect)

| | | | |
|---|---|---|---|
| % CT-L inhibition | 100 ± 0 (5) | 100 ± 0 (3) | 100 ± 0 (3) |
| % T-L inhibition | 52.0 ± 8.0 (5) | 77.3 ± 10.5 (3) | 69.3 ± 7.2 (3) |
| % C-L inhibition | 21.0 ± 7.8 (5) | 50.4 ± 9.1 (3) | 51.9 ± 8.3 (3) |

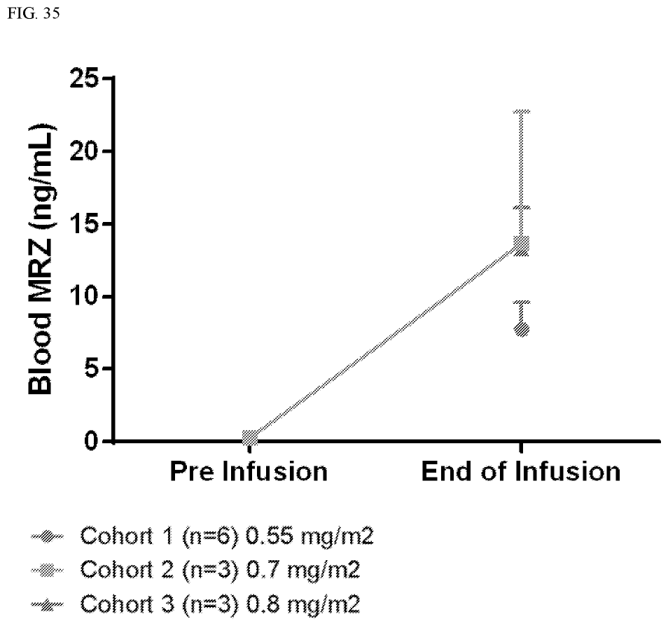

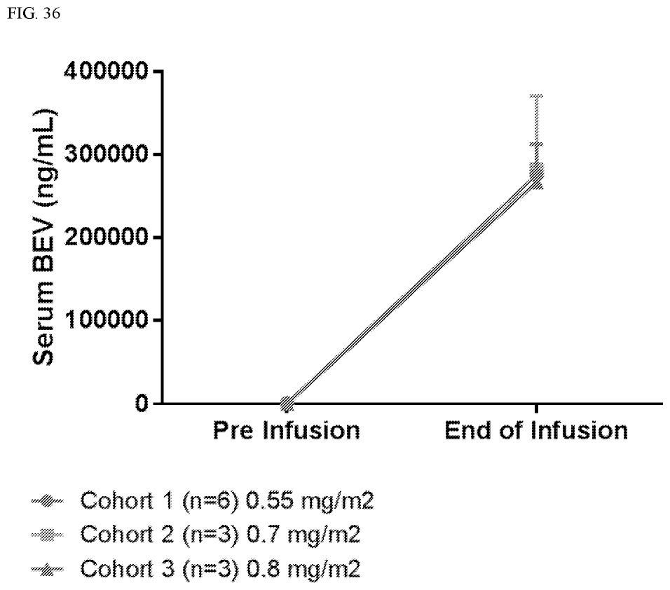

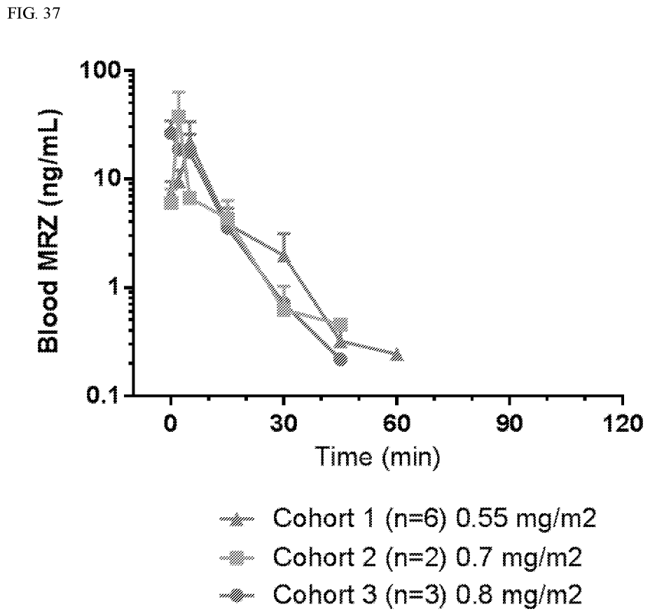

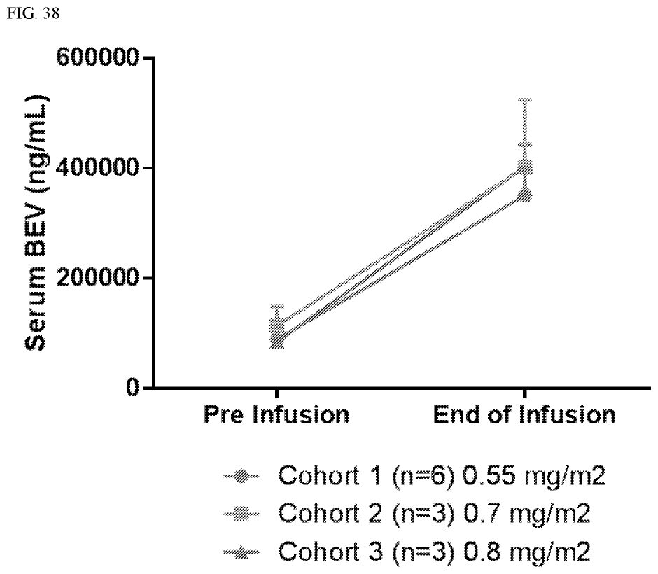

FIG. 35 shows the concentration of marizomib in the blood of patients on C1D1 pre- and post-infusion. FIG. 36 shows the concentration of bevacizumab in the serum of patients C1D1 pre- and post-infusion. FIG. 37 shows the concentration of marizomib in the blood as a function of time on C1D8. FIG. 38 shows concentration of bevacizumab in serum pre- and post-infusion for different cohorts on C1D15.

As set forth in FIGS. 35-38, the mean Cₘₐₓ of bevacizumab across all dose cohorts was 275 μg/mL on Day 1; the mean Cₘᵢₙ of bevacizumab on Day 15 was 95 μg/mL; and the mean Cₘₐₓ of bevacizumab on Day 1 was 379 μg/mL. The results agree with published literature precedent for Cₘₐₓ of bevacizumab of 284 μg/mL at Day 0 for a 10 mg/kg dose (Gordon et al., 2001).

### Example 2

### Phase 1, Multicenter, Open-Label, Dose-Escalation, Combination Study of Marizomib and Bevacizumab in Bevacizumab-Naiive Subjects with WHO Grade IV Malignant Glioma Followed by Phase 2 Trials of Single Agent Marizomib and Combination Marizomib and Bevacizumab

Example 2 represents updates to the procedure and protocol set forth in Example 1. Protocol Synopsis Title

A Phase 1, Multicenter, Open-label, Dose-escalation, Combination Study of Marizomib and Bevacizumab in Bevacizumab-Naive Subjects with WHO Grade IV Malignant Glioma Followed by Phase 2 Trials of Single Agent Marizomib and Combination Marizomib and Bevacizumab

### Indication

WHO Grade IV Malignant Glioma (G4 MG) in bevacizumab-naive subjects Background and Study Rationale

The study population includes subjects with G4 MG (including glioblastoma and gliosarcoma) who are in first or second relapse and who have not previously received any bevacizumab (BEV) or other anti-angiogenic agents, including sorafenib, sunitinib, axitinib, pazopanib, everolimus, or cilengitide, or marizomib (MRZ) or any other proteasome inhibitor, including bortezomib (BTZ), carfilzomib (CFZ), or ixazomib (IXZ).

One of the few treatment options currently approved for recurrent G4 MG is BEV with a 6-month progression-free survival (PFS) rate of ˜43% and median overall survival (OS) of ˜9 months ( ). Additional treatment options are needed for these subjects. The Phase 1 (Part 1) portion of this study suggested activity of the combination of BEV and MRZ. The Phase 2 (Part 2) portion of the study explored the single agent activity of MRZ. The Part 3 portion of the study explores the activity of the combination of MRZ and BEV, using an intrapatient dose-escalation dosing regimen, in a Phase 2 setting.

Published literature indicates that targeting the proteasome in glioma cells has shown significant anti-tumor activity ( ). In vitro studies of multiple glioma cell lines were highly sensitive to MRZ. MRZ has relatively little effect on neural stem/progenitor cells suggesting minimal neurotoxicity while severely affecting both malignant glioma stem cells and glioma cell lines. MRZ potently and robustly inhibited migration and invasion of human glioblastoma (GBM) cell lines in two different assays. Treatment with MRZ decreased matrix invasion by either of two GBM lines by approximately 90%, a highly significant effect. Preclinical data demonstrate that MRZ crosses the blood brain barrier 0. In addition, preclinical studies have demonstrated proteasome inhibition with BTZ stimulates VEGF levels suggesting that there may be a synergy combining proteasome inhibitors with VEGF inhibitors ( ).

The preliminary clinical data with respect to tolerability and early evidence of promising activity of MRZ with or without dexamethasone has been shown in results from ongoing Phase 1 clinical studies in subjects with advanced solid tumors, refractory non-Hodgkin's lymphoma, and relapsed/refractory multiple myeloma.

Based on these pre-clinical and clinical data, the addition of MRZ to BEV could be a promising combination regimen in recurrent GBM.

Part 1 (Phase 1) established 0.8 mg/m² MRZ plus BEV (fixed dose, 10 mg/kg) to be the recommended Phase 2 dose (RP2D), however the MTh as per protocol definition was not reached. Part 2 was conducted to determine the contribution towards efficacy of MRZ to the combination by determining the single agent activity of MRZ in a Phase 2 setting.

Analysis of ongoing safety and efficacy data of patients in the Part 1 (Phase 1) portion of the study suggest that doses that cause central nervous system (CNS) adverse events (AEs) appear to be more active than those that do not. The Part 3 (Phase 2) portion of the study (added with Amendment 3) is to determine point estimates for objective response rate (ORR), PFS, and OS for patients who receive the combination of MRZ and BEV with MRZ titrated to toxicity on an individual patient basis (intrapatient dose escalation).

### Objectives

Part 1 Phase 1

Primary Objective

To determine the maximum tolerated dose (MTh) or Maximum Administered Dose (MAD) and recommended Phase 2 dose (RP2D) of the combination of marizomib (MRZ) +bevacizumab (BEV) with MRZ as a once weekly dose for 3 weeks of a 28-day cycle and with a fixed dose and schedule of BEV (10 mg/kg administered on Days 1 and 15) in subjects with progressive or recurrent G4 MG, who have not previously been treated with either an anti-angiogenic agent including but not limited to, BEV or a proteasome inhibitor including, but not limited to, MRZ.

Secondary Objectives

To evaluate the safety of the combination of MRZ+BEV in the subject population.

To evaluate activity of the combination of MRZ+BEV in the subject population including:
Radiographic Overall Response Rate (ORR) (RANO 2010 criteria)
Progression-free Survival (PFS)
Overall Survival (OS)

To evaluate the pharmacokinetics (PK) of MRZ and BEV when administered in combination in the subject population.

To assess the blood proteasome inhibition pharmacodynamic (PD) activity of the combination of MRZ+BEV in the subject population.

Exploratory Objectives

To evaluate baseline tumor proteasome activity, gene signature and transcriptional profiling using pre-study, archived tissue samples.

To evaluate neurological coordination assessment using the Scale for the Assessment and Rating for Ataxia (SARA).

To evaluate Quality of Life Assessments using Functional Assessment of Cancer Therapy (FACT) questionnaires:
FACT-Cognitive Function (FACT-Cog)
FACT-Brain (FACT-Br)

Part 2 Phase 2

Primary Objective

To assess the activity of a once weekly dose for 3 weeks of a 28-day cycle of MRZ in subjects with progressive or recurrent G4 MG, who have not previously been treated with either an anti-angiogenic agent or a proteasome inhibitor.

Secondary Objectives

To evaluate the safety of single agent MRZ in the subject population.

Exploratory Objectives

To evaluate baseline tumor proteasome activity, gene signature and transcriptional profiling using pre-study, archived tissue samples.

To evaluate neurological coordination assessment using the Scale for the Assessment and Rating for Ataxia (SARA).

To evaluate Quality of Life Assessments using Functional Assessment of Cancer Therapy (FACT) questionnaires:

FACT-Cognitive Function (FACT-Cog)

FACT-Brain (FACT-Br)

Part 3 Phase 2

Primary Objective

To assess the activity of the combination of once weekly MRZ dosing for 3 weeks (allowing for intra-patient dose escalation) and every other week dosing of BEV at 10 mg/kg in 28-day cycle in subjects with progressive or recurrent G4 MG, who have not previously been treated with either an anti-angiogenic agent or a proteasome inhibitor.

Secondary Objectives

To evaluate the safety of combination of MRZ with intrapatient dose escalation and BEV at a fixed dose in the subject population.

Exploratory Objectives

To evaluate baseline tumor proteasome activity, gene signature and transcriptional profiling using pre-study, archived tissue samples.

To evaluate neurological coordination assessment using the Scale for the Assessment and Rating for Ataxia (SARA).

To evaluate Quality of Life Assessments using Functional Assessment of Cancer Therapy (FACT) questionnaires:

FACT-Cognitive Function (FACT-Cog)

FACT-Brain (FACT-Br)

Study Design

Part 1 of this protocol is a Phase 1, open-label, 3+3, dose-escalation study in subjects with WHO Grade IV Malignant Glioma (G4 MG) who are in first or second relapse and who have not previously received any BEV or other anti-angiogenic agent, including sorafenib, sunitinib, axitinib, pazopanib, everolimus, or cilengitide or MRZ or any other proteasome inhibitor, including BTZ, CFZ, or IXZ. Three to 6 evaluable subjects per cohort will be enrolled: approximately 24 subjects to determine the MTD or MAD (Part 1 Dose-escalation) and an addition of at least 12 more subjects to confirm the MTD/MAD and determine the RP2D (Part 2 Expansion Cohort) and assess preliminary activity to a total of up to 36 subjects. Subjects may not be enrolled in more than 1 cohort.

The Phase 1 portion will be followed by Part 2, a Phase 2 portion of the trial of single agent MRZ administered as a 10-minute infusion at a dose of 0.8 mg/m² (the RP2D from Phase 1) every week for 3 weeks in 28-day cycles. This portion of the trial will be conducted as a 2-stage sequential design of up to 30 response-evaluable patients.

The Part 2 Phase 2 portion of the trial will be followed by Part 3, a Phase 2 study of combination MRZ using intra-patient dose escalation and BEV at a fixed dose. MRZ will be administered as a 10-minute infusion every week for 3 weeks in 28-day cycles at a starting dose of 0.8 mg/m² (the RP2D from Part 1 Phase 1). After the first cycle without a dose-limiting adverse event (DLAE), the dose of MRZ will be increased to 1.0 mg/m² and after 1 more cycle without a DLAE the dose of MRZ will be increased to 1.2 mg/m². BEV will be administered every 2 weeks (Days 1 and 15 of each 28-day cycle) at a fixed dose of 10 mg/kg.

DLAEs are MRZ-related AEs 1) related to disturbances in the cerebellum (i.e., ataxia, dizziness, dysarthria, fall, gait disturbances) plus hallucinations of any grade or 2) Grade ≥2 other AEs. This portion of the trial will be conducted in approximately 40 eligible patients of which, based on the AEs seen in Part 1 of the study, about 24 patients are expected to be eligible for intra-patient dose escalation.

Study Treatments

MRZ is an investigational product that will be provided by the Sponsor. BEV is available commercially and will be provided by the Investigator via prescription to subjects who are enrolled into the Phase 1 portion of this study.

Study Treatment

Part 1 Phase 1

All subjects will receive intravenous (IV) MRZ infusion followed by IV BEV infusion as follows:

IV MRZ will be administered as a 10-minute (or longer) IV infusion on Days 1, 8, and 15 of every 28-day cycle. IV hydration will be given both before and after the infusion.

IV BEV will be administered as an IV infusion (90 minutes 1st dose, 60 minutes 2nd dose and 30 minutes afterward assuming tolerability) at a dose of 10 mg/kg on Days 1 and 15 of every 28-day cycle. BEV will be administered approximately 10 minutes after the end of the MRZ infusion when co-administered on the same day.

Part 2 Phase 2

All subjects will receive IV MRZ infusion.

MRZ will be administered as a 10-minute, IV infusion on Days 1, 8, and 15 of every 28-day cycle. IV hydration will be given before the infusion.

Part 3 Phase 2

All subjects will receive IV MRZ infusion and IV BEV infusion.

MRZ will be administered as a 10-minute, IV infusion on Days 1, 8, and 15 of every 28-day cycle using intra-patient dose escalation. Starting dose will be 0.8 mg/m² (RP2D dose). Assuming the starting dose was tolerated and no DLAE was observed, the dose will be increased to 1.0 mg/m² after 1 cycle. Assuming the increased dose was tolerated again and no DLAE was observed, the dose of MRZ will be increased to 1.2 mg/m² after 1 cycle. Dose reductions will be applied as necessary and according to the toxicities noted.

If the starting dose is not tolerated (after appropriate medical treatment of adverse events, if applicable, the dose will be decreased to 0.7 mg/m². A further reduction to 0.55 mg/m² is allowed, if necessary.

BEV will be administered as an IV infusion (90 minutes 1st dose, 60 minutes 2nd dose and 30 minutes afterward assuming tolerability) at a fixed dose of 10 mg/kg on Days 1 and 15 of every 28-day cycle. BEV will be administered approximately 10 minutes after the end of the MRZ infusion when co-administered on the same day. Dose reductions of BEV will not be made, but dose delay or discontinuation will be made depending upon the observed adverse events.

Dose-Limiting Toxicity Part 1 Phase 1 (only)

For the Phase 1 portion of the trial, dose-limiting toxicity (DLT) is defined as the occurrence of any of the following adverse events (AEs) related to study treatment observed during Cycle 1, using National Cancer Institute Common Terminology Criteria for Adverse Events version 4.03 (NCI-CTCAE v 4.03) to determine severity:

Grade 3 thrombocytopenia or Grade 2 thrombocytopenia with bleeding.

Grade 4 neutropenia or anemia lasting for more than 4 days.

Febrile neutropenia.

Any ≥Grade 2 neurological event lasting more than 4 days.

Grade 3 or 4 non-hematological toxicity (excluding alopecia) lasting for more than 4 days despite adequate supportive therapy or preventing the next scheduled dose from being administered within 4 days of scheduled day; for ≥Grade 3 fatigue to be considered a DLT, it must be present for more than 7 days.

Subjects without DLT in Cycle 1 who do not receive 3 MRZ doses or 2 BEV doses within 5 weeks from first dose will not be evaluable for DLT and will be replaced.

Part 1 Phase 1 Dose Escalation Subjects who have completed Screening procedures and meet all eligibility criteria may be enrolled into the study.

A 3+3 design will be used to define the MTD/MAD for MRZ+BEV combination treatment in 28 day cycles, with MRZ administered on Days 1, 8, and 15 and BEV on Days 1 and 15.

MRZ dosing will begin at 0.55 mg/m² once weekly (Cohort 1). Additional dose cohorts are planned as shown below (Table 32):

TABLE 32
Dose Cohorts for MRZ + BEV Combination

| | IV MRZ | IV BEV |
|---|---|---|
| Cohort | Days 1, 8, and 15 | Days 1 and 15 |
| −2 | 0.3 mg/m² | 10 mg/kg |
| −1 | 0.4 mg/m² | 10 mg/kg |
| 1 | 0.55 mg/m² | 10 mg/kg |
| 2 | 0.7 mg/m² | 10 mg/kg |
| 3 | 0.8 mg/m² | 10 mg/kg |
| 4 | Additional cohorts with extended | 10 mg/kg |
| | infusion duration if required | |

Initially 3 subjects will be enrolled into a cohort, commencing with Cohort 1 and the doses shown in Table 32 above. Dose escalation will proceed as follows:

If none of the first 3 evaluable subjects in a dose cohort experience a DLT during Cycle 1, then enrollment into the next dose cohort can be initiated.

If ≥2 of the first 3 evaluable subjects in a dose cohort experience a DLT during Cycle 1, then the MTD has been exceeded and dose escalation will not proceed.

If 1 of the first 3 evaluable subjects in a dose cohort experiences a DLT during Cycle 1, then an additional 3 subjects will be enrolled into the same cohort.

If 1/6 evaluable subjects in the expanded 6-subject cohort experiences a DLT during Cycle 1, then the next higher dose cohort can be tested and enrollment of the next 3 subjects at the next higher dose level can be initiated.

If ≥2/6 evaluable subjects in the expanded 6-subject cohort experience a DLT during Cycle 1, then the MTD has been exceeded and no further dose escalation will occur.

The MTD is defined as the dose level below the cohort where DLT is observed in at least 2 subjects in the same cohort during Cycle 1. Intermediate dosing levels may be explored if indicated. Additional cohorts starting below the MTD for the 10-minute infusion may be enrolled to explore extended infusion lengths. The dose of 0.8 mg/m² will not be exceeded and will be the MAD. The RP2D is the MTD/MAD unless further safety information suggests a lower dose for future trials.

Once the MTD or Maximum Administered Dose (MAD) has been identified, a cohort of at least 12 additional, evaluable subjects will be treated at the MTD/MAD to further confirm the safety and to assess preliminary activity for the combination treatment. This cohort may be used to determine the RP2D.

Part 2 Phase 2 Dose Escalation Dose escalation of MRZ was not allowed in this portion of the study.

Part 3 Phase 2 Intrapatient Dose Escalation MRZ dosing will start at 0.8 mg/m² given on Days 1, 8, and 15 in 28 day cycles as a 10 minute IV infusion. If the patient tolerates the MRZ dose during the first cycle without DLAE, the dose of MRZ will be increased to 1.0 mg/m² and after 1 more cycle without a DLAE, the dose of MRZ will be increased to 1.2 mg/m².

DLAEs are MRZ-related AEs 1) related to disturbances in the cerebellum (i.e., ataxia, dizziness, dysarthria, fall, gait disturbances) plus hallucinations of any grade or 2) Grade ≥2 other AEs.

If the starting dose is not tolerated after appropriate medical treatment of AEs in the first cycle, then the dose will be decreased to 0.7 mg/m² with no further dose increases allowed.

BEV will be administered as an IV infusion (90 minutes 1st dose, 60 minutes 2nd dose and 30 minutes afterward assuming tolerability) at a dose of 10 mg/kg on Days 1 and 15 of every 28-day cycle. BEV will be administered approximately 10 minutes after the end of the MRZ infusion when co-administered on the same day. The dose of BEV will not increase. No dose adjustments will be made to BEV dosing, although doses may be delayed or discontinued.

No. Subjects Part 1 Phase 1: 36 subjects were enrolled in the study at multiple centers.

Part 2 Phase 2: Up to 30 response-evaluable subjects will be enrolled in the study at multiple centers.

Part 3 Phase 2: Up to 40 eligible subjects will be enrolled in the study at multiple centers.

Study Population

The study population includes subjects with G4 MG (including glioblastoma and gliosarcoma) who are in first or second relapse and who have not previously received any BEV or other anti-angiogenic agent, including sorafenib, sunitinib, axitinib, pazopanib, everolimus, or cilengitide or MRZ or any other proteasome inhibitor, including BTZ, CFZ, or IXZ. The eligibility criteria are the same for both Phase 1 and Phase 2 portions of the trial except where noted.

Inclusion Criteria

Subjects must meet the following criteria to be eligible for study participation:

Understand and voluntarily sign and date an informed consent document prior to any study related assessments/procedures are conducted.

Males and females of age ≥18 years at the time of signing of the informed consent document.

All subjects must have histologic evidence of G4 MG (including glioblastoma and gliosarcoma) and radiographic evidence of recurrence or disease progression (defined as either a greater than 25% increase in the largest bidimensional product of enhancement, a new enhancing lesion, or significant increase in T2 FLAIR). Subjects must have at least 1 measurable lesion by RANO criteria (≥10 mm in 2 perpendicular diameters).

Subjects must have previously completed standard radiation therapy and been exposed to temozolomide. Patients must be in first or second relapse.

Subjects with archival tumor tissue suitable for proteasome activity and genetic testing must give permission to access and test the tissue; subjects without archival tumor tissue are eligible.

No prior treatment with MRZ or any other proteasome inhibitors, including BTZ, CFZ, or IXZ or BEV or any other anti-angiogenic agents, including sorafenib, sunitinib, axitinib, pazopanib, everolimus, or cilengitide.

No investigational agent within 4 weeks prior to first dose of study drug.

At least 4 weeks from surgical resection and at least 12 weeks from end of radiotherapy prior to enrollment in this study, unless relapse is confirmed by tumor biopsy or new lesion outside of radiation field, or if there are two MRIs confirming progressive disease that are ˜8 weeks apart.

Subjects with a history of seizures must be on a stable dose of anti-epileptic drugs (AEDs) and without seizures for 14 days prior to enrollment in patients enrolled prior to Amendment 2. Subjects enrolled after Amendment 2 is approved with a history of seizures must be on a stable dose of anti-epileptic drugs (AEDs) for 7 days prior to enrollment.

All AEs resulting from prior chemotherapy, surgery, or radiotherapy, must have resolved to NCI-CTCAE (v. 4.03) Grade □1 (except for laboratory parameters outlined below).

Laboratory results within 7 days prior to MRZ administration (transfusions and/or growth factor support may not be used to meet this criteria):

Platelet count≥100×109/L.

Hemoglobin≥9 g/dL.

Absolute neutrophil count (ANC)≥1.5×109/L/

Serum bilirubin≤1.5×upper limit of normal (ULN) or ≤3×ULN if Gilbert's disease is documented.

Aspartate transaminase (AST)≤2.5 ULN.

Alanine transaminase (ALT)≤2.5 ULN.

Serum creatinine≤1.5×ULN.

Urine protein: creatinine ratio≤1.0 at screening.

Karnofsky Performance Status (KPS) score≥70%.

For women of child-bearing potential and for men with partners of child-bearing potential, subject must agree to take contraceptive measures for duration of treatments and for 3 months after the last dose of MRZ and 6 months after the last dose of BEV, whichever is longer.

Willing and able to adhere to the study visit schedule and other protocol requirements.

Exclusion Criteria:

Subjects with any of the following will be excluded from participation in the study:

Co-medication that may interfere with study results, e.g., immuno-suppressive agents other than corticosteroids. (Steroid therapy for control of cerebral edema is allowed at the discretion of the Investigator. Subjects should be on a stable dose of steroids for at least 1 week prior to first dose of MRZ.)

Evidence of CNS hemorrhage on baseline MRI or CT scan (except for post-surgical, asymptomatic Grade 1 hemorrhage that has been stable for at least 3 months for subjects enrolled prior to Amendment 2 and for at least 4 weeks in subjects enrolled after Amendment 2 is approved).

History of thrombotic or hemorrhagic stroke or myocardial infarction within 6 months.

Chemotherapy administered within 4 weeks (except 6 weeks for nitrosoureas, 12 weeks for an implanted nitrosoureas wafer, and 1 week from metronomic chemotherapy, like daily temozolomide and etoposide) prior to Day 1 of study treatment, unless the subject has recovered from all expected toxicities from the chemotherapy.

Pregnancy or breast feeding.

Uncontrolled intercurrent illness including, but not limited to, ongoing or active infection requiring IV antibiotics & psychiatric illness/social situations that would limit compliance with study requirements, or disorders associated with significant immunocompromised state.

Known previous/current malignancy requiring treatment within ≤3 years except for cervical carcinoma in situ, squamous or basal cell skin carcinoma, and superficial bladder carcinoma.

Any comorbid condition that confounds the ability to interpret data from the study as judged by the Investigator or Medical Monitor.

BEV-Specific Concerns (Note: These exclusion criteria also apply to the Part 2 Phase 2 portion of the study even though BEV is not administered so that the patient populations among Part 1, Part 2, and Part 3 are similar):

Any prior history of hypertensive crisis or hypertensive encephalopathy.

Systolic blood pressure (BP)>150 mmHg or diastolic BP>100 mmHg.

Unstable angina.

New York Heart Association Grade≥II congestive heart failure.

History of myocardial infarction within 6 months.

Subjects with mean QTcF interval>500 ms.

Clinically significant peripheral vascular disease

Evidence of bleeding diathesis, coagulopathy as documented by an elevated (≥1.5×ULN) prothrombin time (PT), partial thromboplastin time (PTT), or bleeding time. The use of full-dose oral or parenteral anticoagulants is permitted as long as the PT or aPTT is within therapeutic limits (according to the medical standard of the enrolling institution) and the subject has been on a stable dose of anticoagulants for at least 2 weeks prior to the first study treatment.

Major surgical procedure, open biopsy, or significant traumatic injury within 28 days prior to Day 1 or anticipation of need for major surgical procedure during course of the study.

Minor surgical procedures, fine needle aspirations or core biopsies within 7 days prior to Day 1.

History of abdominal fistula, GI perforation, or intra-abdominal abscess within 6 months prior to Day 1.

Serious, non-healing wound, ulcer, or bone fracture requiring surgical intervention

Length of Study Participation

Subjects may continue on study treatment until disease progression, unacceptable toxicity, withdrawal of consent, or termination of the study. For subjects who discontinue study drug for reasons other than disease progression, whenever possible, tumor assessment will continue as per protocol until disease progression. After disease progression, subjects will be followed for survival and the start of first new anti-GBM therapy and its outcome.

Investigational Product/Background Therapy/Route/Regimen

Part 1 Phase 1

MRZ will be administered IV over 10 minutes. Other infusion lengths may be explored. Volume of administration will vary based on assigned dose (Table 32) and subject body surface area (BSA). To mitigate the possibility of renal dysfunction, subjects will receive normal saline administered at 350 mL/hour for 1 hour before and for 2 hours after the MRZ infusion. The MRZ infusion will be started after approximately 350 mL of saline have been given over 1 hour. After the MRZ infusion has been completed, approximately 700 mL of saline will be given over 2 hours, for a total volume of saline infusion equal to approximately 1 L. Post infusion hydration may be reduced at the discretion of the Investigator. The lyophilized drug product contains 2 mg API and 60 mg sucrose bulk excipient. Cartons contain one vial of lyophile together with a Diluent vial containing 55% propylene glycol, 5% ethanol, and 40% citrate buffer pH 5 (20 mL fill; 10 mL intended for use).

BEV will be administered as an IV infusion (90 minutes 1st dose, 60 minutes 2nd dose and 30 minutes afterward assuming tolerability) as described in the current package insert. BEV will be administered approximately 10 minutes after the end of the MRZ infusion when co-administered on the same day.

Part 2 Phase 2

MRZ will be administered IV over 10 minutes at a dose of 0.8 mg/m². To mitigate the possibility of renal dysfunction, subjects will receive normal saline administered at 250 mL for 30 minutes before the MRZ infusion. The lyophilized drug product is the same as used in the Phase 1 portion.

Part 3 Phase 2

MRZ will be administered IV over 10 minutes at a starting dose of 0.8 mg/m². Based on the patient's tolerability, the dose of MRZ may be increased after Cycles 1 and 2. The lyophilized drug product is the same as used in the other portions of the study. For this part of the protocol, hydration prior to the MRZ dose is not required.

BEV will be administered as an IV infusion (90 minutes 1st dose, 60 minutes 2nd dose and 30 minutes afterward assuming tolerability) as described in the current package insert. BEV will be administered approximately 10 minutes after the end of the MRZ infusion when co-administered on the same day.

Procedures

Study visits and procedures will be performed as outlined in Table 33. The study will consist of Screening, Baseline, Treatment, and Follow-up periods.

Screening

The screening period may not exceed a 28-day window (with an extra 3 day window for unavoidable delays) prior to start of study treatment (Cycle 1 Day 1). Assessments will include medical history, cancer history including previous treatments, and tumor assessments. Tumor assessment must have a baseline MRI scan with contrast within 14 (+3) days prior to first treatment with investigational product.

Baseline

Physical examination including Karnofsky Performance Status (KPS), neurological evaluation, neurological coordination assessment using the Scale for the Assessment and Rating of Ataxia (SARA), quality of life assessment using the FACT-Cog and FACT-Br, vital signs measurement, electrocardiogram (ECG), and laboratory tests are to be conducted within 7 days prior to Cycle 1 Day 1.

Treatment

Subjects may continue on study treatment until disease progression, unacceptable toxicity, withdrawal of consent, or termination of the study. Assessment will include MRI scans at the end of every even numbered cycle (±7 days) using RANO 2010 criteria for assessment. Responses (complete response [CR] and partial response [PR]) should be confirmed by repeat scans performed 4 weeks (±2 days) later.

Functional status using the KPS, neurological coordination assessment using the SARA, and quality of life assessment using the FACT-Cog and FACT-Br will be assessed regularly.

Subjects who discontinue study drug for reasons other than disease progression whenever possible will continue tumor assessment as per protocol schedule until progression.

End-of-Treatment Visit

Subject will be followed for safety for 28 (+7) days after discontinuation of trial therapy (Part 1 Phase 1 and Part 3 Phase 2: both MRZ and BEV; Part 2 Phase 2: MRZ).

Post Study Follow-up

All subjects will be followed in the long-term survival follow-up period for as long as they are alive. Long-term follow up will occur every 3 months (±7 days) after the End-of-Treatment visit. Telephone contact will be sufficient to document survival status. During the follow-up period, the following information will be collected: survival, and first subsequent anti-malignant glioma regimens (regimen, start and end date, and treatment outcome).

Overview of Assessments Activity (Efficacy) Assessments

Tumor response, including progressive disease, will be assessed with MRI every 2 cycles (at the end of each even-numbered cycle of therapy) according to the RANO 2010 criteria, including:

Radiographic Response Rate

Progression-free Survival (PFS)

Overall Survival (OS)

Pharmacokinetic Assessments: MRZ (Part 1 Phase 1 only)

Blood samples will be taken for peak and trough measurements, pre-dose and immediately prior to (end of infusion) EOI, on Cycle 1 Day 1. On Cycle 1 Day 15 full PK sampling will be done: pre-dose, immediately prior to EOI and then 2, 5, 15, 30, 45, 60, 90 and 120 minutes post infusion. The following PK parameters will be estimated by non-compartmental analysis:

Maximum observed blood drug concentration (Cₘₐₓ)

Time of maximum blood concentration (tₘₐₓ)

Elimination half-life (t1/2)

Area under the blood concentration-time curve (AUC₀₋₄, AUC_{0-inf})

Clearance (CL)

Volume of distribution (Vd)

Pharmacokinetic Assessments: BEV (Part 1 Phase 1 only)

Pre-dose and immediately prior to EOI serum samples will be taken on Cycle 1 Days 1 and 15 to assess BEV peak and trough levels in plasma.

Blood Pharmacodynamic Assessments (Part 1 Phase 1 only)

Change in proteasome activities in packed whole blood (PWB) lysates and peripheral blood mononuclear cell (PBMC) lysates, comparing pre-drug and post drug levels on Days 1, 8, and 15 of Cycle 1; Days 1 and 15 of each Cycle thereafter; and at the End-of Treatment Visit.

Tumor Biomarker Assessments

(Exploratory) Assessment of pre-treatment proteasome activity, genomic analysis and transcriptional profiling in flash-frozen and/or formalin fixed paraffin embedded, archived subject tumor sample tissue (at the discretion of the Sponsor), and if archived tumor tissue is available.

Statistical Analyses Overview

Part 1 Phase 1

A 3+3 design will be utilized to determine the MTD/MAD for MRZ+BEV combination treatment in 28-day cycles. (Subjects who do not have a DLT will be replaced if they discontinue treatment with MRZ or BEV in Cycle 1 for any other reasons.) After MTD/MAD has been determined in the dose-escalation part of the study, at least 12 additional subjects will be treated at the MTD/MAD to confirm the safety and assess the preliminary activity for the combination of MRZ+BEV.

For all analyses by dose cohorts, the MTD/MAD confirmation cohort subjects will be combined with the corresponding dose cohort in the MTD/MAD determination phase as one single dose cohort.

Part 2 Phase 2

A 2-stage sequential design will be utilized in Phase 2. Fifteen response-evaluable patients will be in the first stage. If at least 1 response is observed, then the trial will be expanded, and an additional 15 response-evaluable patients will be treated. If at least 5 responses are observed in the 30 response-evaluable patients, then MRZ will be considered active as a single agent.

Part 3 Phase 2

Forty eligible patients will be treated. Assuming there are 30 deaths observed (i.e., 25% of the subjects are censored), the resulting 95% confidence interval (CI) is 7.2-14.8 months, with a width equal to 7.6 months for an estimated median survival of 10 months.

Activity (All Parts)

Tumor response, including PD activity, progression-free survival (PFS), and overall survival (OS) will be assessed. Tumor response will be assessed by the Investigators using RANO 2010 criteria. The overall confirmed response rate will be presented. The response rate, PFS, and OS will also be tabulated by dose cohorts in Part 1 Phase 1 and for all response-evaluable patients in Part 2 Phase 2 and Part 3 Phase 2. Endpoints of response based on tumor assessments will be calculated for subjects who received at least 3 doses of MRZ and had at least 1 post-dose tumor evaluation.

Safety (All Parts)

All subjects will be evaluated for safety analysis if they receive at least one dose of MRZ or BEV in Phase 1 or MRZ in Phase 2. The safety data will be presented in individual listings and summary tables, including frequency tables for adverse events and frequency and shift tables for laboratory variables. The safety population will be all subjects who received at least one dose of either study drug in Phase 1 or MRZ in Phase 2.

Pharmacokinetics (PK) (Part 1 Phase 1 only)

Non-compartmental analyses will be performed. The following PK parameters will be calculated using standard non-compartmental analysis: maximum observed blood drug concentration (Cₘₐₓ), time of maximum blood concentration (Tₘₐₓ), elimination half-life (T_{1/2}), area under the blood concentration-time curve (AUC_{0-inf}), clearance (CL), and volume of distribution. Blood concentrations and computed PK parameters for MRZ will be listed and summarized by cohort (mean, geometric mean, standard deviation, coefficient of variation, minimum, maximum and number of observations). Subject population for PK will be all subjects who received at least one dose of either study drug and had at least one post-infusion sample analyzed.

Pharmacodynamics (Part 1 Phase 1 only)

Change in proteasome activities in WPB lysates and PBMC lysates, comparing pre-drug and post drug levels.

TABLE 33
Schedule of Assessments and Procedures, All Cycles

| | | | | | | | | | End of | Post Study |
|---|---|---|---|---|---|---|---|---|---|---|
| | Screen ¹ | Baseline ¹ | Cycle 1 | | | Cycle 2+ | | | Treatment ²² | Follow-up ²³ |
| | Study Day | | | | | | | | | |
| | −28 to −1 | −7 to −1 | 1 | 8 | 15 | 1 | 8 | 15 | | |
| | Window | | | | | | | | | |
| | Up to | Up to Day −8 | | ±1 | ±1 | ±1 | ±1 | ±1 | +7 | ±7 |
| Informed consent | X | | | | | | | | | |
| Medical | X | | | | | | | | | |
| Concomitant medications ¹ | X | X | X | X | X | X | X | X | X | X |
| Physical examination, height ² | | X | | | | | | | | |
| Targeted physical, weight, | | X | X | | | X | | | X | |
| [symbol] | | | | | | | | | | |
| Kamofsky Performance | | X | X | | | X | | | X | |
| Status (KPS) ³ | | | | | | | | | | |
| Neurological examination and | | X | X | | | X | | | X | |
| assessment, SARA ⁴ | | | | | | | | | | |
| Quality of life assessments | | X | | | | At the beginning of each | | | X | |
| (FACT-Cog, FACT-Br) ⁵ | | | | | | even numbered cycle | | | | |
| Toxicity evaluation ⁶ | | | X | X | X | X | X | X | | |
| Vital signs (HR, temp, BP) ⁷ | | X | X | X | X | X | X | X | X | |
| ECG ⁸ | | X | X | | X | | | | X | |
| Complete Blood Count, | | X | | X | X | X | | X | X | |
| Differential Platelets⁹ | | | | | | | | | | |
| Serum Chemistry ¹⁰ | | X | | X | X | X | | X | X | |
| PT/PTT ¹¹ | | X | X | | | X | | | X | |
| Urinalysis¹² | | X | X | | | X | | | X | |
| Marizomib infusion ¹³ | | | X | X | X | X | X | X | | |
| Bevacizumab infusion ¹⁴ | | | X | | X | X | | X | | |
| Blood PK sampling (MRZ) ¹⁵ | | | X | | X | | | | | |
| Blood PK sampling (BEV) ¹⁶ | | | X | | X | | | | | |
| Pregnancy test ¹⁷ | | X | | | | | | | X | |
| Blood Proteasome assay ¹⁸ | | | X | X | X | X | | X | X | |
| Tumor measurement ¹⁹ | X (−14 to −1) | | | | | At the end of each even | | | X | |
| | (3-day | | | | | numbered cycle | | | | |
| Tumor proteasome activity ²⁰ | X | | | | | | | | | |
| Tumor gene signature | X | | | | | | | | | |
| profiling ²¹ | | | | | | | | | | |

¹ Within 7 days of starting treatment except consent, demographics, medical history, concomitant medications, complete physical examination, radiographic/tumor assessments, and consent to acquire and test archival tumor tissue samples, which can be obtained within 28 days prior to the start of treatment.
² Height measured at baseline only. Physical Examination is a complete physical as per institutional guidelines (genitourinary examination not required unless there are related signs or symptoms) at baseline, but thereafter as directed by signs and symptoms (targeted physical examination).
³ Functional assessment using the Karnofsky Performance Status (KPS) is to be completed at baseline, at the beginning of each cycle, and at the end of treatment. See
⁴ Neurological examination, including the evaluation of coordination to be performed at baseline, at the beginning of each cycle, and at the end of treatment using the Scale for the assessment and rating of ataxia (SARA). See
⁵ Quality of life assessments using the FACT-Cog and FACT-Br are to be completed at baseline, at the beginning of each even numbered cycle (i.e., C2D1, C4D1, etc.), and at the end of treatment.. FACT forms are not validated in all languages. In cases where the patient is not fluent in a language covered by the FACT forms, these assessments will not be made.
⁶ Toxicity evaluation is an assessment of reported and observed adverse events, in the Phase 1 portion of the study, following the MRZ and BEV administrations compared to pre-dose findings. Toxicity evaluation is an assessment of reported and observed adverse events, in the Phase 2 portions (Parts 2 and 3) of the study, following the MRZ administration compared to pre-dose findings.
⁷ Vital Signs: (blood pressure, heart rate, and temperature) during the Phase 1 portion of the study in Cycle 1, Days 1 and 15: immediately before the MRZ infusion and immediately before the BEV infusion and approximately 10 (±2) minutes, 30 (±5) minutes and 1 hour (±5 minutes) following the BEV infusion. Cycle 2+, Days 1 and 15: prior to the MRZ infusion and prior to the BEV infusion and 30 (±5) minutes following each BEV infusion. In all cycles, Day 8, immediately before the MRZ infusion and 30 (±5) minutes following each MRZ infusion. During the Phase 2 portions (Parts 2 and 3) of the study, in Cycle 1, Days 1, 8, and 15: immediately before the MRZ infusion and approximately 10 (±2) minutes, 30 (±5) minutes and 1 hour (±5 minutes) following the MRZ infusion. Cycle 2+, Days 1, 8, and 15: prior to the MRZ infusion and 30 (±5) minutes following each MRZ infusion. For all portions of the study vital signs are also collected as part of the physical examination.
⁸ ECG: Eligibility ECGs must be performed within 7 days prior to Day 1. ECGs will be collected Cycle 1 only (Days 1 and 15), within 60 minutes prior to the MRZ infusion and within 5 (±1) minutes following the MRZ infusion. An End-of-Treatment ECG is to be collected. Additional ECGs should be obtained if clinically indicated.
⁹Hemoglobin (Hgb), hematocrit (Hct), red blood cell (RBC) count, white blood cell (WBC) count with differential, and platelets. Hematology tests can be performed within 72 hours of scheduled dosing except prior to Cycle 1, which can be done within 7 days prior to dosing. Should a subject experience a Grade 4 hematologic toxicity, the appropriate test will be monitored in accordance with institutional guidelines (at minimum: weekly) until Grade ≤2. The following tests should meet minimum stipulations prior to entry into Cycle 2+: Hgb ≥ 8 g/dL; platelets ≥ 75 × 10⁹/L.
¹⁰ Sodium, potassium, chloride, bicarbonate, calcium, magnesium, glucose, BUN, serum creatinine, uric acid, ALT, AST, alkaline phosphatase, total protein, albumin, and total bilirubin. Chemistry will be performed within 72 hours of scheduled dosing except prior to Cycle 1, which can be done within 7 days prior to dosing. Minimum re-treatment criterion prior to the beginning of each new cycle: creatinine ≤ 1.5 × ULN.
¹¹ Prothrombin time (PT) or International Normalized Ratio (INR) and partial thromboplastin time (PTT) may be performed more often if clinically indicated. Coagulation tests will be performed within 72 hours of scheduled dosing except prior to Cycle 1, which can be done within 7 days prior to dosing.
¹²Urinalysis: protein, blood, glucose, pH; microscopic (RBC, WBC, casts) if abnormal urinalysis. Urinalysis performed within 72 hours of scheduled dosing, except prior to Cycle 1, which can be done within 7 days prior to dosing.
¹³ Subjects are to be encouraged to maintain good oral hydration during the study (e.g., 2 liters per day, as considered appropriate by the Investigator). Part 1 MRZ infusion: injected over 10 minutes (or longer depending upon cohort). The volume of infusate will vary per subject depending on dose and BSA. In Part 1 Phase 1 subjects will receive normal saline started prior to and following the infusion administered at ~350 mL/hour, with the infusion to occur after ~350 mL have been given with a total volume of infusion to equal one liter. In Part 2 Phase 2 subjects will receive 250 mL normal saline over 30 minutes prior to the MRZ infusion. At the discretion of the Investigator, additional normal saline can be given after the MRZ infusion is complete. Part 3 Phase 2: No pre-dose hydration is required unless re-instituted after safety review by the Medical Monitor, representatives of the Sponsor, and the participating investigators. In each patient the dose will be increased if tolerated to 1.0 mg/m² after Cycle 1 and to 1.2 mg/m² after Cycle 2. Dose escalation after dose reduction is not recommended but will be allowed only with the approval of the Sponsor's Medical Monitor.
¹⁴ BEV administered as an IV infusion. First dose should be infused over 90 minutes and if tolerated, the second infusion may be given over 60 minutes, and if tolerated, subsequent infusions may be given over 30 minutes. Infusions may be interrupted or lengthened to treat or prevent infusion-related reactions. BEV is administered approximately 10 minutes after the end of the MRZ infusion. BEV is not given during Part 2 Phase 2.
¹⁵ Blood PK Sampling (MRZ) (during Part 1 Phase 1 dose escalation only): On Cycle 1 Day 1, MRZ samples will be obtained before treatment and just prior to end of infusion. On Cycle 1 Day 15, MRZ samples will be obtained before treatment, just prior to end of infusion, and 2, 5, 15, 30, 45, 60, 90, and 120 minutes after the infusion. Every effort should be made to collect samples at the prescribed times, but deviations up to 10% of the time point are allowed. Additional samples may be collected if the subject experiences a potentially drug-related SAE. Use Sponsor-provided PK kits. Process, store and ship samples per instructions in Study Reference Manual.
¹⁶ Blood PK Sampling (BEV) (during Part 1 Phase 1 dose escalation only): For BEV Cycle 1 Day 1 and 15, BEV plasma samples will be obtained before treatment and just prior to end of infusion. Use Sponsor-provided PK kits. Every effort should be made to collect samples at the prescribed times, but deviations up to 10% of the time point are allowed. Use Sponsor-provided PK kits. Process, store and ship samples per instructions in Study Reference Manual.
¹⁷ Pregnancy test (serum or urine) to be performed at Baseline, End-of-Treatment visit, and more frequently if clinically indicated.
¹⁸ Blood proteasome assay (during Part 1 Phase 1 dose escalation only): Cycle 1 Day 1 (before treatment and 1 hour post MRZ infusion), Day 8 (before treatment and 1 hour post MRZ infusion), and Day 15 (before treatment and 1 hour post MRZ infusion). Starting Cycle 2 and thereafter, Day 1 (before treatment and 1 hour post MRZ infusion) and on Day 15 (before treatment and 1 hour post MRZ infusion). A sample will be drawn at the End of Treatment visit. On Cycle 2 Day 1 (pre MRZ infusion or on Cycle 1 Day 29 if the subject does not go on to Cycle 2 or Cycle 2 is delayed. A sample will be drawn at the time that a complete response or partial response or disease progression is determined.
¹⁹ Tumor assessment: Baseline tumor assessments are to be made within 14 days (3-day time window) prior to Cycle 1 Day 1. Response should be assessed (RANO 2010) during the rest period of Cycle 2 and during the rest period of every 2 cycles thereafter (±7 days). If a subject is determined to have an overall disease response of CR or PR, then disease assessments should be repeated approximately 4 (±2 days) weeks later to confirm the response. If tumor assessments have not been performed in the 4 weeks prior to the End-of Treatment Visit, then tumor assessments are to be done at the End-of Treatment Visit. If a patient has a standard of care tumor assessment done prior to giving Informed Consent, but within the 14 day (3-day window), that is available to the investigator, then that tumor assessment can serve as a baseline and another screening MRI is not required.
²⁰ For subjects with flash-frozen, GBM tumor tissue, assessment of pre-treatment proteasome activity levels will be performed.
²¹ For subjects with archived blocks of GBM tumor, genomic analysis and transcriptional profiling will be conducted. A blood sample will also be collected prior to C1D1 dosing in these subjects so comparisons can be made between germ line and tumor mutations.
²² Subjects with drug-related AEs of Grade ≥2 observed at the End-of-Treatment assessment should be followed-up at least monthly until the AE has resolved to Grade 1, the event is believed to be chronic or subject receives other anti-cancer therapy.
²³ Post Study Follow-up visits may be made in person or other means of communication. Purpose of the follow up, which should occur every 3 months (±7 days), is to determine survival and the start of first new anti-GBM systemic treatment and its outcome. [symbol]
indicates data missing or illegible when filed

1. STUDY OBJECTIVES
1.1. Primary Objective

### Part 1 Phase 1

The primary objective of the study is To determine the maximum tolerated dose (MTh) or maximum administered dose (MAD) and recommended Phase 2 dose (RP2D) of the combination of marizomib (MRZ)+bevacizumab (BEV) with MRZ as a once weekly dose for 3 weeks of a 28-day cycle and a fixed dose and schedule of BEV (10 mg/kg administered on Days 1 and 15) in subjects with WHO Grade 4 malignant glioma (G4 MG), who have not previously been treated with either an anti-angiogenic agent including, but not limited to, BEV or a proteasome inhibitor including, but not limited to, MRZ.

### Part 2 Phase 2

To assess the activity of a once weekly dose for 3 weeks of a 28-day cycle of MRZ in subjects with progressive or recurrent G4 MG, who have not previously been treated with either an anti-angiogenic agent or a proteasome inhibitor.

### Part 3 Phase 2

To assess the activity of the combination of once weekly MRZ dosing for 3 weeks (allowing for intra-patient dose escalation) and every other week dosing of BEV at 10 mg/kg in 28-day cycle in subjects with progressive or recurrent G4 MG, who have not previously been treated with either an anti-angiogenic agent or a proteasome inhibitor.
1.2. Secondary Objectives

### Part 1 Phase 1

The secondary objectives of the study are:
To evaluate the safety of the combination of MRZ+BEV in the subject population.
To evaluate activity of the combination of MRZ+BEV in the subject population:
   Radiographic Response Rate
   Progression-free Survival (PFS)
   Overall Survival (OS)
To evaluate the pharmacokinetics (PK) of MRZ and BEV when administered in combination in the subject population.
To assess the whole blood proteasome pharmacodynamic (PD) activity of the combination of MRZ+BEV in the subject population.

### Part 2 Phase 2

To evaluate the safety of single agent MRZ in the subject population.

### Part 3 Phase 2

To evaluate the safety of combination of MRZ and BEV with intrapatient dose escalation and BEV at a fixed dose in the subject population.
1.3. Exploratory Objectives

### Part 1 Phase 1, Part 2 Phase 2, and Part 3 Phase 2

The exploratory objectives of the study are
To evaluate baseline tumor proteasome activity, gene signature, and transcriptional profiling and correlation with activity using pre-study, archived tissue samples.
To evaluate neurological coordination assessment using the Scale for the Assessment and Rating for Ataxia (SARA).
To evaluate Quality of Life Assessments using the Functional Assessment of Cancer Therapy (FACT) questionnaires:
   FACT-Cognitive Function (FACT-Cog)
   FACT-Brain (FACT-Br)
2. STUDY ENDPOINTS
2.1. Primary Endpoint(s)

### Part 1 Phase 1

Maximum tolerated dose (MTD) or Maximum Administered Dose (MAD)
Recommended Phase 2 Dose (RP2D)

### Part 2 Phase 2

Best response

### Part 3 Phase 2

Overall survival (OS)
2.2. Secondary Endpoint(s)

### Part 1 Phase 1 and Part 2 Phase 2

### Safety

Type, incidence and severity of adverse events (AEs)
Type, incidence and severity of serious adverse events (SAEs)
Type, incidence and severity of dose-limiting toxicities (DLTs)

### Activity

Radiographic overall response rate (ORR)
Progression-free survival (PFS)
Overall survival (OS)

### Part 3 Phase 2

### Safety

Type, incidence and severity of adverse events (AEs)
Type, incidence and severity of serious adverse events (SAEs)
Type, incidence and severity of dose-limiting adverse events (DLAEs)

### Activity

Radiographic overall response rate (ORR)
Progression-free survival (PFS)
   Pharmacokinetics for MRZ (Part 1 Phase 1 only)
Maximum observed blood drug concentration (Cₘₐₓ)
Time of maximum blood concentration (tₘₐₓ)
Elimination half-life (t_{1/2})
Area under the blood concentration-time curve (AUC₀₋₄, AUC_{0-inf})
Clearance (CL)
Volume of distribution (Vd)
   Pharmacokinetics for BEV (Part 1 Phase 1 only)
Peak and trough levels in plasma
   Pharmacodynamic Assessment (Part 1 Phase 1 only)
Change in proteasome activities in packed whole blood (PWB)
2.3. Exploratory Endpoint(s)

### Part 1 Phase 1, Part 2 Phase 2 and Part 3 Phase 2

To evaluate baseline tumor proteasome activity, gene signature and transcriptional profiling using pre-study, archived tissue samples.
To evaluate neurological coordination assessment using the Scale for the Assessment and Rating for Ataxia (SARA).
To evaluate Quality of Life Assessments using Functional Assessment of Cancer Therapy (FACT) questionnaires:
   FACT-Cognitive Function (FACT-Cog)
   FACT-Brain (FACT-Br)
3. OVERALL STUDY DESIGN
3.1. Study Design

This is a Phase 1, multicenter, open-label, 3+3, dose-escalation study in subjects with G4 MG who are in first or second relapse and who have not previously received any BEV or other anti-angiogenic agent, including: sorafenib, sunitinib, axitinib, pazopanib, everolimus, or cilengitide or MRZ or any other proteasome inhibitor, including bortezomib (BTZ), carfilzomib (CFZ), or ixazomib (IXZ). Three to 6 evaluable subjects per cohort will be enrolled: up to 24 subjects to determine the MTD/MAD (Part 1 dose-escalation) and an additional 12 or more subjects to confirm the MTD/MAD (Part 2 MTD/MAD expansion) to a total of up to 36 subjects and assess preliminary activity. Subjects may not be enrolled in more than 1 cohort and there will be no intra-subject dose escalation.

The Phase 1 portion will be followed by a Phase 2 portion of the trial of single agent MRZ administered as a 10-minute infusion at a dose of 0.8 mg/m² (the MAD) every week for 3 weeks in 28-day cycles. This portion of the trial will be conducted as a 2-stage sequential design of up to 30 response-evaluable patients.

The Part 2 Phase 2 portion of the trial will be followed by Part 3 Phase 2 of combination MRZ and BEV. MRZ will be administered as a 10-minute infusion every week for 3 weeks in 28-day cycles at a starting dose of 0.8 mg/m² (the RP2D from Phase 1). After 1 cycle without a DLAE, the dose will be incremented to 1.0 mg/m² and then, if the dose is tolerated, to 1.2 mg/m² in Cycle 2 and thereafter. BEV will be administered every 2 weeks (Days 1 and 15 of each 28-day cycle) at a fixed dose of 10 mg/kg. This portion of the trial will be conducted with 40 eligible patients.
3.2. Study Design Rationale

### Part 1 Phase 1

The study is a classical 3+3 design that is often used in Phase 1 cancer studies. Standard evaluations for safety and activity are employed. The schema is provided in FIG. 19.

### Part 2 Phase 2

The study is a modified 2-stage design (Green and Dahlberg 1992). Standard evaluations for safety and activity are employed. Fifteen response-evaluable patients will be entered in the first stage. If no objective responses are observed, the trial will be terminated. If 1 or more responses are observed, then the second stage will be implemented with an additional 15 response-evaluable patients treated. If at least 5 responses are observed, MRZ will be considered active as a single agent.

### Part 3 Phase 2

The study is designed to determine the OS in patients treated with intrapatient dose escalation of MRZ with a constant dose of BEV.
3.3. Study Duration

Subjects may continue on study treatment until disease progression, unacceptable toxicity, withdrawal of consent, or termination of the study. Once discontinued from the study treatment, subjects will enter a long-term follow-up period (Post Study Follow-Up) for documentation of survival and the start of first new anti-GBM therapy and its outcome. Post Study Follow-up will occur every 3 months (±7 days) after the 28-day post-treatment discontinuation visit (End-of-Treatment visit).
3.4. End of Trial

The End of Trial is defined as the date of receipt of the last data point from the last remaining subject that is required for primary, secondary and/or exploratory analysis.

### Procedures

Study visits and procedures will be performed as outlined in Table 33.

The study will consist of Screening, Baseline, Treatment, and Follow-up periods. Except where otherwise stated, the procedures apply to both Phase 1 and Phase 2 portions of the study.

### Screening

Screening procedures may not be done prior to the signing and dating of the Informed Consent Form (ICF). However, the results of tumor assessments done as part of standard of care that are within the 14-day (3-day window) screening period do not have to be repeated if they were done at the participating site. However, if results are not available, then tumor assessments are to be conducted within 14 days prior to Cycle 1 Day 1. The screening period for assessments that include medical history (including demographics and cancer history), prior medications and procedures may not exceed a 28 days (with a 3-day window for scheduling conflicts) window prior to start of study treatment (Cycle 1 Day 1) for assessments that include medical history including demographics and cancer history, prior medications and procedures.

### Baseline

Physical examination including height, weight, and vital signs, Karnofsky Performance Status (KPS) scale, ECGs, laboratory tests including hematology, coagulation, chemistry, urinalysis; and, as appropriate, pregnancy tests are to be done within 7 days (with a 2-day window for scheduling conflicts)prior to Cycle 1 Day 1. Neurological coordination assessment using the Scale for the Assessment and Rating of Ataxia (SARA) and quality of life assessment using the FACT-Cog and FACT-Br.

### Treatment

Safety tests and procedures will be performed according to the Schedule of Assessments and Procedures (Table 33). PK and PD samples will be obtained prior to the start of treatment and then after dosing at selected time points for Part 1 Phase 1. Assessments will include MRI scans at the end of every even numbered cycle (±7 days) using RANO 2010 criteria for assessment. Patients in the Part 1 Phase 2 and Part 3 Phase 2 portions of the study may continue treatment with MRZ for 1 or 2 cycles after an MRI indicates progression if according to the investigator's judgement this is in the best interest of the patient and/or if the investigator interprets that the MRI indicates possible pseudoprogression, and there is no significant clinical deterioration of the patient.

Functional status using the KPS will be conducted in both Phases. Neurological coordination assessment using the SARA and quality of life assessment using the FACT-Cog and FACT-Br will be assessed regularly.

Subjects may continue on study treatment until disease progression, unacceptable toxicity, withdrawal of consent, or termination of the study.

### End-of-Treatment Visit

An End-of-Treatment Visit should occur when a subject discontinues study treatment 28 (+7) days after the last dose of MRZ or BEV, whichever is later in the Part 1 Phase 1 and Part 3 Phase 2 portions and after the last dose of MRZ in the Part 2 Phase 2 portion of the study. Tests are primarily to ensure there are no late occurring AEs and that AEs have resolved or have stabilized. Additional follow-up visits may be conducted to follow ongoing AEs that are resolving. If a subject cannot or will not make this visit, attempts to gather information on the status of AEs should be made by telephone or other means.

### Post Study Follow-up

All subjects will be followed for survival during the follow-up period for as long as they are alive. Post Study follow-up will occur every 3 months (±7 days) after the End-of-Treatment Visit. During long-term follow-up, the following information will be collected: survival, first subsequent anti-GBM systemic regimens, and treatment outcomes.

### Activity Assessments

Tumor response, including progressive disease, will be assessed with MRI at the end of every 2 cycles of therapy according to the RANO criteria, including:
Radiographic Overall Response Rate
Progression-free Survival (PFS)
Overall Survival (OS)

Confirmation of response at 4 weeks (±2 days) after the response is to be performed.

If the Investigator believes that an MRI indicating tumor progression by RANO criteria may reflect pseudoprogression, and there is no significant clinical deterioration of the patient, the patient may be continued for 1 or 2 additional cycles (at the discretion of the Investigator based on the patient's clinical condition) before another MRI assessment is conducted. If a patient is taken off study for progressive disease by imaging and subsequent biopsy or surgical resection shows no evidence of disease, the patient will be counted as a responder. In this case, the patient may return to the trial for additional treatment with MRZ, using a post-procedure MRI as the new baseline.

Pharmacokinetic (PK) Assessments: MRZ (Part 1 Phase 1 only)

Blood should be drawn from the contralateral arm to the infusion site and using an indwelling catheter to avoid multiple needle sticks is recommended. Sample collection time should be recorded on the tube label and Case Report Form (CRF) as day: hour: minute. Nominal time of blood collection are given as “time points”; it is critical that an accurately collected actual time of the sample is written on the CRF and on the blood tubes (date entered as dd:mm:yyyy; time entered using a 24-hour clock, hh:mm). For MRZ samples will be obtained before treatment and just prior to end of infusion on Cycle 1 Day 1. On Cycle 1 Day 15, MRZ blood samples will be obtained before treatment, just prior to end of infusion, and 2, 5, 15, 30, 45, 60, 90, and 120 minutes after the infusion. Every effort should be made to collect samples at the prescribed times, but deviations up to 10% of the time point are allowed. Additional samples may be collected if the subject experiences a potentially drug-related SAE. After blood collection, neutralizing solution must be added. Use Sponsor-provided PK kits. Process, store and ship samples per instructions in Study Reference Manual.

PK parameters that will be determined include:
Maximum observed blood drug concentration (Cₘₐₓ)
Time of maximum blood concentration (tₘₐₓ)
Elimination half-life (t_{1/2})
Area under the blood concentration-time curve (AUC₀₋₄, AUC_{0-inf})
Clearance (CL)
Volume of distribution (Vd)
   PK Assessments: BEV (Part 1 Phase 1 only)

For BEV pre-dose and immediately prior to EOI, plasma samples will be taken on Days 1 and 15 to assess BEV peak and trough levels. Process, store and ship samples per instructions in Study Reference Manual. PK assessment of BEV is not done in Phase 2.

Pharmacodynamic (PD) Assessments (Part 1 Phase 1 only)
Change in proteasome activities (whole blood lysates and PBMC lysates), comparing pre-drug and post drug levels

The laboratory correlates include assessment of the percentage inhibition of proteasome function (evaluated by measurement of CT-L, T-L and C-L activity in blood isolates such as whole blood and PBMC lysates.

Process, store and ship samples per instructions in Study Reference Manual.

### Tumor Proteasome Activity

For subjects with flash frozen GBM tumor tissue, proteasome activity will be determined by qualified assay. Instructions on shipping these samples will be provided in the Study Reference Manual.

### Gene Profiling

Prior to Amendment 2, gene signature profiling was to be performed using the Decision Dx-GBM test from Castle Bioscience to determine the molecular signature for a GBM tumor. This assay was not conducted on any patient samples, and no future testing by this method is planned. After Amendment 2 is approved, for subjects with archived blocks of GBM tumor, a proteasome based gene expression analysis will be performed. This analysis will focus on quantitation of the β1, β2, and β5 catalytic subunits of the proteasome to determine whether gene expression correlates to enzymatic activity measured in frozen tumor tissue. If a relationship is observed between enzymatic activity and gene expression, this may enable a correlation between MRZ clinical response and proteasome subunit expression in archival tissue. These studies aim to determine whether a predictive biomarker for MRZ-responsive patients can be identified. If we are unable to observe a correlation between enzymatic activity and catalytic subunit gene expression levels, we may undertake a wider genetic profiling study to determine whether a gene signature predictive of MRZ response can be identified. Instructions on shipping these samples will be provided in the Study Reference Manual.

### Karnofsky Performance Status

The Karnofsky Performance Status (KPS) allows patients to be classified as to their functional impairment. This can be used to measure changes in a patient's ability to function. The Karnofsky Performance Status scores range from 0 to 100. A higher score signifies the patient is better able to carry out daily activities.

### Scale for the Assessment and Rating of Ataxia

The Scale for the Assessment and Rating of Ataxia (SARA) is a clinical scale that is based on a semiquantitative assessment of cerebellar ataxia on an impairment level. It has eight items with total scores ranging from 0 (no ataxia) to 40 (most severe ataxia). Scores for the eight items range as follows: no ataxia, 1: gait (0-8 points), 2: stance (0-6 points), 3: sitting (0-4 points), 4: speech disturbance (0-6 points), 5: finger chase (0-4 points), 6: nose-finger test (0-4 points), 7: fast alternating hand movement (0-4 points), 8: heel-shin slide (0-4 points), and 40: severe ataxia. For motor activities of the four extremities (items 5-8), assessments are performed bilaterally, and the mean values are used to obtain the total score.

### Quality of Life Assessments

FACT-Cog

The Functional Assessment of Cancer Therapy-Cognitive Function (FACT-Cog) is a 37-item validated subjective neuropsychological instrument designed to evaluate cancer subjects' perceived cognitive deterioration on their quality of life. A 6.9 to 10.6 points reduction of the FACT-Cog score corresponds to the smallest clinically-relevant perceived cognitive deterioration. These estimates are important as they can facilitate the interpretation of subjects'-reported cognitive changes and sample size estimation.
FACT-Br

The Functional Assessment of Cancer Therapy-Brain (FACT-Br) is a commonly used instrument measuring general quality of life (QOL) that reflects symptoms or problems associated with brain malignancies across 5 scales. The measure yields information about total QOL, as well as information about the dimensions of physical well-being, social/family well-being, emotional well-being, functional well-being, and disease specific concerns. The FACT-Br is written at the 4th grade reading level, and subjects can fill it out in 5-10 minutes. The self-report can be completed by the subject with little or no assistance in subjects who are not neurologically incapacitated. Subjects rate all 5 items using a 5-point Likert scale ranging from 0 (not at all) to 4 (very much). Overall, higher ratings suggest higher QOL. Items are totaled to produce the following subscales, along with an overall QOL score: physical well-being (7 items); social/family well-being (7 items); emotional well-being (6 items); functional well-being (7 items); and concerns relevant to subjects with brain tumors (23 items).

### Study Population

The study population includes subjects with G4 MG (including glioblastoma and gliosarcoma) who are in first or second relapse and who have not previously received any BEV or other anti-angiogenic agent, including sorafenib, sunitinib, axitinib, pazopanib, everolimus, or cilengitide or MRZ or any other proteasome inhibitor, including BTZ, CFZ, or IXZ.
3.5. Number of Subjects and Sites

Thirty-six subjects were enrolled in the study at multiple sites in Part 1 Phase 1 and 30 response-evaluable patients will be enrolled in Part 2 Phase 2. Forty response evaluable patients in Part 3 Phase 2 will be enrolled as per this Amendment 3.
3.6. Inclusion Criteria

Subjects must satisfy the following criteria to be enrolled in the study. These criteria apply to both the Phase 1 portion including the expansion cohort and both the Part 2 and Part 3 Phase 2 portions.
1. Understand and voluntarily sign and date an informed consent document prior to any study related assessments/procedures are conducted.
2. Males and Females 18 years of age at the time of signing the informed consent document.
3. All subjects must have histologic evidence of G4 MG (including glioblastoma and gliosarcoma) and radiographic evidence of recurrence or disease progression (defined as either a greater than 25% increase in the largest bidimensional product of enhancement, a new enhancing lesion, or significant increase in T2 FLAIR). Subjects must have at least 1 measurable lesion by RANO criteria (≥10 mm in 2 perpendicular diameters).
4. Subjects must have previously completed standard radiation therapy and been exposed to temozolomide. Patients must be in first or second relapse.
5. Subjects with archival tumor tissue suitable for proteasome activity and genetic testing must give permission to access and test the tissue; subjects without archival tumor tissue are eligible.
6. No prior treatment with MRZ or any other proteasome inhibitors, including BTZ, CFZ, and IXZ or BEV or any other anti-angiogenic agents, including sorafenib, sunitinib, axitinib, pazopanib, everolimus or cilengitide.
7. No investigational agent within 4 weeks prior to first dose of study drug.
8. At least 4 weeks from surgical resection and 12 weeks from end of radiotherapy prior to enrollment in this study, unless relapse is confirmed by tumor biopsy, or new lesion outside of radiation field, or if there are two MRIs confirming progressive disease that are 8 weeks apart.
9. Subjects with a history of seizures must be on a stable dose of anti-epileptic drugs (AEDs) and without seizures for 14 days prior to enrollment in patients enrolled prior to Amendment 2. Subjects enrolled after Amendment 2 is approved with a history of seizures must be on a stable dose of anti-epileptic drugs (AEDs) for 7 days prior to enrollment.
10. All AEs resulting from prior chemotherapy, surgery, or radiotherapy, must have resolved to US National Cancer Institute Common Terminology Criteria for Adverse Events Version 4.03 (NCI-CTCAE v. 4.03) Grade (except for laboratory parameters outlined below).
11. Laboratory results within 7 days prior to MRZ administration (transfusions and/or growth factor support may not be used to meet this criteria):
   Platelet count≥100×10⁹/L.
   Hemoglobin≥9 g/dL.
   Absolute neutrophil count (ANC)≥1.5×10⁹1.
   Serum bilirubin≤1.5×upper limit of normal (ULN) or ≤3×ULN if Gilbert's disease is documented.
   Aspartate transaminase (AST)≤2.5 ULN.
   Alanine transaminase (ALT)≤2.5 ULN.
   Serum creatinine≤1.5×ULN.
   Urine protein: creatinine ratio≤1.0
12. Karnofsky Performance Status (KPS) score ≥70%.
13. For women of child-bearing potential and for men with partners of child-bearing potential, subject must agree to take contraceptive measures for duration of treatment and for 3 months after the last dose of MRZ or 6 months after the last dose of BEV, whichever is longer.
14. Willing and able to adhere to the study visit schedule and other protocol requirements.
3.7. Exclusion Criteria

The presence of any of the following will exclude a subject from enrollment. Co-medication that may interfere with study results, e.g., immuno-suppressive agents other than corticosteroids. (Steroid therapy for control of cerebral edema is allowed at the discretion of the Investigator. Subjects should be on a stable dose of steroids for at least 1 week prior to first dose of MRZ.)
1. Evidence of CNS hemorrhage on baseline MRI or CT scan (except for post-surgical, asymptomatic Grade 1 hemorrhage that has been stable for at least 3 months for subjects enrolled prior to Amendment 2 and for at least 4 weeks in subjects enrolled after Amendment 2 is approved).
2. History of thrombotic or hemorrhagic stroke or myocardial infarction within 6 months.
3. Chemotherapy administered within 4 weeks (except 6 weeks for nitrosoureas, 12 weeks for nitrosourea wafer, and 1 week from metronomic chemotherapy, like daily temozolomide and etoposide) prior to Day 1 of study treatment, unless the subject has recovered from all expected toxicities from the chemotherapy.
4. Pregnancy or breast feeding.
5. Uncontrolled intercurrent illness including, but not limited to, ongoing or active infection requiring IV antibiotics & psychiatric illness/social situations that would limit compliance with study requirements, or disorders associated with significant immunocompromised state.
6. Known previous/current malignancy requiring treatment within ≤3 years except for cervical carcinoma in situ, basal cell carcinoma, and superficial bladder carcinoma.
7. Any comorbid condition that confounds the ability to interpret data from the study as judged by the Investigator or Medical Monitor.
   BEV-Specific Concerns (All Parts) (Note: These Exclusion Criteria Apply to the Part 2 Phase 2 Portion of the Study Even Though BEV is not Administered so that the Patient Populations Among Part 1, Part 2, and Part 3 are Similar):
8. Any prior history of hypertensive crisis or hypertensive encephalopathy.
9. Systolic blood pressure (BP)>150 mmHg or diastolic BP>100 mmHg.
10. Unstable angina.
11. New York Heart Association Grade≥II congestive heart failure.
12. History of myocardial infarction within 6 months.
13. Subjects with mean QTcF interval>500 ms.
14. Clinically significant peripheral vascular disease.
15. Evidence of bleeding diathesis or coagulopathy as documented by an elevated (≥1.5×ULN) prothrombin time (PT), partial thromboplastin time (PTT), or bleeding time. The use of full-dose oral or parenteral anticoagulants is permitted as long as the PT or aPTT is within therapeutic limits (according to the medical standard of the enrolling institution) and the subject has been on a stable dose of anticoagulants for at least 2 weeks prior to the first study treatment.
16. Major surgical procedure, open biopsy, or significant traumatic injury within 28 days prior to Day 1 or anticipation of need for major surgical procedure during course of the study.
17. Minor surgical procedures, fine needle aspirations or core biopsies within 7 days prior to Day 1.
18. History of abdominal fistula, GI perforation, or intra-abdominal abscess within 6 months prior to Day 1.
19. Serious, non-healing wound, ulcer, or bone fracture requiring surgical intervention.

### Description of Study Treatments

3.8. Treatment Administration and Schedule

### 7.2.1. Marizomib Drug Product

The lyophilized drug product contains 2 mg API and 60 mg sucrose bulk excipient. Cartons contain one vial of lyophile together with a Diluent vial containing 55% propylene glycol, 5% ethanol, and 40% citrate buffer pH 5 (20 mL fill; 10 mL intended for use). The lyophile drug product reconstituted with 10 mL diluent results in a dosing solution comprised of 55% propylene glycol, 40% citrate buffer and 5% ethanol, with 6 mg/mL sucrose as a pharmaceutical excipient. The drug is delivered at 0.2 mg/mL at a final dosing solution of pH ˜6. A dose of 0.7 mg/m² will result in approximately 7 mL of infusate.
3.8.1. Administration of MRZ in Part 1 Phase 1

MRZ will be administered IV over 10 minutes or longer depending upon cohort (refer to Directions for Use regarding directions for administration time). Volume of administration will vary based on assigned dose (see Table 34) and subject body surface area (BSA). To mitigate the possibility of renal dysfunction, subjects will receive normal saline administered at 350 mL/hour for 1 hour before and for 2 hours after the MRZ infusion. The MRZ infusion will be started after approximately 350 mL of saline have been given over 1 hour. After the MRZ infusion has been completed, approximately 700 mL of saline will be given over 2 hours, for a total volume of saline infusion equal to approximately 1 L.

Subjects should maintain good oral hydration during the study (e.g., 2 L/day). The volume and duration of hydration may be reduced at the discretion of the Investigator, especially for subjects with low body weight or with conditions sensitive to fluid overload.

Subjects must not drive a vehicle or operate heavy machinery while on this study.

The lyophilized drug product contains 2 mg API and 60 mg sucrose bulk excipient. Cartons contain one vial of lyophile together with a Diluent vial containing 55% propylene glycol, 5% ethanol, and 40% citrate buffer pH 5 (20 mL fill; 10 mL intended for use). The lyophile drug product reconstituted with 10 mL diluent results in a dosing solution comprised of 55% propylene glycol, 40% citrate buffer and 5% ethanol, with 6 mg/mL sucrose as a pharmaceutical excipient. The drug is delivered at 0.2 mg/mL at a final dosing solution of pH ˜6. A dose of 0.7 mg/m² will result in approximately 7 mL of infusate.
3.8.2. Administration of BEV in Part 1 Phase 1 and Part 3 Phase 2

BEV will be administered as an IV infusion (90 minutes 1^{st} dose, and if tolerated 60 minutes 2^{nd} dose and 30 minutes on subsequent doses if tolerated) as described in the current package insert. MRZ will be administered prior to BEV when co-administered on the same day.
3.8.3. Administration of MRZ in Part 2 Phase 2

MRZ (0.8 mg/m²) will be administered IV over 10 minutes (refer to Directions for Use). To mitigate the possibility of renal dysfunction, subjects will receive normal saline administered at 250 mL over ˜30 minutes before the MRZ infusion. The lyophilized drug product is the same as used in Phase 1.

Subjects should maintain good oral hydration during the study (e.g., 2 L/day). The volume and duration of hydration may be reduced at the discretion of the Investigator, especially for subjects with low body weight or with conditions sensitive to fluid overload.

Subjects must not drive a vehicle or operate heavy machinery while on this study.
3.8.4. Administration of MRZ in Part 3 Phase 2

MRZ will be administered IV over 10 minutes (refer to Directions for Use). The lyophilized drug product is the same as used in Part 1 Phase 1 and Part 2 Phase 2.

Starting dose in each patient will be 0.8 mg/m². Intrapatient dose escalation will be used. Patients who tolerate the dose in Cycle 1 will have the dose increased by 0.2 mg/m² to 1.0 mg/m² for Cycle 2 (an increase of 25%) and if the Cycle 2 dose is tolerated, to 1.2 mg/m² (an increase of 20%) for Cycle 3 and subsequent cycles.

Because renal damage has not been a safety issue with the shortened hydration schedule used in Part 2 Phase 2, intravenous hydration prior to MRZ dosing will not be used. Subjects should be encouraged to maintain good oral hydration during the study (e.g., 2 L/day). Should renal function become a safety issue, predose hydration will be reinstated if thought necessary by the medical monitor and study team and the participating investigators.

Subjects must not drive a vehicle or operate heavy machinery while on this study.
3.8.5. Dose Schedules

### Part 1 Phase 1

All subjects will receive MRZ+BEV as follows:

### IV Marizomib (MRZ)

MRZ will be administered as a 10-minute IV infusion on Days 1, 8, and 15 of every 28-day cycle. Infusion durations may be lengthened to ameliorate toxicity for individual subjects or for cohorts with agreement between the Investigators and the Sponsor. For dosing details, see Table 34.
Minimum re-treatment criterion prior to the beginning of each new cycle: creatinine≤1.5×ULN, Hgb≥8 g/dL, platelets≥75×10⁹/L.

### IV BEV

BEV will be administered as an IV infusion (90 minutes 1st dose, 60 minutes 2nd dose and 30 minutes afterward) at a dose of 10 mg/kg on Days 1 and 15. BEV will be administered approximately 10 minutes after the end of the MRZ infusion when co-administered on the same day.

In the case of dosing delay, BEV should always be given on the day that MRZ is administered. If BEV is discontinued for AEs, the subject may continue on MRZ alone. If MRZ is discontinued, then the subject will be discontinued from the trial. If MRZ is delayed, BEV should also be delayed. Both drugs will be discontinued once disease progression is documented.

MRZ dosing will begin at 0.55 mg/m² once weekly (Cohort 1). Additional dose cohorts are planned as shown in Table 34.

TABLE 34
Dose Cohorts for MRZ + BEV Combination

| | IV MRZ | IV BEV |
|---|---|---|
| | Day 1, 8, and 15 of | Day 1 and 15 of |
| Cohort | Each 28-Day Cycle | Each 28-Day Cycle |
| −2 | 0.3 mg/m² | 10 mg/kg |
| −1 | 0.4 mg/m² | 10 mg/kg |
| 1 | 0.55 mg/m² | 10 mg/kg |
| 2 | 0.7 mg/m² | 10 mg/kg |
| 3 | 0.8 mg/m² | 10 mg/kg |
| 4 | Additional cohorts with extended | 10 mg/kg |
| | infusion duration if required | |

### Part 1 Phase 1 Expansion Cohort and Part 2 Phase 2

### IV Marizomib (MRZ)

MRZ (0.8 mg/m²) will be administered as a 10-minute IV infusion on Days 1, 8, and 15 of every 28-day cycle. Infusion durations may be lengthened to ameliorate toxicity for individual subjects with agreement between the Investigator and the Sponsor.

Detailed instructions for MRZ dose modifications and actions are provided in Table 35.

TABLE 35
Marizomib Dose Modification Guidelines for
Part 1 Phase 1 Expansion and Part 2 Phase 2

| Toxicity | MRZ Dose Modification & Action |
|---|---|
| Grade 2 Central | Consider holding MRZ until toxicity resolves. |
| Nervous System | When toxicity resolves, consider reinitiating |
| Disorders | with reduced dose of MRZ (to be determined in |
| | discussion with the Medical Monitor, but at |
| | least a decrease of 0.1 mg/m²). |
| Grade 3 Nervous | Hold MRZ until toxicity resolves. When |
| System Disorder | toxicity resolves, reinitiate with reduced dose of |
| AEs | MRZ (to be determined in discussion with the |
| | Medical Monitor, but at least a decrease of 0.1 |
| | mg/m² at start of next cycle). |
| Other Grade 3 | Hold MRZ until toxicity resolves. When |
| MRZ-related | toxicity resolves, reinitiate with reduced dose of |
| AEs | MRZ (to be determined in discussion with the |
| | Medical Monitor, but at least a decrease of 0.1 |
| | mg/m² at start of next cycle). |
| Grade 4 Hematologic | Hold MRZ until toxicity resolves. When |
| MRZ-related AEs | toxicity resolves, reinitiate with reduced dose of |
| | MRZ (to be determined in discussion with the |
| | Medical Monitor, but at least a decrease of 0.1 |
| | mg/m² at start of next cycle). |
| Nonhematological | Permanently discontinue all study treatment. |
| Grade 4 MRZ-related | |
| AEs | |

This table was added with Amendment 2 and applies to patients entered under Amendment 2 whether in the Phase 1 Dose Expansion or Phase 2 portions of the study.

In addition to the guidelines in Table 35 if a subject has a drug-related event that requires a 14-day delay in therapy, then MRZ dose reduction is appropriate. If recovery from toxicities is prolonged beyond 14 days, then the dose of MRZ will be decreased by 0.1 mg/m² when dosing resumes. After MRZ dose interruption, reassessment of safety laboratory tests is required prior to resuming MRZ treatment. Prior to initiation of subsequent cycles, results for the following tests must meet study entry criteria: liver functions tests (LFTs), serum creatinine, and complete blood count.

The minimum permitted dose level for MRZ is 0.5 mg/m². If toxicity recurs at the minimum permitted dose of MRZ, all study treatment should be discontinued. Dose re-escalation is not permitted for MRZ.

### Part 3 Phase 2

### IV Marizomib

MRZ will be administered as a 10-minute IV infusion on Days 1, 8, and 15 of every 28-day cycle.
Starting dose for each patient will be 0.8 mg/m².
Doses will be rounded to the nearest tenth of a mg.
Assuming the patient tolerates the dose in Cycle 1, the dose will be increased to 1.0 mg/m² for Cycle 2, and if that dose is tolerated to 1.2 mg/m² in Cycle 3 and beyond.
DLAEs are MRZ-related AEs 1) related to disturbances in the cerebellum (i.e., ataxia, dizziness, dysarthria, fall, gait disturbances) plus hallucinations of any grade or 2) Grade≥2 other AEs.
Doses will be dose delayed and/or dose reduced or discontinued for DLAEs related to MRZ as described in Table 36.

TABLE 36
Marizomib Dose Modification Guidelines in Part 3 Phase 2

| Toxicity | MRZ Dose Modification & Action |
|---|---|
| Grade 2 Central | Monitor the toxicity. Medically treat the toxicity |
| Nervous System | if treatment is available. If treatment is successful, |
| Adverse Events | maintain the dose. If the patient cannot tolerate |
| | the toxicity consider dose delay and/or dose |
| | reduction. Discuss the case with the Medical |
| | Monitor. |
| Grade 3 Central | Medically treat the toxicity if treatment is |
| Nervous System | available. If treatment is successful, maintain the |
| Adverse Events | dose. If treatment is unsuccessful, hold MRZ until |
| | toxicity resolves to Grade 1 or less. When toxicity |
| | resolves, consider reinitiating with reduced dose |
| | of MRZ to be determined in discussion with the |
| | Medical Monitor, but at least a decrease of 0.1 |
| | mg/m² at start of next dose. |
| Other Grade 3 | Medically treat the toxicity if treatment is |
| MRZ-related | available. If treatment is successful, maintain the |
| Adverse Events | dose. If treatment is not successful, hold MRZ |
| | until toxicity resolves to Grade 1 or less. When |
| | toxicity resolves, reinitiate with reduced dose of |
| | MRZ to be determined in discussion with the |
| | Medical Monitor, but at least a decrease of |
| | 0.1 mg/m² at start of next dose. |
| Grade 4 Hematologic | Medically treat the toxicity if treatment is |
| MRZ-related | available. Hold MRZ until toxicity resolves to |
| Adverse Events | Grade 1 or less. When toxicity resolves, consider |
| | reinitiating with reduced dose of MRZ to be |
| | determined in discussion with the Medical |
| | Monitor, but at least a decrease of 0.1 mg/m² at |
| | start of next dose). |
| Nonhematological | Permanently discontinue all study treatment. |
| Grade 4 MRZ-related | |
| Adverse Events | |

In addition to the guidelines in Table 36, if a subject has a MRZ-related event that requires a 14-day delay in therapy (calculated from the scheduled date of the next dose), then MRZ dose reduction is appropriate. If recovery from MRZ-related AEs is prolonged beyond 14 days, then the dose of MRZ will be decreased by 0.1 mg/m² when dosing resumes unless an alternative plan is approved by the Sponsor's Medical Monitor.
Dose re-escalation is not permitted for MRZ unless approved by the Sponsor's Medical Monitor.
All dose increases require approval of the Sponsor's Medical Monitor.

### IV BEV

BEV will be administered as an IV infusion (90 minutes 1st dose, 60 minutes 2nd dose and 30 minutes afterward) at a dose of 10 mg/kg on Days 1 and 15. BEV will be administered approximately 10 minutes after the end of the MRZ infusion when co-administered on the same day.

In the case of dosing delay, BEV should always be given on the day that MRZ is administered. If BEV is discontinued for AEs, the subject may continue on MRZ alone. If MRZ is discontinued, then the subject will be discontinued from the trial. If MRZ is delayed, BEV should also be delayed. Both drugs will be discontinued once disease progression is documented.

There are no recommended dose reductions. According to the Warnings and Precautions and Dose Modification sections of the Avastin® United States Prescribing Information, the following actions are recommended:
Perforation or Fistula: Discontinue BEV if perforation or fistula occurs.
Wound Healing: Discontinue BEV for wound dehiscence and wound healing complications requiring medical intervention
Hemorrhage: Discontinue BEV in patients with serious hemorrhage
Arterial Thromboembolic Events (ATE) (e.g., myocardial infarction, cerebral infarction): Discontinue BEV for severe ATE.
Venous Thromboembolic Events (VTE): Discontinue BEV for life-threatening (Grade 4) VTE, including pulmonary embolism
Hypertension: Monitor blood pressure and treat hypertension. Temporarily suspend BEV if not medically controlled. Discontinue BEV for hypertensive crisis or hypertensive encephalopathy.
Posterior Reversible Encephalopathy Syndrome (PRES): Discontinue BEV.
Proteinuria: Monitor proteinuria by dipstick urine analysis for the development or worsening of proteinuria with serial urinalyses during BEV therapy. Patients with a 2+ or greater urine dipstick reading should undergo further assessment with a 24-hour urine collection. Suspend BEV administration for ≥2 grams of proteinuria/24 hours and resume when proteinuria is <2 gm/24 hours. Discontinue BEV in patients with nephrotic syndrome.
Infusion Reactions: Stop BEV for severe infusion reactions and administer appropriate medical therapy.
3.8.6. Dose-Limiting Toxicity

### Part 1 Phase 1

Dose-limiting toxicity (DLT) is defined as the occurrence of any of the following AEs related to one of the drugs or the combination observed during Cycle 1, using NCI-CTCAE (v 4.03):
≥Grade 3 thrombocytopenia or Grade 2 thrombocytopenia with bleeding.
Grade 4 neutropenia or anemia lasting for more than 4 days.
Febrile neutropenia.
Any ≥Grade 2 neurological event lasting more than 4 days.
Grade 3 or 4 non-hematologic toxicity (excluding alopecia), lasting for more than 4 days despite adequate supportive therapy or preventing the next scheduled dose from being administered within 4 days of scheduled day; for ≥Grade 3 fatigue to be considered a DLT, it must be present for more than 7 days.

Subjects without DLT in Cycle 1 who do not receive 3 MRZ doses or 2 BEV doses within 5 weeks from first dose will not be evaluable for DLT and will be replaced.

### Part 2 Phase 2

If at any time after 3 subjects are enrolled, the incidence of AEs that fit the definition of DLT from Phase 1 occurs in >33% of the subjects, then enrollment will be paused. Available data will be reviewed and a decision regarding continuing to enroll subjects at the 0.8 mg/m² over 10 minutes dose in Phase 2 is agreed between the Sponsor and Investigators. Adjustment downward on dose or lengthening infusion duration will be considered and the Phase 2 portion restarted at the selected dose and infusion time.

### Part 3 Phase 2

The term DLT is not applicable to this portion of the study. DLAEs, defined as MRZ-related AEs 1) related to disturbances in the cerebellum (i.e., ataxia, dizziness, dysarthria, fall, gait disturbances) plus hallucinations of any grade or 2) Grade ≥2 other AEs will be used to determine if MRZ doses should be delayed, reduced, or discontinued.
3.8.7. Dose Escalation Process and MTD/MAD Determination

### Part 1 Phase 1 (Only)

Initially 3 subjects will be enrolled into a cohort, commencing with Cohort 1 and the doses shown in Table 36. Dose escalation will proceed as follows:
If none of the first 3 evaluable subjects in a dose cohort experience a DLT during Cycle 1, then enrollment into the next dose cohort can be initiated.
If ≥2 of the first 3 evaluable subjects in a dose cohort experience a DLT during Cycle 1, then the MTD has been exceeded and dose escalation will not proceed.
If 1 of the first 3 evaluable subjects in a dose cohort experiences a DLT during Cycle 1, then an additional 3 subjects will be enrolled into the same cohort.
If 1/6 evaluable subjects in the expanded 6-subject cohort experiences a DLT during Cycle 1, then the next higher dose cohort can be tested and enrollment of the next 3 subjects at the next higher dose level can be initiated.
If ≥2/6 evaluable subjects in the expanded 6-subject cohort experience a DLT during

Cycle 1, then the MTD has been exceeded and no further dose escalation will occur.

The MTD is defined as the dose level below the cohort where DLT is observed in at least 2 subjects in the same cohort during Cycle 1. Intermediate dosing levels may be explored if indicated. The dose of 0.8 mg/m² will not be exceeded and will be the MAD.

During the dose escalation phase of the protocol, if 2 DLTs are noted in the first 2 subjects of a cohort prior to the third subject being enrolled, the third subject will not be enrolled in that cohort. If there is 1 DLT in the first 3 subjects and the cohort is expanded and another DLT is noted prior to enrolling all 6 subjects in the cohort, further enrollment in that cohort will be halted. If during the dose expansion phase there are ≥3 DLTs in the first 6 or fewer subjects then the MTD will be reassessed by the Investigators and Sponsor.

Once the MTD/MAD has been identified, a cohort of at least 12 additional, evaluable subjects for a total of 36 subjects will be treated at the MTD/MAD to further confirm the safety and to assess preliminary activity for the combination treatment. This cohort may be used to determine the RP2D.
3.9. Method of Treatment Assignment

### Part 1 Phase 1

Treatments consist of IV doses of MRZ and BEV. Subjects will enter the study sequentially and be assigned to a cohort (dose level) based on the evaluation of subjects who were previously treated according to the dose escalation scheme. Once the RP2D is determined, subjects will be treated at the RP2D in the expansion cohort unless the Investigators and Sponsor agree to a lower dose for safety reasons.

### Part 2 Phase 2

Treatments consist of IV doses of MRZ. Subjects will enter the study sequentially.

### Part 3 Phase 2

Treatment consists of IV doses of MRZ and BEV. Subjects will enter the study sequentially.

### Concomitant Medications and Procedures

3.10. Permitted Concomitant Medications and Procedures

Concomitant medications to treat comorbid conditions and adverse events are permitted. Enzyme-inducing anti-epileptic drugs (EIAEDs) are allowed. Steroids are allowed and dosing is at the discretion of the Investigator. Consideration should be given to treating hallucinations with anti-psychotic drugs such as olanzapine or quetiapine and fatigue with stimulating agents such as methylphenidate.

In studies to date, MRZ has caused clinically significant nausea and vomiting requiring the use of antiemetics as therapy and also as prophylaxis. Therefore, both the therapeutic and prophylactic use of antiemetics is allowed in this study at the discretion of the Investigator.
3.11. Prohibited Concomitant Medications and Procedures

Medications to treat the underlying malignancy are not permitted and their use constitutes progressive disease and subjects must discontinue study treatment. Investigational agents of any kind are not permitted.
3.12. Required Concomitant Medications and Procedures

There are no required concomitant medications or procedures.

### Statistical Analyses

3.13. Overview

### Part 1 Phase 1

A 3+3 design will be utilized to determine the MTD/MAD for combination treatment of MRZ+BEV in each 28-day cycle. Subjects who do not have a DLT in the first cycle of a dose cohort will be replaced if they discontinue treatment with MRZ or BEV in Cycle 1 for any other reason. Subjects who miss a dose of MRZ or BEV or cannot receive all doses within 5 weeks from first dose during Cycle 1 and do not have a DLT will not be evaluable for DLT and will be replaced. After MTD/MAD has been determined in the dose-escalation part of the study, at least 12 additional subjects will be treated at the MTD/MAD in an expansion cohort to confirm the safety and assess the preliminary activity for the combination of MRZ+BEV administered up to a total of 36 subjects.

For all analyses by dose cohorts, the MTD/MAD confirmation cohort subjects will be combined with the corresponding dose cohort in the MTD/MAD determination phase as one single dose cohort.

### Part 2 Phase 2

Patients enrolled in the Phase 2 portion of the protocol will receive 0.8 mg/m² MRZ IV on Days 1, 8, and 15 of 28-day cycles. A minimum of 15 response-evaluable patients will be enrolled in Stage 1, and up to 15 additional response-evaluable patients will be enrolled in Stage 2, for a maximum of 30 response-evaluable patients. After the first 15 response-evaluable patients in the first stage have received 2 or more cycles of therapy, there will be a recommendation of whether to enroll the second stage based on an assessment of both safety and efficacy. If there are no safety concerns and clinical benefit is demonstrated with evidence of disease response, defined as at least one response (partial response (PR) or better) as determined by RANO criteria, in 15 response-evaluable patients, then 15 additional response-evaluable patients will be enrolled in Phase 2. Otherwise, there will be no further enrollment into the study.

Efforts will be made to ensure the correct number of patients is accrued, and enrollment will be carefully monitored and communicated with the sites. There may be instances where, as a result of simultaneous screening activities, patients may qualify for the study at the same time, resulting in slight over-enrollment.

### Part 3 Phase 2

A sample size of 40 eligible patients is based on wanting a reasonably precise estimate of median OS.

Efforts will be made to ensure the correct number of patients is accrued, and enrollment will be carefully monitored and communicated with the sites. There may be instances where, as a result of simultaneous screening activities, patients may qualify for the study at the same time, resulting in slight over-enrollment.
3.14. Study Population Definitions

All subjects who receive at least one dose of study medication (MRZ or BEV) will be considered enrolled in the study and will be in the Safety Population. All subjects who receive at least one dose of study medication and have at least 1 post dose PK sample will be in the PK Population. All subjects who receive at least 1 cycle of therapy and have at least 1 post treatment tumor assessment will be in the Activity (Efficacy) Population (Response-evaluable Population).
3.15. Sample Size

### Part 1 Phase 1

Up to 36 subjects will be enrolled in the MTD/MAD determination and confirmation (expansion cohort) parts of the study.

### Part 2 Phase 2

Fifteen response-evaluable patients will be enrolled in the first stage of a 2-stage design. If there is at least 1 PR or better and there are no safety concerns in the first 15 response-evaluable patients, then the second stage of 15 response-evaluable patients will be opened. If ≥5 patients respond by the end of the second accrual stage (n=30), the conclusion can be drawn that MRZ is promising, unless other considerations indicate otherwise.

The assumption for Phase 2 for sample size (for 30 patients) is a null hypothesis (Ho) that the true response rate is ≤5% versus the alternative hypothesis (Ha) that the true response rate is at least 20%. The significance level (i.e., the probability of rejecting Ho when it is true) is 0.05. The power (i.e., the probability of rejecting Ho when the alternative is true) is 80%.

### Part 3 Phase 2

A sample size of 40 patients is based on wanting a reasonably precise estimate of OS. Assuming 10 patients will be alive at the time of statistical analysis, there will be 30 deaths observed (i.e., 25% of the subjects are censored). The resulting 95% confidence interval (CI) is 7.2-14.8 months, with a width equal to 7.6 months, around an estimated median survival of 10 months.
3.16. Activity (Efficacy) Analysis

Tumor response including PD, progression-free survival (PFS), and overall survival (OS) will be assessed by the Investigators using RANO 2010 criteria. The overall confirmed response rate will be examined. The overall response rate, PFS, and OS will also be tabulated by dose cohorts. Results of Phase 1 and the two Phase 2 portions of the study will not be combined.
3.17. Pharmacokinetic Analysis (Part 1 Phase 1 Only)

Non-compartmental analyses will be performed. The following PK parameters will be calculated: maximum observed blood drug concentration (Cₘₐₓ), time of maximum blood concentration (Tₘₐₓ), elimination half-life (T_{1/2}), area under the blood concentration-time curve (AUC_{0-inf}), clearance (CL), and volume of distribution (Vd).

Blood concentrations and computed PK parameters for MRZ will be listed and summarized by cohort (mean, geometric mean, standard deviation, coefficient of variation, minimum, maximum and number of observations).
3.18. Pharmacodynamic Analysis (Part 1 Phase 1 Only)

PD analysis will include change in proteasome activities (whole blood lysates and PBMC lysates) by comparing pre-drug and post drug levels on Days 1, 8, and 15 (i.e., for each dose) of Cycle 1 and Days 1 and 15 (i.e., for the first and last doses) of each cycle thereafter.

### Adverse Events

3.19. Monitoring, Recording and Reporting of Adverse Events

An adverse event (AE) is any noxious, unintended, or untoward medical occurrence that may appear or worsen in a subject during the course of a study. It may be a new intercurrent illness, a worsening concomitant illness, an injury, or any concomitant impairment of the subject's health, including laboratory test values (as specified by the criteria in Section 10.3), regardless of etiology. Any worsening (i.e., any clinically significant adverse change in the frequency or intensity of a pre-existing condition) should be considered an AE.

Overdose (accidental or intentional), abuse, withdrawal, sensitivity or toxicity to study treatment should be reported as an AE. If an overdose is associated with an AE, the overdose and AE should be reported as separate terms. Any sequelae of an accidental or intentional overdose of an investigational product should be reported as an AE on the AE CRF. If the sequelae of an overdose are an SAE, then the sequelae must be reported on an SAE report form and on the AE CRF. The overdose resulting in the SAE should be identified as the cause of the event on the SAE report form and CRF but should not be reported as an SAE itself. Medication errors, defined as an overdose with >105% of drug administered, or underdose, defined as <95% of dose administered, are to be reported as AEs. Prescribed dose reductions for AEs are not considered medication errors.

All subjects will be monitored for AEs during the study. Assessments may include monitoring of any or all of the following parameters: the subject's clinical symptoms, laboratory, pathological, radiological or surgical findings, physical examination findings, or findings from other tests and/or procedures.

All SAEs will be recorded by the Investigator from the time the subject signs informed consent until 28 days after the last dose of study treatment and those SAEs made known to the Investigator at any time thereafter that are suspected of being related to study treatment. AEs are recorded from the start of the first infusion of study treatment. AEs occurring before the first infusion of IP are considered medical history and should be recorded on the medical history CRF. AEs and serious adverse events (SAEs) will be recorded on the AE page of the CRF and in the subject's source documents. Evaluation of Adverse Events

A qualified Investigator will evaluate all adverse events as to:
3.19.1. Seriousness

A serious adverse event (SAE) is any AE occurring at any dose that:
Results in death;
Is life-threatening (i.e., in the opinion of the Investigator, the subject is at immediate risk of death from the AE);
Requires inpatient hospitalization or prolongation of existing hospitalization (hospitalization is defined as an inpatient admission, regardless of length of stay);
Results in persistent or significant disability/incapacity (a substantial disruption of the subject's ability to conduct normal life functions);
Is a congenital anomaly/birth defect;
Constitutes an important medical event.

Important medical events are defined as those occurrences that may not be immediately life threatening or result in death, hospitalization, or disability, but may jeopardize the subject or require medical or surgical intervention to prevent one of the other outcomes listed above. Medical and scientific judgment should be exercised in deciding whether such an AE should be considered serious.

Events not considered to be SAEs are hospitalizations for:
A standard procedure for protocol therapy administration. However, hospitalization or prolonged hospitalization for a complication of therapy administration will be reported as an SAE.
Routine treatment or monitoring of the studied indication not associated with any deterioration in condition.
The administration of blood or platelet transfusion as routine treatment of studied indication. However, hospitalization or prolonged hospitalization for a complication of such transfusion remains a reportable SAE.
A procedure for protocol/disease-related investigations (e.g., surgery, scans, endoscopy, sampling for laboratory tests, bone marrow sampling). However, hospitalization or prolonged hospitalization for a complication of such procedures remains a reportable SAE.
Hospitalization or prolongation of hospitalization for technical, practical, or social reasons, in absence of an AE.
A procedure that is planned (i.e., planned prior to starting of treatment on study); must be documented in the source document and the CRF. Hospitalization or prolonged hospitalization for a complication remains a reportable SAE.
An elective treatment of or an elective procedure for a pre-existing condition unrelated to the studied indication.
Emergency outpatient treatment or observation that does not result in admission, unless fulfilling other seriousness criteria above.

Severity/Intensity

For both AEs and SAEs, the Investigator must assess the severity/intensity of the event.

The severity/intensity of AEs will be graded based upon the subject's symptoms according to the current active minor version of the Common Terminology Criteria for Adverse Events ( ).

The term “severe” is often used to describe the intensity of a specific event (as in mild, moderate or severe myocardial infarction); the event itself, however, may be of relatively minor medical significance (such as severe headache). This criterion is not the same as “serious” which is based on subject/event outcome or action criteria associated with events that pose a threat to a subject's life or functioning.

Seriousness, not severity, serves as a guide for defining regulatory obligations.
3.19.2. Causality

The Investigator must determine the relationship between the administration of study treatment and the occurrence of an AE/SAE as Not Suspected or Suspected as defined below:
Not suspected: Means a causal relationship of the adverse event to study treatment administration is unlikely or remote, or other medications, therapeutic interventions, or underlying conditions provide a sufficient explanation for the observed event.
Suspected: Means there is a reasonable possibility that the administration of study treatment caused the adverse event. ‘Reasonable possibility’ means there is evidence to suggest a causal relationship between the study treatment and the adverse event.

Causality should be assessed and provided for every AE/SAE based on currently available information. Causality is to be reassessed and provided as additional information becomes available. For regulatory purposes, it is the Sponsor that is responsible for making the final causality assessment.
3.19.3. Duration

For both AEs and SAEs, the Investigator will provide a record of the start and stop dates of the event. For AEs that become SAEs, the start date of the SAE will be when the seriousness criteria are met. The original AE will have a stop date the same as the start date of the SAE. The SAE will have a stop date of when the seriousness criteria are no longer met. If the AE continues after the seriousness criteria are no longer met, then a new AE will be recorded with a start date the same as the SAE stop date and a stop date when the AE is completely resolved. In all cases, the AE must have the same verbatim term throughout. Within the duration of the SAE or AE, the maximum grade should be used to categorize severity.
3.19.4. Action Taken

The Investigator will report the action taken with each study drug as a result of an AE or SAE, as applicable (e.g., discontinuation, interruption, or reduction of study treatment, as appropriate) and report if concomitant and/or additional treatments were given for the event.
3.19.5. Outcome

The Investigator will report the outcome of the event for both AEs and SAEs. All SAEs that have not resolved upon discontinuation of the subject's participation in the study must be followed until recovered, recovered with sequelae, returned to baseline, stabilized, or died (due to the SAE or due to another cause).
3.20. Abnormal Laboratory Values

An abnormal laboratory value is to be considered an AE if the abnormality:
results in discontinuation from the study;
requires treatment, modification/interruption of IP dose, or any other therapeutic intervention; or
is judged to be of significant clinical importance.

Regardless of severity grade, only laboratory abnormalities that fulfill a seriousness criterion need to be documented as a serious adverse event.

If a laboratory abnormality is one component of a diagnosis or syndrome, then only the diagnosis or syndrome should be recorded on the AE page of the CRF. If the abnormality was not a part of a diagnosis or syndrome, then the laboratory abnormality should be recorded as the AE. If possible, the laboratory abnormality should be recorded as a medical term and not simply as an abnormal laboratory result (e.g., record thrombocytopenia rather than decreased platelets).

### Discontinuations

The following events are considered sufficient reasons for discontinuing a subject from the investigational product and/or from the study:
Protocol Violation
Non-Compliance
Adverse Event
Subject Developed a DLT
Subject Decision
Withdrew Consent
Investigator Decision
Disease Progression
Pregnancy
Death
Other

### Example 3

### Full Enrollment Results from the Phase 1/2, Multicenter, Open-Label Study of Marizomib (MRZ)±Bevacizumab (BEV) in Recurrent WHO Grade IV Malignant Glioma (Glioblastoma, rGBM)

Example 3 represents an abstract relating to Examples 1 and 2.

MRZ—an irreversible, brain-penetrant, pan-proteasome inhibitor with anti-glioma preclinical activity—was evaluated in BEV-naive rGBM patients. METHODS: Phase 1 (P1) MRZ+BEV, 3+3 MRZ dose-escalation (N=6, 3, 3 at 0.55, 0.7, 0.8 mg/m²) followed by dose-expansion (N=24, 0.8 mg/m²). Phase 2 (P2) MRZ monotherapy (N=30, 0.8 mg/m²). Treatments (IV, 28-day (D) cycles): MRZ (10min infusion) D1, 8, 15; BEV (10 mg/kg) D1, 15. Tumor response (RANO criteria) every other cycle; MRZ and BEV PK, and proteasome inhibition in blood evaluated in P1. RESULTS: as of 14Apr. 2017: P1 mean age 55 yrs, 64% male, mean treatment duration 5.3 cycles, 1 patient active; P2 56 yrs, 57% male, 2.5 cycles, 6 patients active. One DLT (fatigue) in P1 at 0.55 mg/m², no other DLTs. P1 treatment-related AEs (Grade ≥3 in ≥2 patients): hypertension, headache, confusional state, fatigue, hallucination, proteinuria; three Grade 4 SAEs (appendicitis perforated, depressed level of consciousness, not-related; blindness, BEV-related), three Grade 5 SAEs (2 PD, not-related; intracranial hemorrhage, BEV-related). P2 treatment-related AEs (Grade ≥3 in ≥2 patients): fatigue, hallucination, lethargy; one Grade 4 SAE (hallucination). In P1, overall response (>PR by RANO) 44% (16/36) including 1 CR, 15 PR; overall survival (OS) at 6/9/12 months (mos) 75/60/39%, median 9.4mos; OS 68/45/15% (median 7.2 mos) in unmethylated MGMT (uMGMT, N=22), 78/78/67% (median not reached) in methylated MGMT (N=10). In P2: 1 PR, 6 SD; 4 patients (3 SD, 1 PR) ongoing at 5-10 cycles. P1 patients experiencing ≥1 CNS-related AEs (any grade: ataxia/balance disorder/dizziness/dysarthria/fall/gait disturbance/hallucination) have increased OS (83/74/45%, median 11.4 mos, N=23) versus patients without these AEs (59/34/25%, median 6.3mos, N=13). CONCLUSIONS: MRZ monotherapy and MRZ+BEV active in rGBM overall and in uMGMT. Possible therapeutic improvement in patients experiencing CNS AEs will be explored in ongoing P2 MRZ+BEV extension allowing intra-patient MRZ dose-escalation if no CNS AE in first cycle (0.8 mg/m²).

### Example 4

### Full Enrollment Results from the Phase 1/2, Multicenter, Open-Label Study of Marizomib (MRZ)±Bevacizumab (BEV) in Recurrent WHO Grade IV Malignant Glioma (Glioblastoma, rGBM)

This example gives a part I Phase 1 dose escalation combination study followed by a part II marizomib monotherapy study and a part III intra-patient dose escalation combination study.

### Study Objective and Design

The primary objective was to determine the maximum tolerated dose and recommended phase II dose of marizomib+bevacizumab. The secondary objective was to evaluate the safety and activity of marizomib+bevacizumab.

An exploratory objective was to evaluate the baseline proteasome activity, marizomib and bevacizumab PK, marizomib neurological coordination (SARA), and quality of life assessment (FACT-Cog/FACT-Br).

### Methods

The clinical trial was a Phase 1, dose escalation (3+3 design) followed by dose-expansion at recommended Phase 2 dose (RP2D). In part I, MRZ+BEV, the MRZ dose escalation was to 0.8 mg/m². Three dose escalation cohorts were used; marizomib 0.55 (6 pts), 0.7 (3 pts), and 0.8 mg/m² (3 pts); dose-expansion 0.8 mg/m² (24 pts). The maximum dose level of MRZ was set at 0.8 mg/m² due to CNS adverse events observed in earlier studies. In part II, MRZ monotherapy was evaluated with MRZ at 0.8 mg/m².

Marizomib was infused intravenous (IV; 10 min) on Days 1, 8, & 15; bevacizumab was infused IV at 10 mg/kg on days 1 & 15. The drugs were infused on 28-Day cycles. Tumor response is assessed every other cycle by RANO criteria. Blood marizomib pharmacokinetic parameters were assessed on Day 8, serum bevacizumab pharmacokinetic parameters were assessed on days 1 and 15; blood proteasome inhibition was assessed on days 1 and 15 every cycle. Table 37 gives the treatment parameters of the present study.

TABLE 37
Treatment Parameters of Grade IV MG Study

| | | | IV MRZ | BEV IV |
|---|---|---|---|---|
| | | | (mg/m²) 10 | (mg/kg) |
| | | # Patients | min infusion Days 1, | Days 1, 15 |
| | Cohorts | per cohort | 8, 15, q28 days | q 28 days |
| Part 1 | 1 | 6 | 0.55 | 10 |
| | 2 | 3 | 0.7 | 10 |
| | 3 | 3 | 0.8 | 10 |
| | 4 | 24 | Expansion of RP2D | 10 |
| Part 2 | 5 | 30 | 0.8 | None |
| | Monotherapy | | | |
| Part 3 | 6 | 40 | Intrapatient Dose | 10 |
| | | | Escalation | |
| | | | 0.8→1.0→1.2 | |

The key eligibility criteria included patients over 18 years of age, with histological evidence of grade IV malignant glioma in first or second relapse with clear progressive disease. Participants much have completed standard radiation therapy and temozolomide. Additional criteria included no prior proteasome inhibitor (including marizomib) or anti-angiogenic therapies, and a Karnofsky Performance Score greater than or equal to 70. Criteria also included that the patient be at least four weeks from surgical resection and 12 weeks from the end of radiotherapy. Table 38 gives the demographics of the study participants.

TABLE 38
Demographics and Baseline Disease Characteristics
of Study Participants

| | | | Part 2 | | | |
|---|---|---|---|---|---|---|
| | Part 1 | | MRZ | | Part 3 | |
| | MRZ + BEV | | Monotherapy | | MRZ + BEV | |
| | (N = 36) | | (N = 30) | | (N = 40) | |
| Age | | | | | | |
| Median (range) | 55 | (27, 76) | 58 | (25, 80) | 56 | (20, 75) |
| Gender | | | | | | |
| Female | 13 | (36%) | 13 | (43%) | 20 | (50%) |
| Male | 23 | (64%) | 17 | (57%) | 20 | (50%) |
| KPS (%) | | | | | | |
| 100 | 6 | (17%) | 2 | (7%) | 3 | (8%) |
| 90 | 11 | (31%) | 15 | (50%) | 14 | (35%) |
| 80 | 14 | (39%) | 9 | (30%) | 16 | (40%) |
| 70 | 5 | (14%) | 4 | (13%) | 6 | (15%) |
| Time since Initial | | | | | | |
| Diagnosis | | | | | | |
| (months) | | | | | | |
| Median (range) | 10.5 | (5, 41) | 10.7 | (0.7, 36) | 11.2 | (0.1, 88) |
| MGMT Meth- | | | | | | |
| ylation | | | | | | |
| Status (%) | | | | | | |
| Methylated | 10 | (28%) | 7 | (23%) | In progress | |
| Unmethylated | 22 | (61%) | 19 | (63%) | | |
| Number of Prior | | | | | | |
| Regimens (%) | | | | | | |
| 1 | 23 | (64%) | 15 | (50%) | 25 | (62%) |
| 2 | 10 | (28%) | 13 | (43%) | 10 | (25%) |
| 3 | 3 | (8%) | 1 | (3%) | 5 | (12%) |

### Results

Thirty-six patients enrolled with a median age 55 years (27-76), 64% were male, Karnofsky Score >70. Duration of dosing was 0.25-15 months to date; treatment is ongoing in 3 pts. Marizomib and bevacizumab was well tolerated.

Table 39 summarizes treatment-emergent adverse events (1TEAEs). A majority of the most frequent TEAEs are grades 1 & 2. Study treatment-related Grade ≥3 adverse events include fatigue, headache, hypertension, hallucination, confusional state, ataxia, optic nerve disorder, insomnia, delusion, and hyponatremia. One grade 4 hallucination was noted. Further, there are similar MRZ-related TEAEs (Part II, MRZ monotherapy) between the studies, BEV-related TEAES that noted a higher incidence (Part I, MRZ+BEV) include hypertension, dysphonia, and epistaxis. The CNS-related toxicities unique to MRZ include hallucination, confusion, ataxia, etc.

TABLE 39
Most Freauent Treatment-Related Adverse Events

| | Part 1 (N = 36) | | | | | Part 2 (N = 30) | | | | |
|---|---|---|---|---|---|---|---|---|---|---|
| | MRZ + BEV | | | | | MRZ Monotherapy | | | | |
| Adverse Event | Grade | | | | | | | | | |
| In >20%, by Worst Grade | Total | 1 | 2 | 3 | 4 | Total | 1 | 2 | 3 | 4 |
| Fatigue | 26 | 12 | 11 | 3 | — | 20 | 8 | 11 | 2 | — |
| Nausea | 23 | 12 | 11 | — | — | 11 | 7 | 2 | 2 | — |
| Headache | 19 | 7 | 7 | 5 | — | 14 | 10 | 3 | 1 | — |
| Vomiting | 19 | 11 | 8 | — | — | 10 | 8 | — | 2 | — |
| Hypertension | 17 | | 4 | 12 | 1 | 4 | 1 | 1 | 2 | — |
| Insomnia | 4 | 1 | 2 | 1 | — | 1. | 7 | 5 | 1 | — |
| Hallucination | 13 | 6 | 5 | 2 | — | 12 | 6 | 4 | 1 | 1 |
| Dysphonia | 11 | 10 | 1 | — | — | 1 | 1 | — | — | — |
| Diarrhea | 10 | 10 | — | — | — | 7 | 6 | — | 1 | — |
| Epistaxis | 10 | 9 | 1 | — | — | 2 | 2 | — | — | — |
| Infusion Site Reaction | 10 | 8 | 2 | — | — | 5 | 3 | 2 | — | — |
| (includes edema, pain, | | | | | | | | | | |
| phlebitis, redness) | | | | | | | | | | |
| Dizziness | 10 | 7 | 3 | — | — | 5 | 4 | 1 | — | — |
| Confusional State | 10 | 2 | 5 | 3 | — | 7 | 3 | 4 | — | — |
| Fall | 10 | 4 | 5 | 1 | — | 4 | 3 | 1 | — | — |
| Constipation | 9 | 8 | 1 | — | — | 10 | 10 | — | — | — |
| Anemia | 8 | 7 | 1 | — | — | 6 | 5 | 1 | — | — |
| Hypokalemia | 8 | 6 | 1 | 1 | — | 9 | 7 | 2 | — | — |
| Ataxia | 8 | 4 | 3 | 1 | — | 8 | 3 | 5 | — | — |
| Hyperglycemia | 8 | 1 | 5 | 2 | — | 7 | 2 | 4 | 1− | |
| Platelet Count Decreased | 8 | 4 | 4 | — | — | 7 | 7 | — | — | — |

### Part I—MRZ+BEV Dose-Escalation

FIG. 20 shows a waterfall plot that depicts tumor response by RANO in 33 radiologically-evaluable patients. The overall response rate (ORR) was 44% including 1 complet response and 15 confirmed partial responses which is higher than previously published values for BEV monotherapy. Table 40 displays the Part I MRZ+BEV response rate by response assessment in neuro-oncology (RANO) criteria.

TABLE 40
Part 1 MRZ + BEV Response Rate by Response
Assessment in Neuro-Oncology (RANO) Criteria

| RANO Response | N = 36 |
|---|---|
| CR | 1 |
| PR (confirmed) | 15 |
| SD | 11 |
| PD | 6 |
| NE | 3 |

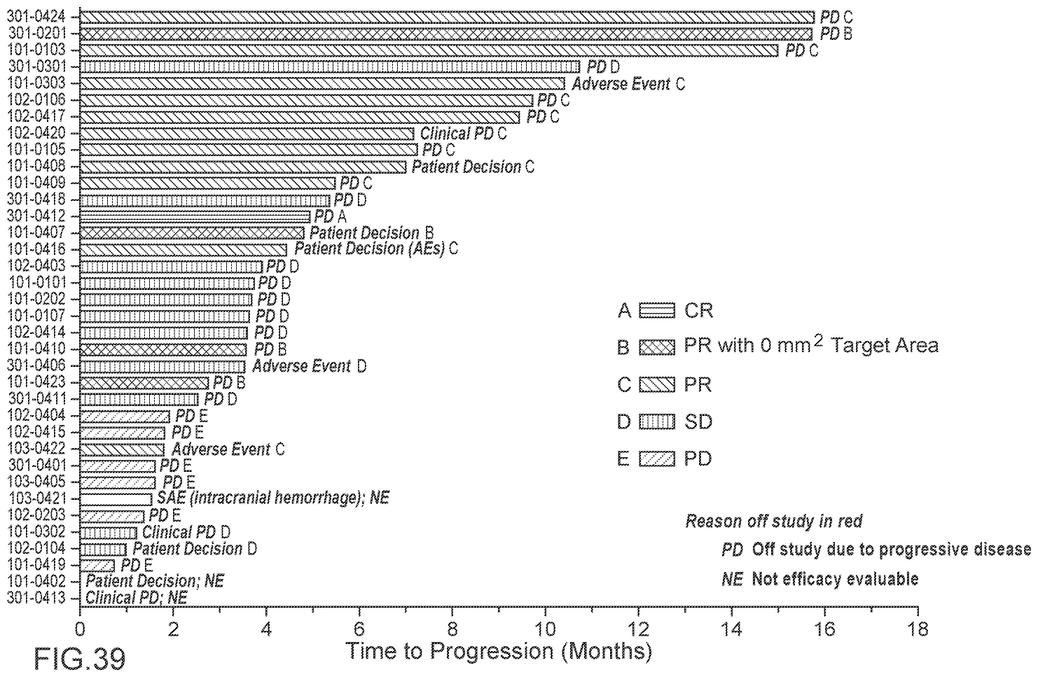

FIG. 39 is a breakdown of patient time duration on treatment in the part I MRZ+BEV dose escalation. All patients completed part I. Dose-escalation required 6 patients in cohort 1 (0.55 mg/m²) due to dose-limiting toxicities (DLTs) in the form of fatigue. No other DLTs were noted in dose-escalation and dose-expansion was conducted at 0.8 mg/m² BEV. In summary 27 of 36 patients are off study due to progressive disease/clinical progression with 3 due to AE, 1 due to SAE (intracranial hemorrhage, death, BEV-related) and 5 due to patient decision.

FIG. 30A shows a plot of the progression free survival (PFS) percent as a function of time for all patients. FIG. 30B shows a plot of the overall survival (OS) percent as a function of time for all patients. Table 41 shows the PFS and OS in MRZ+BEV studies and demonstrates the activity of the combination. Without wishing to be bound by theory, unmethylated MGMT promoter is a biomarker of poor prognosis in malignant glioma. Patients with unmethylated MGMT promoter can be more likely to suffer recurrent disease, and for recurrence to occur more quickly than in patients with methylated MGMT promoter. For instance, patients with unmethylated MGMT promoter who are treated with the standard of care (temozolomide and radiotherapy) can be more likely to relapse. Interestingly, PFS and OS in unmethylated MGMT patients is higher than those published with BEV monotherapy. Patients with unmethylated MGMT promoter had similar progression free survival (PFS) as patients with methylated MGMT. In addition, methylated MGMT is associated with better overall survival (OS) in comparison with unmethylated

TABLE 41
Part 1 MRZ + BEV Progression-Free Survival (PFS) and
Overall Survival (OS) by MGMT Promoter Methylation Status

| | PFS | | | | OS | | | |
|---|---|---|---|---|---|---|---|---|
| | # | Median | 6 mo. | 9 mo. | # | Median | 9 mo. | 12 mo. |
| Patients | Censored | (months) | (%) | (%) | Censored | (months) | (%) | (%) |
| All | 5 | 3.9 | 34 | 24 | 10 | 9.4 | 60 | 37 |
| (N = 36) | | | | | | | | |
| uMGMT | 1 | 3.7 | 36 | 26 | 2 | 7.2 | 36 | 18 |
| Unmethylated | | | | | | | | |
| (N = 22) | | | | | | | | |
| mMGMT | 3 | 4.8 | 29 | 15 | 4 | 12.6 | 78 | 56 |
| Methylated | | | | | | | | |
| (N= 10) | | | | | | | | |

### Part II—MRZ Monotherapy

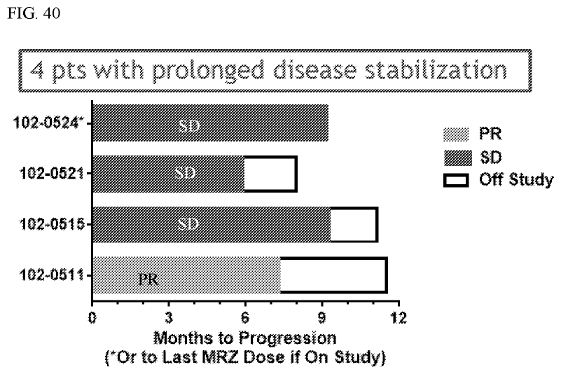

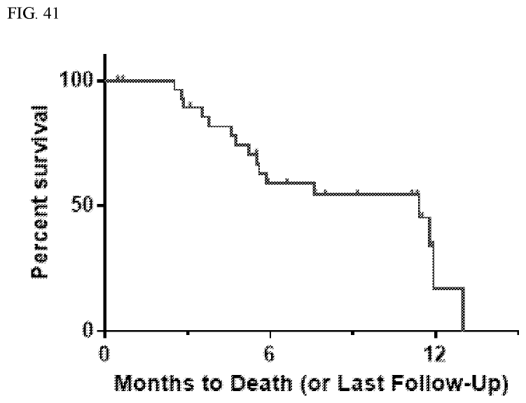

Most patients on marizomib monotherapy demonstrated rapid progression with the exception of 4 patients who demonstrated prolonged disease stabilization as shown in FIG. 40. All patients had documented progressive disease at the start of the study. Of the 30 patients enrolled, they demonstrated 1 partial response (PR0 and 8 stable disease (SD). 2 additional patients had no or little viable tumor upon re-surgery; appearing to be pseudoprogression. FIG. 41 shows a plot of overall survival (OS) for patients treated with marizomib monotherapy. The median overall survival (OS) was 11.4 months based on 16 events with an interesting OS signal (median follow-up 9.2 months).

TABLE 42
Part II MRZ Monotherapy Overall Survival (OS)
OS (N = 30, 14 censored)

| 6 mo % | 9 mo % | 12 mo % |
|---|---|---|
| 59 | 54 | 17 |

TABLE 43
CNS Adverse Events in MRZ + BEV

| Adverse Event (N) | Number of Patients (Worst Grade/Patient) | | |
|---|---|---|---|
| MRZ Dose (mg/m²) | MRZ + BEV (N = 36) | | |
| Grade | 1 | 2 | 3 |
| Ataxia | 5 | 2 | 1 |
| Dizziness/Balance Disorder | 7 | 3 | — |
| Dysarthria | 4 | 2 | 1 |
| Fall | 4 | 5 | 1 |
| Gait disturbance | 5 | — | — |
| Hallucination | 6 | 5 | 2 |

### Part III—Intra-Patient Dose Escalation

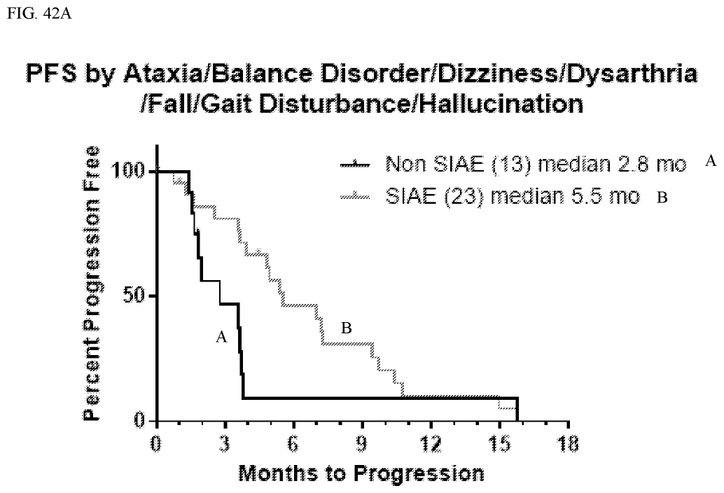

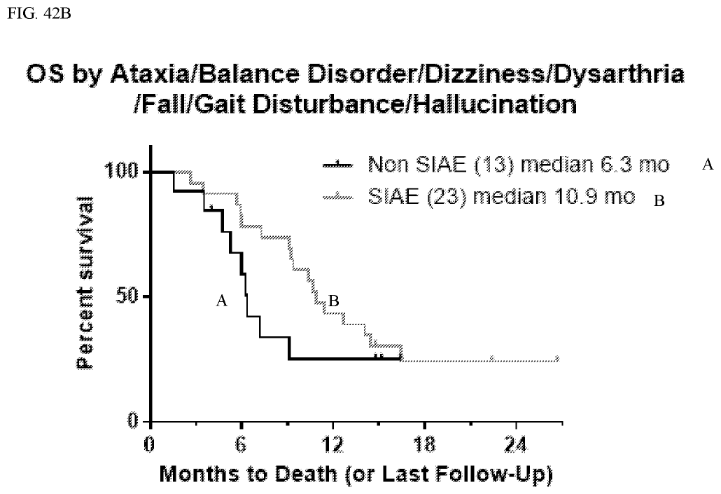

An adverse event of special interest (SIAE) was noted as MRZ+BEV patients with hallucinations tended to have better tumor response. Nearly two-thirds of patiens who experienced certain CNS AEs tended to have improved PFS and/or OS (Table 43). A systemic data analysis revealed a constellation of CNS adverse effects associated with improved PFS (FIG. 42A) and OS (FIG. 42B). Not wishing to be bound by theory, these special interest adverse events (i.e. ataxia, dizziness/balance disorder, dysarthria, fall, gait disturbance, hallucination) may be indicative of greater amounts of MRZ getting to the brain and/or tumor and/or cerebellum.

All subjects were to receive IV MRZ infusion and IV BEV infusion. MRZ was administered as a 10 minute IV infusion on Days 1, 8 and 15 of every 28-day cycle using an intra-patient dose escalation. The starting dose was 0.8 mg/m². In a 1st cycle MRZ was administered at 0.8 mg/m² plus BEV, if no dose limiting adverse events (DLAEs) were noted MRZ was escalated to at 1.0 mg/m² plus BEV. In a 2nd cycle MRZ was administered at 1.0 mg/m² plus BEV, if no DLAEs were noted MRZ was escalated to at 1.2 mg/m² plus BEV. For the purposes of this study a DLAE was defined as MRZ-related adverse events (AEs) which are related to disturbances in the cerebellum (i.e. ataxia, dizziness, balance disorder, dysarthria, fall, gait disturbances) plus hallucinations of any grate, or Grade ≥2 of other adverse events.

### Results

Of the first 35 patients eligible for dose escalation (completed cycle 1 at 0.8 mg/m²), a total of 10 were escalated to the 1.0 mg/m² dose, meaning that the remaining patients experienced either a special interest adverse event and/or a grade ≥2 that prohibited dose-escalation. Only 2 of these 10 patients completed the full second treatment cycle at 1.0 mg/m². One subject had treatment held after completing the second cycle and subsequently came off the study due to PD, and one subject was dose-reduced to 0.8 mg/m² at third cycle, day 1. The other 8 patients required does reduction in the second cycle or had treatment held for adverse effects and came off the study due to PD (1 came off the study for adverse events). Reasons for dose reductions included vomiting, nausea, headache, fatigue, ataxia/gait disturbance, confusion, hallucinations, agitation and adverse events ranging from grades 1-3 in severity. No patients reached or were escalated to 1.2 mg/m². This data suggested that the MTD for MRZ in combination with BEV in recurrent GMB patient is 0.8 mg/m².

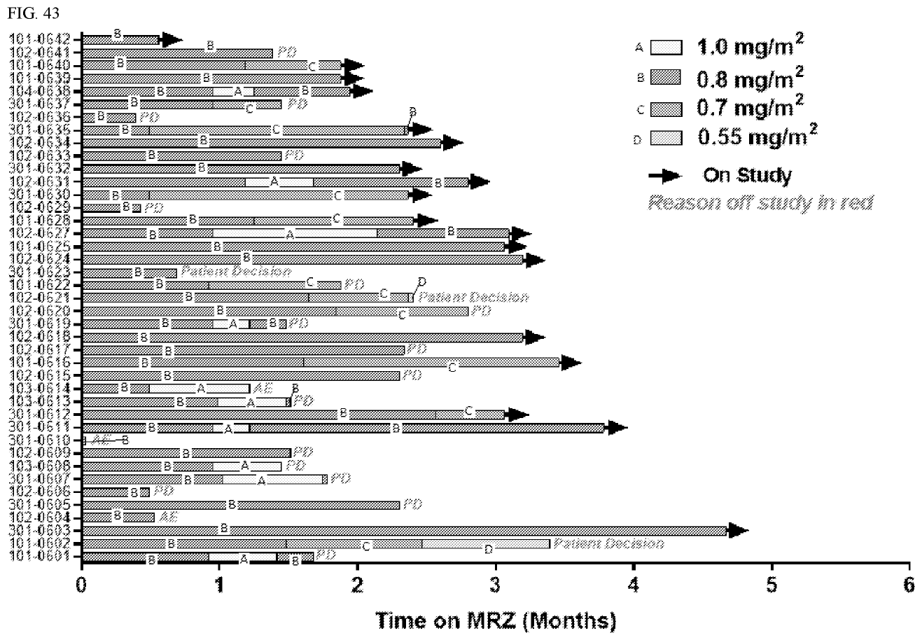

FIG. 43 is a breakdown of patient time on treatment in the part III intra-patient dose escalation. In summary, the preliminary data for time on MRZ+BEV treatment showed a fully enrolled N=41, with 19 patients remaining on the study and 22 patients off study (17 PD, 2 AE, and 3 patient decision). It is noted that 10 patients dose-escalated to 1.0 mg/m² (denoted [A]). Thus, although CNS adverse events are generally short lasting, reversible, and alleviated with dose reductions and medical management it is suggested that the 1.0 mg/m² dose is not tolerable in patients treated at progression, supporting 0.8 mg/m² as the recommended dosage.

### Example 5

### Phase 1, Multicenter, Open-Label Study of Marizomib (MRZ) with Temozolomide (TMZ) and Radiotherapy (RT) in Newly Diagnosed WHO Grade IV Malignant Glioma (Glioblastoma, ndGBM)

This example gives a study for newly diagnosed WHO Grade IV malignant glioma patients to determine whether marizomib (MRZ) will improve the treatment of newly diagnosed glioblastoma patients by delaying the growth of the cancer, reducing the size of the tumor, and/or improving survival. Marizomib (MRZ) was added to standard-of-care treatments of radiotherapy (RT) and temozolomide (TMZ).

### Study Objective and Design

The primary objective was to determine the MRZ maximum tolerated dose (MTh) and recommended phase 2 dose (RP2D) for both concomitant treatment (MRZ+TMZ+RT) and adjuvant treatment (MRZ+TMZ).

Other exploratory objectives include confirming the MRZ RP2D for concomitant and adjuvant treatment in an expanded group of patients, assess adverse events during concomitant and adjuvant treatment, evaluate the activity (overall survival [OS] and progression-free survival [PFS]) of MRZ+TMZ+RT, MRZ pharmacokinetics, and assess marizomib neurological coordination using the Scale for the Assessment and Rating for Ataxia (SARA).

### Methods

The clinical trial was a dose-escalation study (Stage 1, MRZ+TMZ+RT or MRZ+TMZ) consisting of two arms—concomitant and adjuvant—in each, MRZ added to standard of care at increasing IV doses in a 3+3 design, and a dose expansion study at RP2D (Stage 2, RP2D MRZ+TMZ+RT followed by RP2D MRZ+TMZ) first with concomitant and then with adjuvant standard of care.

In the experimental Stage 1 Concomitant Treatment (MRZ+TMZ+RT) Drug MRZ dose ranges from 0.55 mg/m² to 1.0 mg/m² given IV over 10 minutes on days 1, 8, 15, 29, and 36 during concomitant treatment. MRZ dose ranges from 0.55 to 1.0 mg/m² given IV over 10 minutes on Days 1, 8, 15 every 28 days during adjuvant treatment. TMZ will be administered once daily, 7 days/week, for 6 weeks, starting on Day 1, at a dose of 75 mg/m² during concomitant treatment. TMZ will be administered once daily on days 1-5 every cycle (28 day cycles, up to 12 cycles), dose range 150 to 200 mg/m² during adjuvant treatment. Radiation (RT) will be administered once daily 5 days/week, for 30 doses over 6 weeks to a total dose of 60 Gy, staring on Day 1 during concomitant treatment. Patients who complete concomitant treatment may continue on to adjuvant treatment, in the adjuvant cohort RT and TMZ treatment is received outside of protocol therapy.

In the experimental Stage 2 dose expansion 18 additional evaluable patients were enrolled in a cohort in which concomitant treatment (MRZ+TMZ+RT) is followed by adjuvant treatment (MRZ+TMZ) to confirm the MTD for each treatment regime as determined in the dose-escalation (Stage 1), and to assess preliminary activity of the recommended Phase 2 dose (RP2D, 0.8 mg/m²). Table 44 and Table 45 give the treatment parameters of the present study and demographics of study participants.

TABLE 44
Enrollment Summary of Patients by Stage of Study

| | | | | |
|---|---|---|---|---|
| | Cohort 1 | Cohort 2 | Cohort 3 | Cohort 4 |
| | 0.55 | 0.7 | 0.8 | 1.0 |
| Total | mg/m² | mg/m² | mg/m² | mg/m² |

Stage 1 Dose Escalation - Concomitant

| | | | | | |
|---|---|---|---|---|---|
| # of Subjects | 15 | 3 | 3 | 3 | 6 |
| Enrolled | | | | | |
| # of Subjects | 4 | 0 | 1 | 1 | 2 |
| Active | | | | | |

Stage 1 Dose Escalation - Adjuvant

| | | | | | |
|---|---|---|---|---|---|
| # of Subjects | 18 | 3 | 6 | 3 | 6 |
| Enrolled | | | | | |
| # of Subjects | 9 | 1 | 2 | 2 | 4 |
| Active | | | | | |

Stage 2 - Expansion (enrollment ongoing)

| | | Cohort 6 |
|---|---|---|
| | Total | 0.8 mg/m² |
| # of Subjects Enrolled | 18 | 11 |
| # of Subjects Active | 10 | 10 |

TABLE 45
Demographics and Baseline Disease Characteristics of Study Participants
N = 33 (dose-escalation)

| Age | | |
|---|---|---|
| Median (range) | 58.0 | (31, 75) |
| Sex (%) | | |
| Female | 9 | (27%) |
| Male | 24 | (73%) |
| KPS (%) | | |
| 100 | 5 | (15%) |
| 90 | 21 | (64%) |
| 80 | 5 | (15%) |
| 70 | 2 | (6%) |
| MGMT Methylation Status (%) | | |
| Methylated | 8 | (24%) |
| Unmethylated | 9 | (27%) |
| Extent of Surgical Resection (%) | | |
| Complete | 9 | (27%) |
| Partial | 20 | (61%) |
| Biopsy | 2 | (6%) |

### Results

Table 46 is a summary of treatment-related adverse events, all grades, in dose escalation (concomitant and adjuvant patients combined) in the three lower dose cohorts (0.55, 0.7, 0.8 mg/m²; N=21). Table 47 is a summary of treatment-related adverse events, all grades, in dose escalation (concomitant and adjuvant patients combined) comparing three lower dose cohorts with a highest dose cohort (1.0 mg/m²; N=12). A higher proportion of treatment-related adverse events is noted at 1.0 mg/m² in comparison with lower dose cohorts for both commonly observed (fatigue, etc.) as well as for CNS toxicities (hallucination, etc.). Not wishing to be bound by theory, CNS treatment-related adverse events may be due to greater brain levels of MRZ.

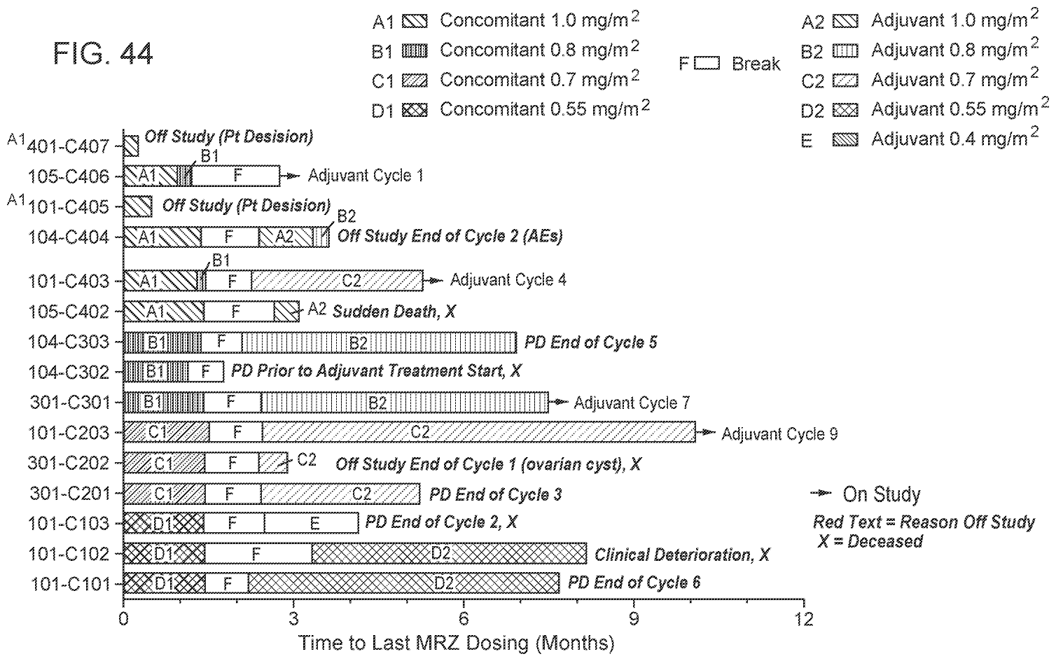

FIG. 44 is a swimmer plot of concomitant cohort (Stage 1) patients in dose-escalation with dose levels labeled, concomitant treatment (solid bars) followed by break (open bars) followed by adjuvant treatment (hatched bars). No dose-limiting toxicities (DLTs) were encountered until cohort 4 (1 mg/m²) in dose-escalation. DLTs included CNS toxicities (ataxia, hallucination, dysphasia, confusion) and most patients were able to continue on protocol with dose delay and/or reduction. As an enrollment summary; 15 enrolled, 4 remain active including 2 from the 1.0 mg/m² cohort, both having been dose-reduced to 0.8 mg/m².

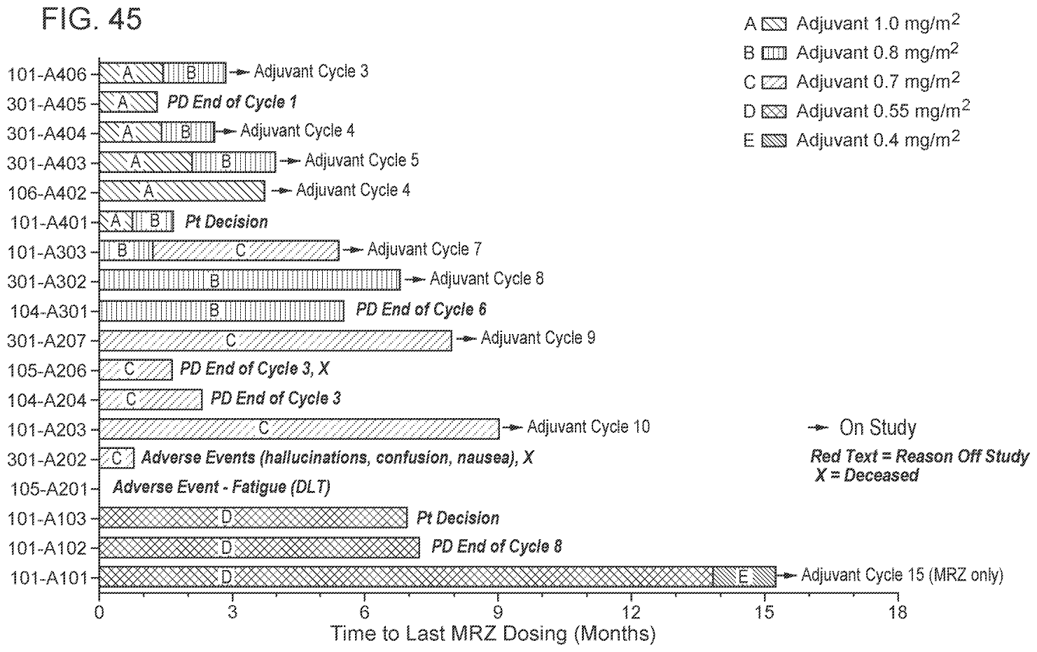

FIG. 45 is a swimmer plot of adjuvant cohort (Stage 1) patients in dose-escalation with dose levels labeled. A DLT (fatigue) in cohort 2 required cohort expansion to N=6 with no other DLTs encountered. Two DLTs were noted in cohort 4 (1 mg/m²). As an enrollment summary; 19 enrolled, 9 remain active including 4 from the 1.0 mg/m² cohort, all have been dose-reduced to 0.8 mg/m².

Thus, 0.8 mg/m² selected as the RP2D in the concomitant and adjuvant cohorts. There was no apparent adverse event “carryover” with DLTs in 5/12 patients at 1.0 mg/m² (i.e. ataxia, confusion, hallucination, delirium) and the dose-expansion portion of protocol is enrolling (N=18) with 0.8 mg/m² MRZ+TMZ+RT followed by 0.8 mg/m² MRZ+TMZ.

TABLE 46
Incidence of Most Frequent Treatment-Related
Adverse Events in Three Lower Dose Cohorts
N = 21 (dose-escalation)

| | Cohort 1 | Cohort 2 | Cohort 3 | Total # Patients |
|---|---|---|---|---|
| | 0.55 | 0.7 | 0.8 | with Grade |
| | mg/m² | mg/m² | mg/m² | 3 Event |
| | (6) | (9) | (6) | (21) |
| Fatigue (26) | 5 | 8 | 5 | 5 |
| Nausea (17) | 3 | 4 | 5 | 0 |
| Vomiting (12) | 0 | 4 | 4 | 1 |
| Constipation (11) | 0 | 6 | 2 | 0 |
| Headache (10) | 2 | 3 | 2 | 0 |
| Anorexia (8) | 2 | 3 | 2 | 0 |
| Diarrhea (7) | 2 | 2 | 2 | 0 |
| Hallucination (11) | 1 | 4 | 2 | 1 |
| Confusion (9) | 1 | 4 | 1 | 0 |
| Ataxia (8) | 1 | 1 | 1 | 1 |
| Dizziness (6) | 2 | 2 | 1 | 0 |

TABLE 47
Incidence of Most Frequent Treatment-Related Adverse Events with Highest Dose Cohort
N = 33 (dose-escalation)

| | | | | Total # Patients | | |
|---|---|---|---|---|---|---|
| | Cohort 1 | Cohort 2 | Cohort 3 | with Grade 3 | Cohort 4 | Total # Patients |
| | 0.55 mg/m² | 0.7 mg/m² | 0.8 mg/m² | Event | 1.0 mg/m² | with Grade 3 |
| | (6) | (9) | (6) | (21) | (12) | Event |
| Fatigue (26) | 5 | 8 | 5 | 5 | 8 | 2 |
| Nausea (17) | 3 | 4 | 5 | 0 | 5 | 0 |
| Vomiting (12) | 0 | 4 | 4 | 1 | 4 | 0 |
| Constipation (11) | 0 | 6 | 2 | 0 | 4 | 0 |
| Headache (10) | 2 | 3 | 2 | 0 | 4 | 2 |
| Anorexia (8) | 2 | 3 | 2 | 0 | 1 | 0 |
| Diarrhea (7) | 2 | 2 | 2 | 0 | 1 | 0 |
| Hallucination (11) | 1 | 4 | 2 | 1 | 4 | 0 |
| Confusion (9) | 1 | 4 | 1 | 0 | 3 | 2 |
| Ataxia (8) | 1 | 1 | 1 | 1 | 4 | 3 |
| Dizziness (6) | 2 | 2 | 1 | 0 | 1 | 0 |

### Example 6

### A Phase 1, Multicenter, Open-Label Study of Marizomib (MRZ) with Temozolomide (TMZ) and Radiotherapy (RT) in Newly Diagnosed WHO Grade IV Malignant Glioma (Glioblastoma, ndGBM)

Proteasome inhibition sensitizes glioma cells to TMZ and RT, providing a novel therapeutic strategy for newly diagnosed glioblastoma (ndGBM).

Purpose: Identify the recommended dose for further studies (RD) for MRZ, an irreversible, brain-penetrant, pan-proteasome inhibitor with anti-glioma preclinical activity, in combination with standard of care (SOC) TMZ and RT in ndGBM.

Methods: Patients enrolled in separate concomitant (TMZ+RT+MRZ at 0.55, 0.7, 0.8, and 1.0 mg/m² dose levels DL) and adjuvant (TMZ+MRZ) cohorts in 3+3 design, followed by dose-expansion at the RD (concomitant followed by adjuvant). MRZ (10 min IV infusion): Concomitant (42 days (D), on D1, 8, 15, 29, 36; Adjuvant (28D-cycle) on D1, 8, 15.

Results: Mean age 58 yrs (73% male) for 33 patients (15 concomitant and 18 adjuvant patients); one DLT (fatigue) at DL 0.7 mg/m² adjuvant, 3 DLTs (ataxia/diarrhea; ataxia/confusion; myocardial infarction) at DL 1.0 mg/m² concomitant and 2 (delirium/ataxia; ataxia/fatigue) at DL 1.0 mg/m² adjuvant. Most common TEAEs (≥20% patients): fatigue, nausea, vomiting, hallucination, ataxia, headache. At least one Grade ≥3 TEAE in 11 of 12 patients at 1.0 mg/m², 1 of 6 at 0.8, 2 of 9 at 0.7 and 2 of 6 at 0.55 mg/m². Severity of TEAEs was generally responsive to dose reductions, supporting a steep dose/response relationship.

Conclusions: A RD of 0.8 mg/m² MRZ in combination with SOC in ndGBM has been chosen. 19 patients are enrolled in the dose-expansion cohort, with 14 patients active; 10 patients from dose-escalation also remain active, with the 5 longest patients currently in adjuvant cycles 12-18 after stopping TMZ. The data demonstrate the combination of MRZ with SOC in ndGBM is well tolerated and without wishing to be bound by theory may bring additional therapeutic benefit to SOC in this unmet need.

### Example 7

### Phase 1b, Multicenter, Open-Label Study of Marizomib Combined with Temozolomide and Radiotherapy in Patients with Newly Diagnosed WHO Grade IV Malignant Glioma (Study of Marizomib with Temozolomide and Radiotherapy in Patients with Newly Diagnosed Brain Cancer)

### General Protocol Information

Study Type: Interventional.

Enrollment: Number of Subjects is 72, anticipated.

Study Design: Study Phase is phase 1. Primary Purpose is treatment. Intervention model is single group. Number of arms is 4. No masking (open-label).

### Summary, Status, Design

Brief Summary: This study is for newly diagnosed WHO Grade IV malignant glioma patients to determine whether an investigational drug known as marizomib (MRZ) will improve the treatment of newly diagnosed glioblastoma patients by delaying the growth of the cancer, reducing the size of the tumor, and/or improving survival. Marizomib (MRZ) is being added to standard-of-care treatments of radiotherapy (RT), temozolomide (TMZ), and Optune.

Detailed Description: Gliomas account for ˜80% of primary malignant tumors in the Central Nervous System (CNS), with WHO Grade IV malignant glioma (G4 MG; including glioblastoma and gliosarcoma) constituting the majority of gliomas, and are essentially incurable. Currently only surgical resection and radiotherapy (RT) with concomitant and adjuvant temozolomide (TMZ) are standard-of-care treatment strategies for newly diagnosed G4 MG. However, resistance to chemotherapy and RT results in a high recurrence rate, with median survival of ˜15-16 months. Since no survival advantage has been demonstrated for the addition of bevacizumab (BEV) to TMZ and RT (Chinot 2014) in newly diagnosed G4 MG, alternative promising investigational agents need to be tested.

Targeting the proteasome is a well-validated target for the treatment of multiple myeloma (MM), and preclinical evidence suggests that targeting the proteasome in glioma cells shows significant anti-tumor activity. Proteasome activity is elevated in patient-derived glioblastoma (GBM) tissue in comparison with normal human brain. Importantly, preclinical evidence demonstrates that proteasome inhibition sensitizes GBM cell lines to irradiation and to TMZ. Further, the combination of bortezomib (BTZ, one of three proteasome inhibitors [PI] currently approved for the treatment of MM) with TMZ resulted in synergistic glioblastoma cell death in vitro, and BTZ reduces glioma cell survival in vitro in cell lines sensitive and resistant to TMZ.

Despite the activity against GBM cells in vitro, BTZ does not cross the blood brain barrier, and thus has proven ineffective in animal models and in the clinic. In contrast, marizomib (MRZ)—a potent and irreversible 20S PI possesses the unique attribute among PIs to cross the blood brain barrier as shown in previous clinical studies. These data prompted examination of the combination of MRZ and BEV in an ongoing clinical trial in patients with recurrent G4 MG. In the dose-escalation portion of this ongoing study (MRZ-108), 12 patients were dosed with MRZ once weekly for 3 weeks (0.55, 0.7, and 0.8 mg/m² infused intravenously (IV) over 10 minutes) and with BEV on weeks 1 and 3 (10 mg/kg IV) of a 28-day cycle. As of April 2016, of these 12 patients, 7 were on study for over 4 months-5 with a partial response (including 2 patients with no radiologic evidence of tumor on 2 or more consecutive MRI scans) and 2 patients whose best response was stable disease. Four of these 12 patients were treated for over 6 months, 3 of whom remain on study. The recommended Phase 2 dose (RP2D) of MRZ was determined to be 0.8 mg/m². Currently, an expansion cohort of 24 patients has been enrolled in the Phase 2 portion of the study. The next phase involves treatment with MRZ alone (no BEV) in patients with recurrent G4 MG, and has begun enrolling patients in the second quarter of 2016.

Together, the demonstrated activity of PIs in preclinical glioma models, and the synergistic activity of PIs with TMZ on glioblastoma cells, along with the ability of MRZ to access the CNS, provides compelling rationale to assess the therapeutic benefit of the combination of MRZ with TMZ in patients with G4 MG, for whom no brain-penetrant options for proteasome inhibition are currently available.

Very recently, the FDA has approved a novel treatment device using tumor treating fields (Optune) in addition to standard of care RTand TMZ as an option to standard of care. Optune has been shown to significantly improve both progression-free and overall survival in GBM patients. An additional cohort of 12 patients will be treated with Optune in combination with MRZ and TMZ.

Overall Status: Recruiting

Study Start Date: Aug. 31, 2016 (Actual).

Primary Completion Date: Jun. 18, 2018 (Anticipated).

Study Completion Date: Dec. 31, 2019 (Anticipated).

Outcome Measures: Primary Outcome Measures: (1) Determine MRZ maximum tolerated dose (MTD) and recommended phase 2 dose (RP2D) for both concomitant treatment (MRZ+TMZ+RT) and adjuvant treatment (MRZ+TMZ). Time Frame: 42-day concomitant treatment and 28-day Cycle 1 adjuvant treatment. Description: Assess dose-limiting toxicities (DLTs) in each dose-escalation arm.

(2) To assess adverse events during the adjuvant treatment. Time Frame: From the first dose of study drug through 28 days after the last dose. Description: To assess the safety of the combination of MRZ and TMZ with the addition of OptuneTM in patients entering Adjuvant Treatment

Secondary Outcome Measures: (1) To confirm the MRZ RP2D for concomitant and adjuvant treatment in an expanded group of patients. Time Frame: Assessments made during the concomitant (dosing for 42 days of a 10-week treatment period) and adjuvant (one or more 28-day cycles) treatment periods in the dose-expansion stage of the study. Description: Assess adverse events.

(2) Assess adverse events during concomitant and adjuvant treatment. Time Frame: From the first dose of study drug through 28 days after the last dose. Description: Assess adverse events.

(3) Evaluate the activity (overall survival [OS]) of MRZ+TMZ+RT. Time Frame: Survival monitored throughout the concomitant and adjuvant treatment periods and every three months during long-term follow-up for 2 years. Description: Includes death due to any cause.

(4) Evaluate the activity (progression-free survival [PFS]) of MRZ+TMZ+RT. Time Frame: MRI assessments at Week 10 during concomitant treatment and every even cycle during adjuvant treatment, death monitored throughout the treatment periods, and disease progression and death monitored every three months during long-term follow-up for 2 years. Description: RANO criteria used to assess tumor response.

(5) MRZ pharmacokinetics—Maximum Serum Concentration (Cₘₐₓ). Time Frame: Day 1 and Day 8 during Stage 1 (dose-escalation). Description: Measured after stopping the MRZ infusion.

(7) MRZ pharmacokinetics—Elimination Half-Life (t_{1/2}). Time Frame: Day 1 and Day 8 during Stage 1 (dose-escalation). Description: Calculated from MRZ serum concentrations measured through 60 minutes after the stopping the infusion.

(8) MRZ pharmacokinetics—Area Under the Blood Concentration-Time Curve (AUC₀₋ₜ, AUC_{0-inf}). Time Frame: Day 1 and Day 8 during Stage 1 (dose-escalation). Description: Calculated from MRZ serum concentrations measured through 60 minutes after the stopping the infusion.

(9) MRZ pharmacokinetics—Clearance (CL). Time Frame: Day 1 and Day 8 during Stage 1 (dose-escalation). Description: Calculated from MRZ serum concentrations measured through 60 minutes after the stopping the infusion.

(10) MRZ pharmacokinetics—Volume of Distribution (Vd). Time Frame: Day 1 and Day 8 during Stage 1 (dose-escalation). Description: Calculated from MRZ serum concentrations measured through 60 minutes after the stopping the infusion.

(11) TMZ serum concentration. Time Frame: On Day 1 of Week 1 (D1) and on Day 1 of Week 2 (D8), TMZ serum concentration will be measured before treatment, and 60 minutes after the dose and 24 hrs after the dose (prior to the Day 9 TMZ dose). Description: Peak and trough TMZ serum concentrations will be measured to see if MRZ affects TMZ serum concentration.

(12) Assess neurological coordination using the Scale for the Assessment and Rating for Ataxia (SARA). Time Frame: Assessments made at baseline and then weeks 1, 5, and 8 during concomitant treatment, on Day 1 of each Cycle during adjuvant treatment, and at the end of treatment visit (28 days after last dose of study drug). Description: Investigator evaluation of neurologic coordination using a standardized rating scale.

(13) Evaluate the activity (overall survival [OS]) of MRZ+TMZ+Optune. Time Frame: Survival monitored throughout the concomitant and adjuvant treatment periods and every three months during long-term follow-up for 2 years. Description: Includes death due to any cause.

(14) Evaluate the activity (progression-free survival [PFS]) of MRZ+TMZ+Optune. Time Frame: MRI assessments at Week 10 during concomitant treatment and every even Cycle during adjuvant treatment, death monitored throughout the treatment periods, and disease progression and death monitored every three months during long-term follow-up for 2 years. Description: RANO 2010 criteria used to assess tumor response.

Conditions: Glioblastoma, malignant glioma.

Keywords: newly diagnosed; malignant glioma; WHO Grade 4; WHO Grade IV; Marizomib; MRZ; TMZ; RT; brain cancer; proteasome inhibitor; radiation; temozolomide; temodar; chemotherapy; concurrent; adjuvant; Optune; Novocure; NovoTTF.

Arms/Interventions: Stage 1: Concomitant Treatment. Arm Type: Experimental. Arm Description: MRZ+TMZ+RT Patients who complete Concomitant Treatment may continue on to Adjuvant Treatment.

Stage 1: Adjuvant Treatment. Arm type: Experimental. Arm Description: MRZ+TMZ. Intervention Type: Drug. Intervention Name: MRZ. Intervention Description: MRZ dose ranges from 0.55 to 1.2 mg/m² given IV over 10 minutes on Days 1, 8, 15, 29, and 36 during Concomitant Treatment. MRZ dose ranges from 0.55 to 1.2 mg/m² given IV over 10 minutes on Days 1, 8, 15 every 28 days during Adjuvant Treatment. IV hydration will be given prior to the MRZ infusion.

Intervention Type: Drug. Intervention Name: TMZ. Intervention Description: TMZ will be administered once daily, 7 days/week, for 6 weeks, starting on Day 1, at a dose of 75 mg/m² during Concomitant Treatment. TMZ will be administered once daily on Days 1-5 every cycle, dose range 150 to 200 mg/m² during Adjuvant Treatment. Other Names: temozolomide Temodar

Stage 2: Dose-Expansion. Arm Type: Experimental. Arm Description: MRZ+TMZ+RT followed by MRZ+TMZ. In Stage 2 (dose-expansion): a minimum of 12 and up to approximately 18 additional evaluable patients will be enrolled in a cohort in which Concomitant Treatment (MRZ+TMZ+RT) is followed by Adjuvant Treatment (MRZ+TMZ) to confirm the MTD for each treatment regimen as determined in the Dose-Escalation (Stage 1), and to assess preliminary activity of the recommended Phase 2 dose (RP2D).

Intervention type: Drug. Intervention Name: MRZ. Intervention Description: MRZ dose ranges from 0.55 to 1.2 mg/m² given IV over 10 minutes on Days 1, 8, 15, 29, and 36 during Concomitant Treatment. MRZ dose ranges from 0.55 to 1.2 mg/m² given IV over 10 minutes on Days 1, 8, 15 every 28 days during Adjuvant Treatment. IV hydration will be given prior to the MRZ infusion.

Intervention Type: Drug. Intervention Name: TMZ. Intervention Description: TMZ will be administered once daily, 7 days/week, for 6 weeks, starting on Day 1, at a dose of 75 mg/m² during Concomitant Treatment. TMZ will be administered once daily on Days 1-5 every cycle, dose range 150 to 200 mg/m² during Adjuvant Treatment. Other Names: temozolomide Temodar

Intervention Type: Radiation. Intervention Name: RT. Intervention Description: Focal RT will be administered once daily, 5 days/week, for 30 doses over 6 weeks to a total dose of 60 Gy, starting on Day 1 during Concomitant Treatment. Other Names: radiation therapy.

Optune Arm. Arm Type: Experiment. Arm Description: MRZ+TMZ+Optune. Intervention Type: Device. Intervention Name: Optune. Intervention Description: Tumor Treating Fields Therapy device to be worn ≥18 hours per day. Other Names: NovoTTF-100A.

### Eligibility

Eligibility Criteria Inclusion Criteria: Signed Informed Consent Form. Males and females of age ≥18 years or of age ≥22 years for those assigned to Optune™ at the time of signing of the informed consent document. Histologically confirmed newly diagnosed G4 MG. Karnofsky Performance Status (KPS) score ≥70%. For Concomitant Treatment: Prior tumor resection or biopsy up to 8 weeks prior to first MRZ dose. For Adjuvant Treatment: All AEs resulting from surgery must have resolved to NCI-CTCAE (v. 4.03) Grade ≤1. Stable or decreasing dose of corticosteroids over 14 days prior to first MRZ dose. For Concomitant Treatment: No prior treatment with MRZ or any other PIs, including BTZ, carfilzomib (CFZ), or ixazomib (IXZ). For Adjuvant Treatment: No prior treatment with BTZ, CFZ, or IXZ. No investigational agent within 4 weeks prior to first dose of study drug. Adequate hematological, renal, and hepatic function. Patients must be without seizures for at least 14 days prior to enrollment, and patients who receive treatment with AEDs must be on stable doses for at least 14 days prior to enrollment. Absence of known HIV infection, chronic hepatitis B, or hepatitis C infection; absence of any other serious medical condition which could interfere with oral medication intake. Patients with archival tumor tissue suitable for measurement of proteasome activity and biomarker status must give permission to access and test the tissue. Patients without archival tumor tissue are eligible for the Dose-Escalation stage, but not the Dose-Expansion stage of the study. For women of child-bearing potential and for men with partners of child-bearing potential, patient must agree to take contraceptive measures for duration of treatments and for one month after last study treatment. Willing and able to adhere to the study visit schedule and other protocol requirements.

Exclusion Criteria: Co-medication or concomitant therapy that may interfere with study results. History of thrombotic or hemorrhagic stroke or myocardial infarction within 6 months. Other chemotherapy or anti-tumor treatment for brain tumor (other than therapies required by the inclusion criteria of this protocol). Pregnant or breast feeding. Uncontrolled intercurrent illness that would limit compliance with study requirements, or disorders associated with significant immunocompromised state. Known other previous/current malignancy requiring treatment within ≤3 years except for limited disease treated with curative intent. Any comorbid condition that confounds the ability to interpret data from the study as judged by the Investigator or Medial Monitor. For those enrolled in Adjuvant Treatment with Optune™, patients are excluded if they are <22 years of age, have an active implanted medical device, a skull defect, bullet fragments in the head, sensitivity to conductive hydrogels, a scalp condition that might interfere with wearing the device, or GBM that is not supratentorial.

Gender: Both

Gender Based: No

Minimum Age: 18 years

Maximum Age: No limit

Accept Healthy Volunteers? No

### Example 8

### A Phase 1, Multicenter, Open-Label Study of Marizomib (MRZ) with Temozolomide (TMZ) and Radiotherapy (RT) in Newly-Diagnosed WHO Grade IV Malignant Glioma (Glioblastoma, ndGBM): Full Enrollment Results

Background: Proteasome inhibition sensitizes glioma cells to TMZ and RT, providing a novel therapeutic strategy for ndGBM. The purpose of the trial is to identify the recommended dose for further studies (RD) for MRZ—an irreversible, brain-penetrant, pan-proteasome inhibitor with anti-glioma preclinical activity—combined with standard-of-care (SOC) TMZ and RT in ndGBM.

Methods: Patients enrolled in separate concomitant (TMZ/RT+MRZ) and adjuvant (TMZ+MRZ) treatment cohorts in dose-escalation (3+3 design, MRZ at 0.55, 0.7, 0.8, and 1.0 mg/m²), followed by dose-expansion at the RD in concomitant followed by adjuvant treatment. MRZ (10 min IV infusion): Concomitant Day 1, 8, 15, 29, 36; Adjuvant (28D-cycle) Day 1, 8, 15.

Results as of Feb. 1, 2018: Fully-enrolled dose-escalation cohorts (15 concomitant and 18 adjuvant patients) mean age 58 yrs (73% male); one DLT (fatigue) in 0.7 mg/m² adjuvant cohort, 3 (ataxia/diarrhea; ataxia/confusion; myocardial infarction) in 1.0 mg/m² concomitant and 2 (delirium/ataxia; ataxia/fatigue) in 1.0 mg/m² adjuvant cohorts. Most common TEAEs (≥20% patients): fatigue, nausea, vomiting, hallucination, ataxia, headache. At least one Grade ≥3 TEAE in 11 of 12 patients at 1.0 mg/m² including one Grade 4 and one Grade 5 TEAE; at 0.8, 0.7, and 0.55 mg/m² MRZ, Grade 3 TEAEs in 2 of 6, 4 of 9, and 3 of 6 patients.

Conclusions: MRZ demonstrated a steep safety dose-response; TEAEs/DLTs were most commonly CNS adverse events (ataxia, hallucinations) and generally dose-related, short-lasting, reversible and ameliorated by subsequent dose reductions. The RD of 0.8 mg/m² MRZ in combination with SOC in ndGBM was chosen based on dose-escalation findings. An additional arm at the MRZ RD with adjuvant TMZ+Optune is also enrolling to assess the safety and tolerability of this combination. Twenty (20) patients are currently enrolled in dose-expansion arm at RD, with 15 patients active; in addition, 9 patients from dose-escalation also remain active, with the 5 longest patients on study 12-18 months. Based on the safety and tolerability of MRZ in this study, an EORTC-sponsored phase 3 study will open in April 2018 to assess the overall survival benefit of MRZ with TMZ/RT→4 TMZ in ndGBM.

### Equivalents

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

### Enumerated Embodiments

1. A method of treating a central nervous system cancer in a subject in need thereof, the method comprising a treatment regimen comprising administering to the subject a therapeutic amount of a proteasome inhibitor, wherein the therapeutic amount, in the context of the treatment regimen, is sufficient for the subject to experience at least one central nervous system-related adverse event and wherein administration of the therapeutic amount is continued once the adverse event is triggered.
2. The method of claim 1, wherein the central nervous system-related adverse event is triggered in the cerebellum, brain, or brain stem.
3. The method of claim 1, wherein the proteasome inhibitor is capable of crossing the blood-brain barrier.
4. The method of claim 1, wherein the proteasome inhibitor is marizomib.
5. The method of claim 1, wherein the central nervous system cancer is glioma.
6. The method of claim 5, wherein the glioma is recurrent glioma.
7. The method of claim 5, wherein the glioma is grade IV malignant glioma.
8. The method of claim 5, wherein the glioma is glioblastoma.
9. The method of claim 1, wherein the subject experiences at least one central nervous system-related adverse event selected from the group consisting of ataxia, gait disturbance, fall, dysarthria, dizziness, and hallucination.
10. The method of claim 1, wherein the subject experiences at least two central nervous system-related adverse events selected from the group consisting of ataxia, gait disturbance, fall, dysarthria, dizziness, and hallucination.
11. The method of claim 1, wherein the subject experiences at least three central nervous system-related adverse events selected from the group consisting of ataxia, gait disturbance, fall, dysarthria, dizziness, and hallucination.
12. The method of claim 1, wherein the subject experiences at least four central nervous system-related adverse events selected from the group consisting of ataxia, gait disturbance, fall, dysarthria, dizziness, and hallucination.
13. The method of claim 1, wherein the subject experiences at least five central nervous system-related adverse events selected from the group consisting of ataxia, gait disturbance, fall, dysarthria, dizziness, and hallucination.
14. The method of claim 1, wherein the subject experiences all of ataxia, gait disturbance, fall, dysarthria, dizziness, and hallucination.
15. The method of claim 9, wherein the dizziness is balance disorder.
16. The method of claim 9, wherein the subject further experiences a central nervous system-related adverse event selected from the group consisting of agitation, anxiety, aphasia, apraxia, cognitive disorder, concentration impairment, confusional state, convulsion, delirium, delusion, depressed level of consciousness, depression, facial nerve disorder, facial paresis, fatigue, insomnia, intention tremor, irritability, memory impairment, mental status change, personality change, psychotic disorder, pyramidal tract syndrome, somnolence, suicidal ideation, tremor, trigeminal nerve disorder, vertigo, or a combination thereof.
17. The method of claim 1, wherein the subject is administered increasing amounts of the proteasome inhibitor until the subject experiences the central nervous system-related adverse event.
18. The method of claim 1, wherein administration of the proteasome inhibitor to the subject is continued after the subject experiences the central nervous system-related adverse event.
19. The method of claim 17, wherein the dose of the proteasome inhibitor is not lowered after the subject experiences the central nervous system-related adverse event.
20. The method of claim 1, wherein the adverse event is at least a grade 1 adverse event.
21. The method of claim 1, wherein the adverse event is at least a grade 2 adverse event.
22. The method of claim 1, wherein the adverse event is at least a grade 3 adverse event.
23. The method of claim 1, wherein the adverse event is at least a grade 4 adverse event.
24. The method of claim 1, wherein the proteasome inhibitor is administered weekly.
25. The method of claim 1, wherein the proteasome inhibitor is administered in combination with bevacizumab.
26. A method of determining therapeutic amount of a proteasome inhibitor for the treatment of a central nervous system cancer in a subject in need thereof, the method comprising a treatment regimen comprising administering to the subject the proteasome inhibitor at increasing dose amounts until the subject experiences at least one central nervous system-related adverse event, wherein the therapeutic amount, in the context of the treatment regimen, is the amount at which the subject experiences the central nervous system-related adverse event.
27. The method of claim 26, wherein the central nervous system-related adverse event is triggered in the cerebellum, brain, or brain stem.
28. The method of claim 26, wherein the subject is administered a first subsequent dose of proteasome inhibitor if no adverse events are experienced by the subject after being administered an initial dose of proteasome inhibitor, wherein the first subsequent dose comprises a greater amount of proteasome inhibitor than the initial dose.
29. The method of claim 27, wherein the subject is administered a second subsequent dose of proteasome inhibitor if no central nervous system-related adverse events are experienced by the subject after being administered the first subsequent dose, wherein the second subsequent dose comprises a greater amount of proteasome inhibitor than the first subsequent dose.
30. The method of claim 26, wherein the initial dose is about 0.55 mg/m² of proteasome inhibitor.
31. The method of claim 26, wherein the first subsequent dose is about 0.7 mg/m² of proteasome inhibitor.
32. The method of claim 26, wherein the second subsequent dose is about 0.8 mg/m² of proteasome inhibitor.
33. The method of claim 26, wherein the initial dose is about 0.8 mg/m² of proteasome inhibitor.
34. The method of claim 26, wherein the first subsequent dose is about 1.1 mg/m² of proteasome inhibitor.
35. The method of claim 26, wherein the first subsequent dose is about 1.2 mg/m² of proteasome inhibitor.
36. The method of claim 26, wherein the proteasome inhibitor is capable of crossing the blood-brain barrier.
37. The method of claim 26, wherein the proteasome inhibitor is marizomib.
38. The method of claim 26, wherein the central nervous system cancer is glioma.
39. The method of claim 38, wherein the glioma is recurrent glioma.
40. The method of claim 39, wherein the glioma is grade IV malignant glioma.
41. The method of claim 40, wherein the glioma is glioblastoma.
42. The method of claim 26, wherein the subject experiences at least one central nervous system-related adverse event selected from the group consisting of ataxia, gait disturbance, fall, dysarthria, and dizziness, and hallucination or a combination thereof.
43. The method of claim 26, wherein the subject experiences at least two central nervous system-related adverse events selected from the group consisting of ataxia, gait disturbance, fall, dysarthria, and dizziness, and hallucination or a combination thereof.
44. The method of claim 26, wherein the subject experiences at least three central nervous system-related adverse events selected from the group consisting of ataxia, gait disturbance, fall, dysarthria, and dizziness, and hallucination or a combination thereof.
45. The method of claim 26, wherein the subject experiences at least four central nervous system-related adverse events selected from the group consisting of ataxia, gait disturbance, fall, dysarthria, and dizziness, and hallucination or a combination thereof.
46. The method of claim 26, wherein the subject experiences at least five central nervous system-related adverse events selected from the group consisting of ataxia, gait disturbance, fall, dysarthria, and dizziness, and hallucination or a combination thereof.
47. The method of claim 26, wherein the subject experiences ataxia, gait disturbance, fall, dysarthria, and dizziness, and hallucination.
48. The method of claim 42, wherein the at least one adverse event furthers includes hallucination, agitation, anxiety, aphasia, apraxia, cognitive disorder, concentration impairment, confusional state, convulsion, delirium, delusion, depressed level of consciousness, depression, facial nerve disorder, facial paresis, fatigue, insomnia, intention tremor, irritability, memory impairment, mental status change, personality change, psychotic disorder, pyramidal tract syndrome, somnolence, suicidal ideation, tremor, trigeminal nerve disorder, vertigo, or a combination thereof.
49. The method of claim 26, wherein the adverse event is at least a grade 1 adverse event.
50. The method of claim 26, wherein the adverse event is at least a grade 2 adverse event.
51. The method of claim 26, wherein the adverse event is at least a grade 3 adverse event.
52. The method of claim 26, wherein the adverse event is at least a grade 4 adverse event.
53. The method of claim 26, wherein the proteasome inhibitor is administered weekly.
54. The method of claim 1, wherein the therapeutic amount is sufficient to treat a cancer in subjects with a methylated MGMT promoter.
55. The method of claim 1, wherein the therapeutic amount is sufficient to treat a cancer in subjects with an un-methylated MGMT promoter.
56. The method of claim 26, wherein the therapeutic amount is sufficient to treat a cancer in subjects with a methylated MGMT promoter.
57. The method of claim 26, wherein the therapeutic amount is sufficient to treat a cancer in subjects with an un-methylated MGMT promoter.
58. The method of claim 1, wherein the treatment regimen is the proteasome inhibitor alone.
59. The method of claim 1, wherein the treatment regimen comprises the proteasome inhibitor in combination with an additional therapeutic agent.
60. The method of claim 59, wherein the additional therapeutic agent is bevacizumab.
61. The method of claim 58 or 59, wherein the proteasome inhibitor is marizomib alone.
62. The method of claim 26, wherein the treatment regimen is the proteasome inhibitor alone.
63. The method of claim 26, wherein the treatment regimen comprises the proteasome inhibitor in combination with an additional therapeutic agent.
64. The method of claim 63, wherein the additional therapeutic agent is bevacizumab.
65. The method of claim 62 or 63, wherein the proteasome inhibitor is marizomib.

1. A method of treating a central nervous system cancer in a subject in need thereof, the method comprising a treatment regimen comprising administering to the subject a therapeutic amount of a proteasome inhibitor, wherein the therapeutic amount, in the context of the treatment regimen, is sufficient for the subject to experience at least one central nervous system-related adverse event and wherein administration of the therapeutic amount is continued once the adverse event is triggered.

2. The method of claim 1, wherein the proteasome inhibitor is marizomib.

3. The method of claim 1, wherein the glioma is grade IV malignant glioma.

4. The method of claim 1, wherein the proteasome inhibitor is administered once per week.

5. The method of claim 1, wherein the treatment regimen comprises the proteasome inhibitor in combination with an additional therapeutic agent.

6. The method of claim 5, wherein the additional therapeutic agent is bevacizumab.

7. The method of claim 1, wherein the proteasome inhibitor is administered at an initial dosage of about 0.8 mg/m².

8. The method of claim 7, wherein the proteasome inhibitor is administered at subsequent dosages of about 0.8 mg/m².

9. The method of claim 1, wherein the proteasome inhibitor is administered on days 1, 8, and 15 of a 28-day cycle.

10. The method of claim 9 further comprising administering bevacizumab on days 1 and 14 of a 28-day cycle.

11. The method of claim 10 wherein the bevacizumab is administered at a dose of about 10 mg/kg.

12. The method of claim 5, wherein the additional therapeutic agent is temozolomide.

13. The method of claim 12, wherein the therapeutic regimen further comprises administration of radiotherapy.

14. The method of claim 13, wherein the therapeutic regimen comprises administration of OPTUNE.

15. The method of claim 1, wherein the proteasome inhibitor is administered on days 1, 8, 15, 29 and 36.

16. The method of claim 12, wherein the temozolomide is administered every day at a dosage of 75 mg/m².

17. The method of claim 16, wherein the temozolomide is administered for six weeks.

18. The method of claim 12, wherein the temozolomide is administered on five consecutive days a week for a 28-day cycle at a dosage of between about 150 mg/m² to about 200 mg/m².

19. The method of claim 18, wherein the temozolomide is administered for about 12 cycles.

20. The method of claim 13, wherein the radiotherapy is administered once daily for five days a week at a dose of about 60 Gy.

21. The method of claim 20, wherein the radiotherapy is administered for about 6 weeks.
